(19) United States
(12) Patent Application Publication
(10) Pub. No.: US 2021/0040038 A1
(43) Pub. Date: Feb. 11, 2021

(54) NOVEL HETEROCYCLIC COMPOUND AND ORGANIC LIGHT EMITTING DEVICE COMPRISING THE SAME

(71) Applicant: LG CHEM, LTD., Seoul (KR)

(72) Inventors: Sang Duk SUH, Daejeon (KR); Wanpyo HONG, Daejeon (KR); Sung Kil HONG, Daejeon (KR)

(21) Appl. No.: 16/609,186

(22) PCT Filed: May 31, 2018
(86) PCT No.: PCT/KR2018/006242

§ 371 Date: Oct. 28, 2019

(30) Foreign Application Priority Data
Jun. 29, 2017 (KR) 10-2017-0082774
May 30, 2018 (KR) 10-2018-0062156

(51) Int. Cl. C07D 209/82; C07D 403/14; C07D 409/14; C07D 405/14; H01L 51/00

(52) U.S. Cl. CPC C07D 209/82; C07D 403/14; C07D 409/14; H01L 51/0061; H01L 51/0067; H01L 51/0072; C07D 405/14

(57) ABSTRACT

Provided is a heterocyclic compound of Chemical Formula 1:

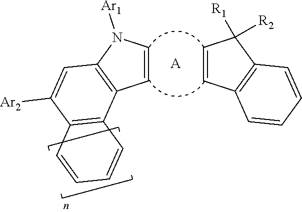

and an organic light emitting device including the same.

### CROSS-REFERENCE TO RELATED APPLICATION(S)

This application claims the benefits of the filing dates of Korean Patent Application No. 10-2017-0082774 filed with the Korean Intellectual Property Office on Jun. 29, 2017, and Korean Patent Application No. 10-2018-0062156 filed with the Korean Intellectual Property Office on May 30, 2018, the entire contents of which are incorporated herein by reference.

### TECHNICAL FIELD

The present invention relates to a novel heterocyclic compound and to an organic light emitting device including the same.

### BACKGROUND ART

In general, an organic light emitting phenomenon is one in which electrical energy is converted into light energy by using an organic material. The organic light emitting device using the organic light emitting phenomenon has characteristics such as a wide viewing angle, excellent contrast, a fast response time, and excellent luminance, driving voltage, and response speed, and thus many studies have proceeded thereon.

The organic light emitting device generally has a structure which includes an anode, a cathode, and an organic material layer interposed between the anode and the cathode. The organic material layer frequently has a multilayered structure that includes different materials in order to enhance efficiency and stability of the organic light emitting device, and for example, the organic material layer can be formed of a hole injection layer, a hole transport layer, a light emitting layer, an electron transport layer, an electron injection layer, and the like. In the structure of the organic light emitting device, if a voltage is applied between two electrodes, the holes are injected from an anode into the organic material layer and the electrons are injected from the cathode into the organic material layer, and when the injected holes and electrons meet each other, an exciton is formed, and light is emitted when the exciton falls from an excited state to a ground state.

There is a continuing need for the development of new materials for the organic materials used in these organic light emitting devices.

### BACKGROUND ART LITERATURE

### [Patent Literature]

(Patent Literature 0001) Korean Patent Laid-open Publication No. 10-2000-0051826

### DETAILED DESCRIPTION OF THE INVENTION

### Technical Problem

It is an object of the present invention to provide a novel heterocyclic compound and an organic light emitting device including the same.

### Technical Solution

In one aspect of the invention, a compound represented by the following Chemical Formula 1 is provided.

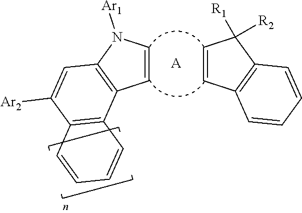

In Chemical Formula 1,

A is a benzene ring fused to two adjacent rings,

R₁ and R₂ are each independently a substituted or unsubstituted C₁₋₆₀ alkyl, or a substituted or unsubstituted C₆₋₆₀ aryl, and

Ar₁ is phenyl, biphenyl, naphthyl, or any one of the following Chemical Formulae 2-1 to 2-5:

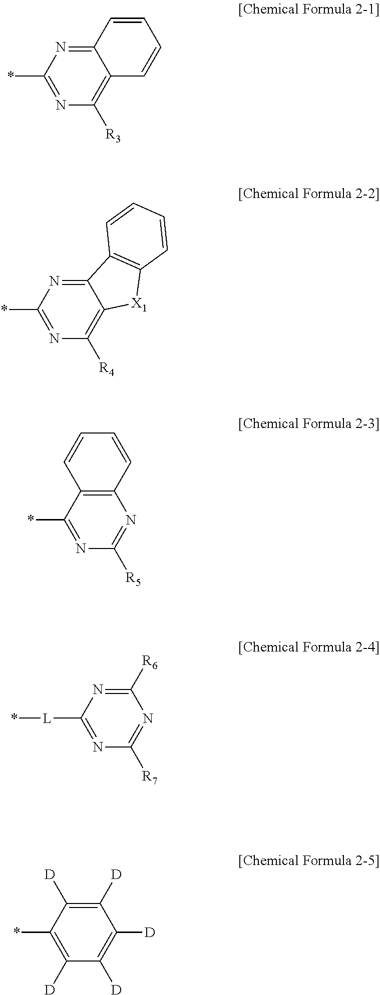

wherein, in Chemical Formulae 2-1, 2-2, 2-3, 2-4, and 2-5,

R₃, R₄, R₅, R₆, and R₇ are each independently phenyl, biphenyl, or naphthyl,

X₁ is O or S,

L is a single bond or phenylene,

Ar₂ is

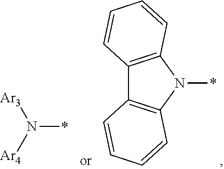

Ar₃ and Ar₄ are each independently a substituted or unsubstituted C₆₋₆₀ aryl, and

n is 0 or 1.

In another aspect of the invention, an organic light emitting device is provided, including: a first electrode; a second electrode provided opposite to the first electrode; and one or more organic material layers provided between the first electrode and the second electrode, wherein one or more layers of the organic material layers includes the compound represented by Chemical Formula 1.

### Advantageous Effects

The compound represented by Chemical Formula 1 described above can be used as a material of an organic material layer of an organic light emitting device, and can improve efficiency, achieve a low driving voltage, and/or improve lifetime characteristics in the organic light emitting device.

In particular, the compound represented by Chemical Formula 1 described above can be used as a material for hole injection, hole transport, hole injection and transport, light emitting, electron transport, or electron injection.

### BRIEF DESCRIPTION OF DRAWINGS

FIG. 1 shows an example of an organic light emitting device including a substrate 1, an anode 2, a light emitting layer 3, and a cathode 4.

FIG. 2 shows an example of an organic light emitting device including a substrate 1, an anode 2, a hole injection layer 5, a hole transport layer 6, a light emitting layer 7, an electron transport layer 8, and a cathode 4.

### DETAILED DESCRIPTION OF THE EMBODIMENTS

Hereinafter, the present invention will be described in more detail to help understanding of the present invention.

As used herein, the notations *⁻ and

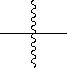

mean a bond linked to another substituent group.

As used herein, the term “substituted or unsubstituted” means being unsubstituted or substituted with one or more substituents selected from the group consisting of: deuterium; a halogen group; a nitrile group; a nitro group; a hydroxy group; a carbonyl group; an ester group; an imide group; an amino group; a phosphine oxide group; an alkoxy group; an aryloxy group; an alkylthioxy group; an arylthioxy group; an alkylsulfoxy group; an arylsulfoxy group; a silyl group; a boron group; an alkyl group; a cycloalkyl group; an alkenyl group; an aryl group; an aralkyl group; an aralkenyl group; an alkylaryl group; an alkylamine group; an aralkylamine group; a heteroarylamine group; an arylamine group; an arylphosphine group; and a hetero-cyclic group containing at least one of N, O, and S atoms, or being unsubstituted or substituted with a substituent to which two or more substituents are linked among the substituents exemplified above. For example, “the substituent to which two or more substituents are linked” can be a biphenyl group. That is, the biphenyl group can also be an aryl group, and can be interpreted as a substituent to which two phenyl groups are linked.

In the present specification, the number of carbon atoms of a carbonyl group is not particularly limited, but is preferably 1 to 40. Specifically, the carbonyl group can be a compound having the following structural formulae, but is not limited thereto.

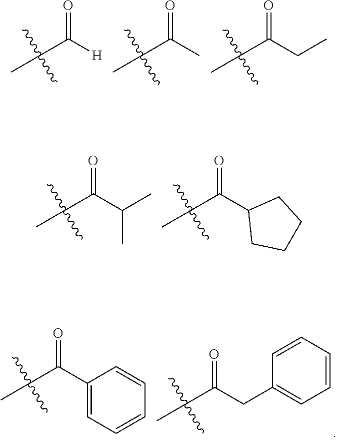

In the present specification, for an ester group, the oxygen of the ester group can be substituted with a straight-chain, branched-chain, or cyclic alkyl group having 1 to 25 carbon atoms, or an aryl group having 6 to 25 carbon atoms. Specifically, the ester group can be a compound having the following structural formulae, but is not limited thereto.

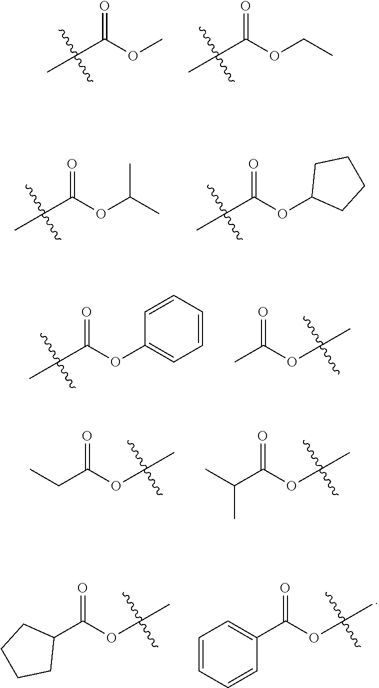

In the present specification, the number of carbon atoms of an imide group is not particularly limited, but is preferably 1 to 25. Specifically, the imide group can be a compound having the following structural formulae, but is not limited thereto.

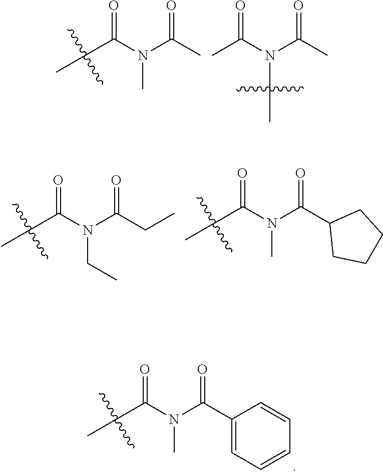

In the present specification, a silyl group specifically includes a trimethylsilyl group, a triethylsilyl group, a t-butyldimethylsilyl group, a vinyldimethylsilyl group, a propyldimethylsilyl group, a triphenylsilyl group, a diphenylsilyl group, a phenylsilyl group, and the like, but is not limited thereto.

In the present specification, a boron group specifically includes a trimethylboron group, a triethylboron group, a t-butyldimethylboron group, a triphenylboron group, and a phenylboron group, but is not limited thereto.

In the present specification, examples of a halogen group include fluorine, chlorine, bromine, and iodine.

In the present specification, the alkyl group can be a straight chain or branched chain, and the number of carbon atoms thereof is not particularly limited, but is preferably 1 to 40. According to one embodiment, the number of carbon atoms of the alkyl group is 1 to 20. According to another embodiment, the number of carbon atoms of the alkyl group is 1 to 10. According to another embodiment, the number of carbon atoms of the alkyl group is 1 to 6. Specific examples of the alkyl group include methyl, ethyl, propyl, n-propyl, isopropyl, butyl, n-butyl, isobutyl, tert-butyl, sec-butyl, 1-methyl-butyl, 1-ethyl-butyl, pentyl, n-pentyl, isopentyl, neopentyl, tert-pentyl, hexyl, n-hexyl, 1-methylpentyl, 2-methylpentyl, 4-methyl-2-pentyl, 3,3-dimethylbutyl, 2-ethylbutyl, heptyl, n-heptyl, 1-methylhexyl, cyclopentylmethyl, cycloheptylmethyl, octyl, n-octyl, tert-octyl, 1-methylheptyl, 2-ethylhexyl, 2-propylpentyl, n-nonyl, 2,2-dimethylheptyl, 1-ethyl-propyl, 1,1-dimethyl-propyl, isohexyl, 2-methylpentyl, 4-methylhexyl, 5-methylhexyl, and the like, but are not limited thereto.

In the present specification, the alkenyl group can be a straight chain or branched chain, and the number of carbon atoms thereof is not particularly limited, but is preferably 2 to 40. According to one embodiment, the number of carbon atoms of the alkenyl group is 2 to 20. According to another embodiment, the number of carbon atoms of the alkenyl group is 2 to 10. According to still another embodiment, the number of carbon atoms of the alkenyl group is 2 to 6. Specific examples thereof include vinyl, 1-propenyl, isopropenyl, 1-butenyl, 2-butenyl, 3-butenyl, 1-pentenyl, 2-pentenyl, 3-pentenyl, 3-methyl-1-butenyl, 1,3-butadienyl, allyl, 1-phenylvinyl-1-yl, 2-phenylvinyl-1-yl, 2,2-diphenylvinyl-1-yl, 2-phenyl-2-(naphthyl-1-yl)vinyl-1-yl, 2,2-bis(diphenyl-1-yl)vinyl-1-yl, a stilbenyl group, a styrenyl group, and the like, but are not limited thereto.

In the present specification, a cycloalkyl group is not particularly limited, but the number of carbon atoms thereof is preferably 3 to 60. According to one embodiment, the number of carbon atoms of the cycloalkyl group is 3 to 30. According to another embodiment, the number of carbon atoms of the cycloalkyl group is 3 to 20. According to still another embodiment, the number of carbon atoms of the cycloalkyl group is 3 to 6. Specific examples thereof include cyclopropyl, cyclobutyl, cyclopentyl, 3-methylcyclopentyl, 2,3-dimethylcyclopentyl, cyclohexyl, 3-methylcyclohexyl, 4-methylcyclohexyl, 2,3-dimethylcyclohexyl, 3,4,5-trimethylcyclohexyl, 4-tert-butylcyclohexyl, cycloheptyl, cyclooctyl, and the like, but are not limited thereto.

In the present specification, an aryl group is not particularly limited, but preferably has 6 to 60 carbon atoms, and can be a monocyclic aryl group or a polycyclic aryl group. According to one embodiment, the number of carbon atoms of the aryl group is 6 to 30. According to one embodiment, the number of carbon atoms of the aryl group is 6 to 20. The aryl group can be a phenyl group, a biphenyl group, a terphenyl group, or the like as the monocyclic aryl group, but is not limited thereto. Examples of the polycyclic aryl group include a naphthyl group, an anthracenyl group, a phenanthryl group, a pyrenyl group, a perylenyl group, a chrysenyl group, a fluorenyl group, and the like, but are not limited thereto.

In the present specification, a fluorenyl group can be substituted, and two substituent groups can be bonded to each other to form a spiro structure. In the case where the fluorenyl group is substituted,

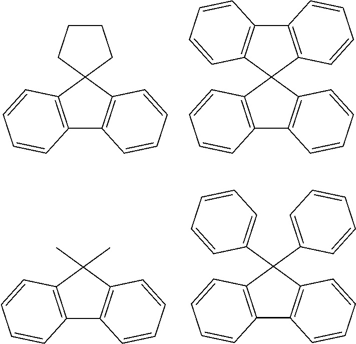

and the like can be formed. However, the structure is not limited thereto.

In the present specification, a heterocyclic group is a heterocyclic group including one or more of O, N, Si, and S as a heteroatom, and the number of carbon atoms thereof is not particularly limited, but is preferably 2 to 60. Examples of the heterocyclic group include a thiophene group, a furan group, a pyrrole group, an imidazole group, a thiazole group, an oxazole group, an oxadiazole group, a triazole group, a pyridyl group, a bipyridyl group, a pyrimidyl group, a triazine group, an acridyl group, a pyridazine group, a pyrazinyl group, a quinolinyl group, a quinazoline group, a quinoxalinyl group, a phthalazinyl group, a pyridopyrimidinyl group, a pyridopyrazinyl group, a pyrazinopyrazinyl group, an isoquinoline group, an indole group, a carbazole group, a benzoxazole group, a benzimidazole group, a benzothiazole group, a benzocarbazole group, a benzothiophene group, a dibenzothiophene group, a benzofuranyl group, a phenanthroline group, an isoxazolyl group, a thiadiazolyl group, a phenothiazinyl group, a dibenzofuranyl group, and the like, but are not limited thereto.

In the present specification, the aryl group in the aralkyl group, the aralkenyl group, the alkylaryl group, and the arylamine group is the same as the aforementioned examples of the aryl group. In the present specification, the alkyl group in the aralkyl group, the alkylaryl group, and the alkylamine group is the same as the aforementioned examples of the alkyl group. In the present specification, the heteroaryl in the heteroarylamine can be applied to the aforementioned description of the heterocyclic group. In the present specification, the alkenyl group in the aralkenyl group is the same as the aforementioned examples of the alkenyl group. In the present specification, the aforementioned description of the aryl group can be applied except that the arylene is a divalent group. In the present specification, the aforementioned description of the heterocyclic group can be applied except that the heteroarylene is a divalent group. In the present specification, the aforementioned description of the aryl group or cycloalkyl group can be applied except that the hydrocarbon ring is not a monovalent group but is formed by combining two substituent groups. In the present specification, the aforementioned description of the heterocyclic group can be applied, except that the heterocycle is not a monovalent group but is formed by combining two substituent groups.

In Chemical Formula 1, depending on the structure fused to a carbazole group and an indene group via A, Chemical Formula 1 can be represented by the following Chemical Formulae 1-1, 1-2, 1-3, 1-4, 1-5, or 1-6.

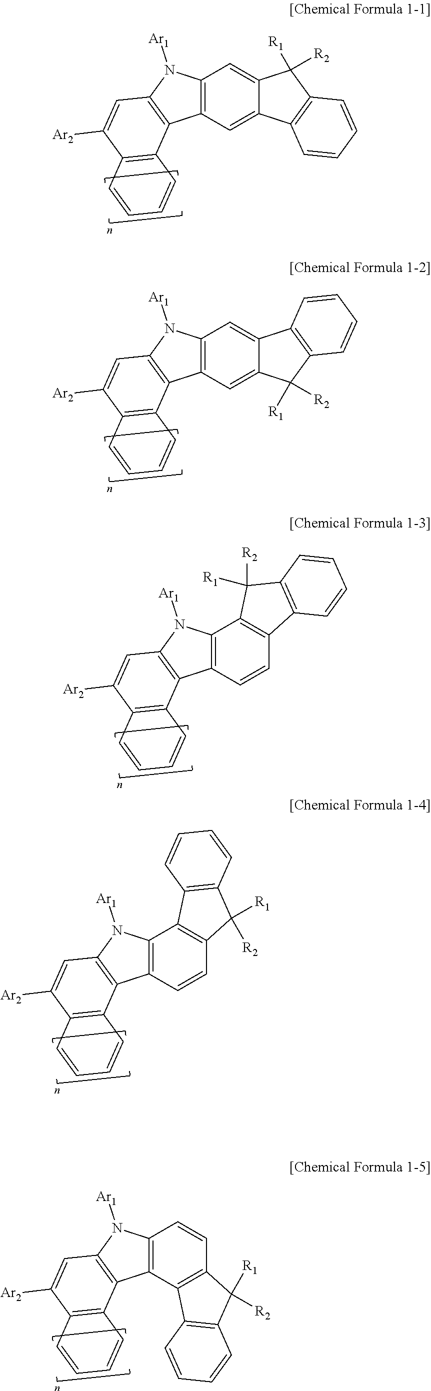

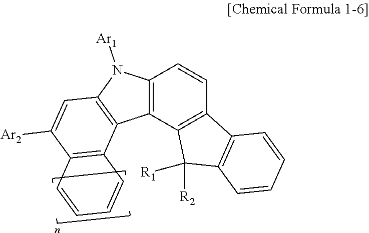

In Chemical Formulae 1-1, 1-2, 1-3, 1-4, 1-5, and 1-6, R₁, R₂, Ar₁, Ar₂, and n are as defined in Chemical Formula 1.

Preferably, R₁ and R₂ are methyl or phenyl.

Preferably, Ar₃ and Ar₄ are phenyl, biphenyl, terphenyl, or dimethylfluorenyl.

Representative examples of the compound represented by Chemical Formula 1 are as follows.

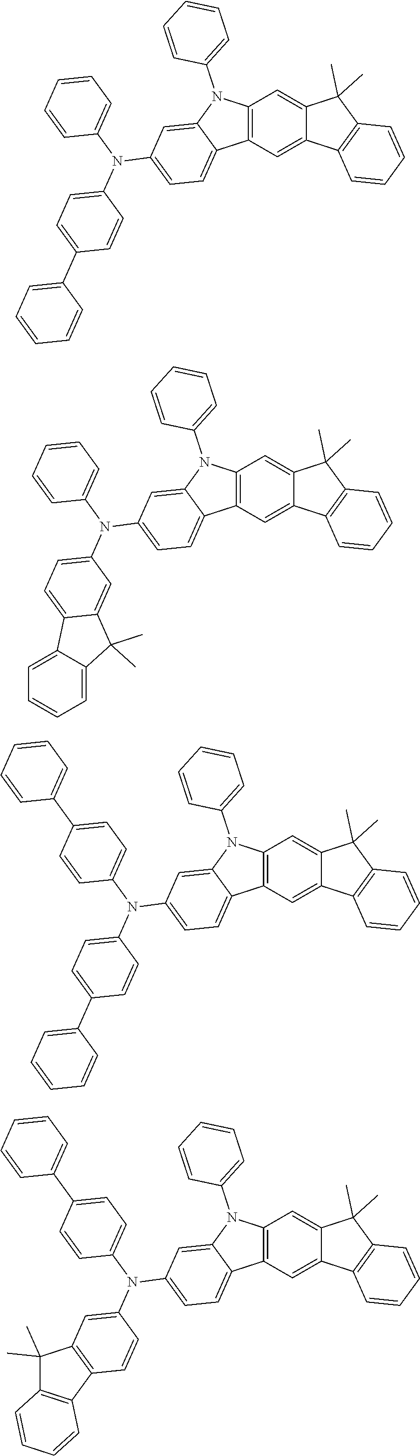

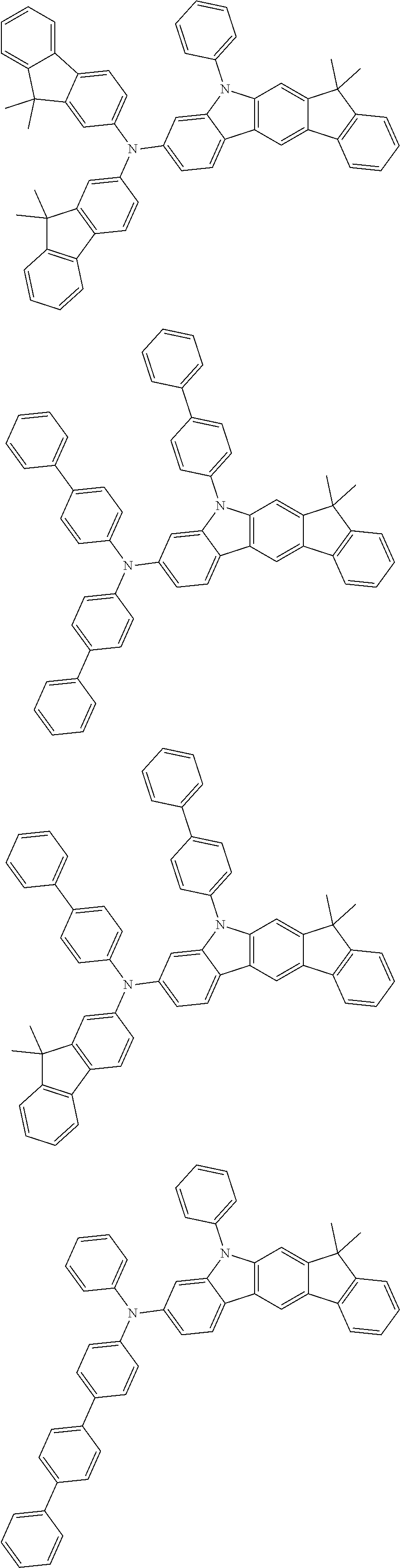 [SMILES: CC1(C)c2ccccc2-c2ccc(N(c3ccc4c(c3)C(C)(C)c3ccccc3-4)c3ccc4c5cc6c(cc5n(-c5ccccc5)c4c3)C(C)(C)c3ccccc3-6)cc21 ; CC1(C)c2ccccc2-c2cc3c4ccc(N(c5ccc(-c6ccccc6)cc5)c5ccc(-c6ccccc6)cc5)cc4n(-c4ccc(-c5ccccc5)cc4)c3cc21 ; CC1(C)c2ccccc2-c2ccc(N(c3ccc(-c4ccccc4)cc3)c3ccc4c5cc6c(cc5n(-c5ccc(-c7ccccc7)cc5)c4c3)C(C)(C)c3ccccc3-6)cc21 ; CC1(C)c2ccccc2-c2cc3c4ccc(N(c5ccccc5)c5ccc(-c6ccc(-c7ccccc7)cc6)cc5)cc4n(-c4ccccc4)c3cc21]

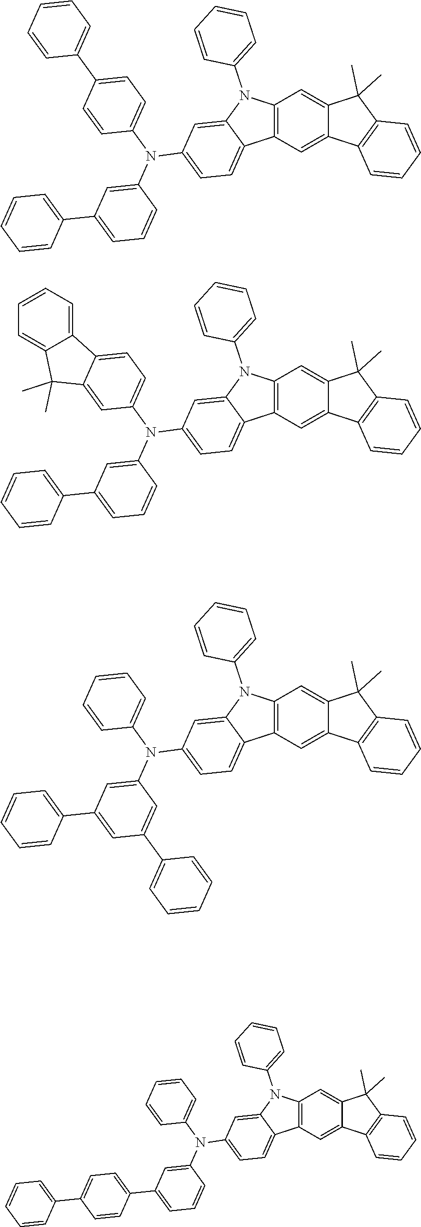

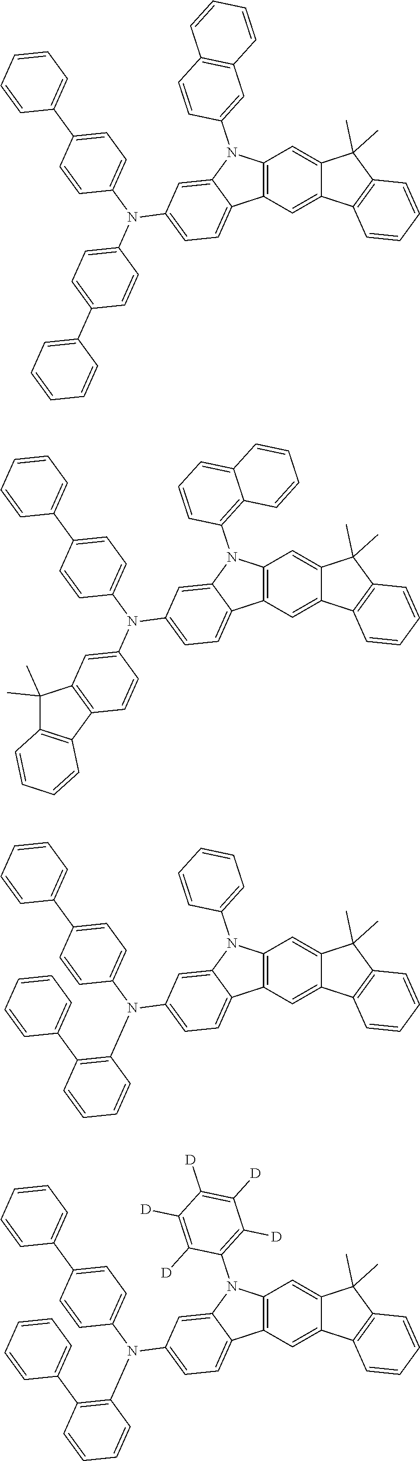 [SMILES: CC1(C)c2ccccc2-c2cc3c4ccc(N(c5ccc(-c6ccccc6)cc5)c5ccc(-c6ccccc6)cc5)cc4n(-c4ccc5ccccc5c4)c3cc21 ; CC1(C)c2ccccc2-c2ccc(N(c3ccc(-c4ccccc4)cc3)c3ccc4c5cc6c(cc5n(-c5cccc7ccccc57)c4c3)C(C)(C)c3ccccc3-6)cc21 ; CC1(C)c2ccccc2-c2cc3c4ccc(N(c5ccc(-c6ccccc6)cc5)c5ccccc5-c5ccccc5)cc4n(-c4ccccc4)c3cc21 ; [2H]c1c([2H])c([2H])c(-n2c3cc(N(c4ccc(-c5ccccc5)cc4)c4ccccc4-c4ccccc4)ccc3c3cc4c(cc32)C(C)(C)c2ccccc2-4)c([2H])c1[2H]]

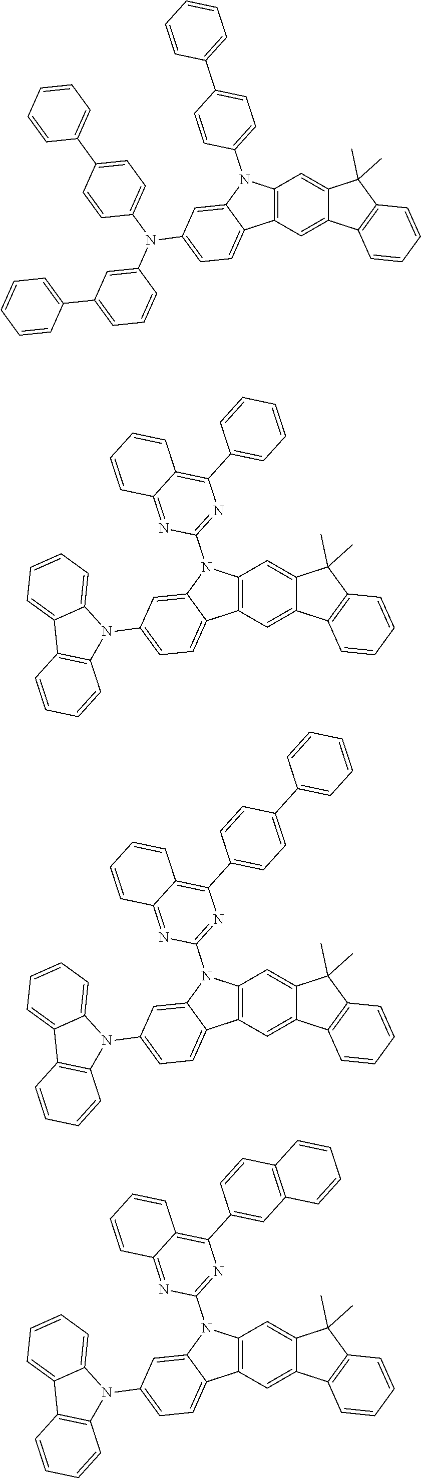 [SMILES: CC1(C)c2ccccc2-c2cc3c4ccc(N(c5ccc(-c6ccccc6)cc5)c5cccc(-c6ccccc6)c5)cc4n(-c4ccc(-c5ccccc5)cc4)c3cc21 ; CC1(C)c2ccccc2-c2cc3c4ccc(-n5c6ccccc6c6ccccc65)cc4n(-c4nc(-c5ccccc5)c5ccccc5n4)c3cc21 ; CC1(C)c2ccccc2-c2cc3c4ccc(-n5c6ccccc6c6ccccc65)cc4n(-c4nc(-c5ccc(-c6ccccc6)cc5)c5ccccc5n4)c3cc21 ; CC1(C)c2ccccc2-c2cc3c4ccc(-n5c6ccccc6c6ccccc65)cc4n(-c4nc(-c5ccc6ccccc6c5)c5ccccc5n4)c3cc21]

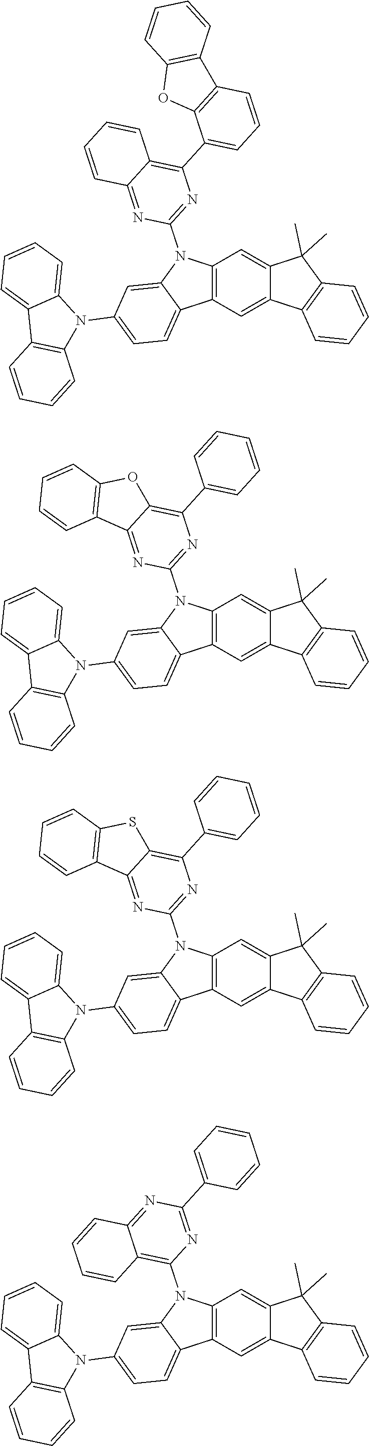 [SMILES: CC1(C)c2ccccc2-c2cc3c4ccc(-n5c6ccccc6c6ccccc65)cc4n(-c4nc(-c5cccc6c5oc5ccccc56)c5ccccc5n4)c3cc21 ; CC1(C)c2ccccc2-c2cc3c4ccc(-n5c6ccccc6c6ccccc65)cc4n(-c4nc(-c5ccccc5)c5oc6ccccc6c5n4)c3cc21 ; CC1(C)c2ccccc2-c2cc3c4ccc(-n5c6ccccc6c6ccccc65)cc4n(-c4nc(-c5ccccc5)c5sc6ccccc6c5n4)c3cc21 ; CC1(C)c2ccccc2-c2cc3c4ccc(-n5c6ccccc6c6ccccc65)cc4n(-c4nc(-c5ccccc5)nc5ccccc45)c3cc21]

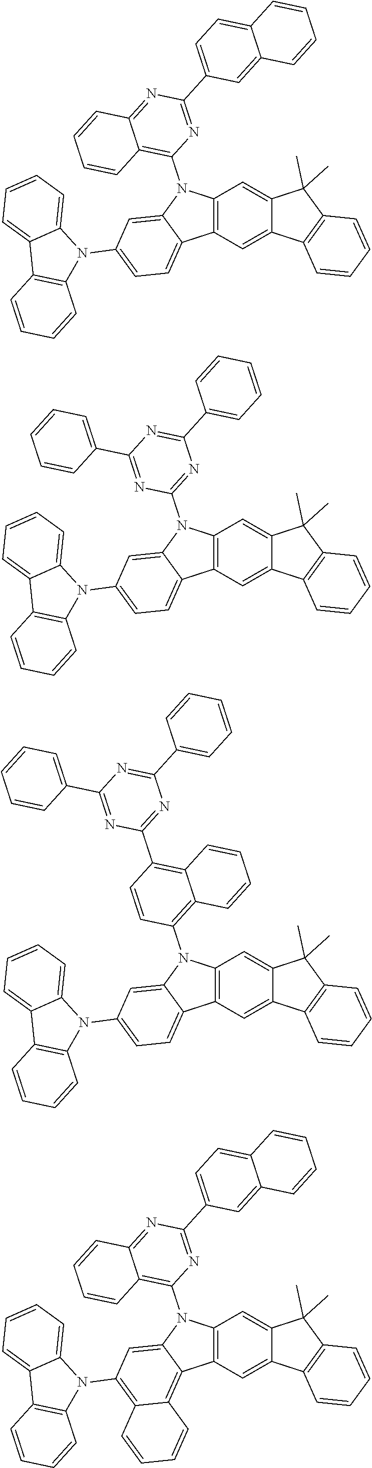 [SMILES: CC1(C)c2ccccc2-c2cc3c4ccc(-n5c6ccccc6c6ccccc65)cc4n(-c4nc(-c5ccc6ccccc6c5)nc5ccccc45)c3cc21 ; CC1(C)c2ccccc2-c2cc3c4ccc(-n5c6ccccc6c6ccccc65)cc4n(-c4nc(-c5ccccc5)nc(-c5ccccc5)n4)c3cc21 ; CC1(C)c2ccccc2-c2cc3c4ccc(-n5c6ccccc6c6ccccc65)cc4n(-c4ccc(-c5nc(-c6ccccc6)nc(-c6ccccc6)n5)c5ccccc45)c3cc21 ; CC1(C)c2ccccc2-c2cc3c4c5ccccc5c(-n5c6ccccc6c6ccccc65)cc4n(-c4nc(-c5ccc6ccccc6c5)nc5ccccc45)c3cc21]

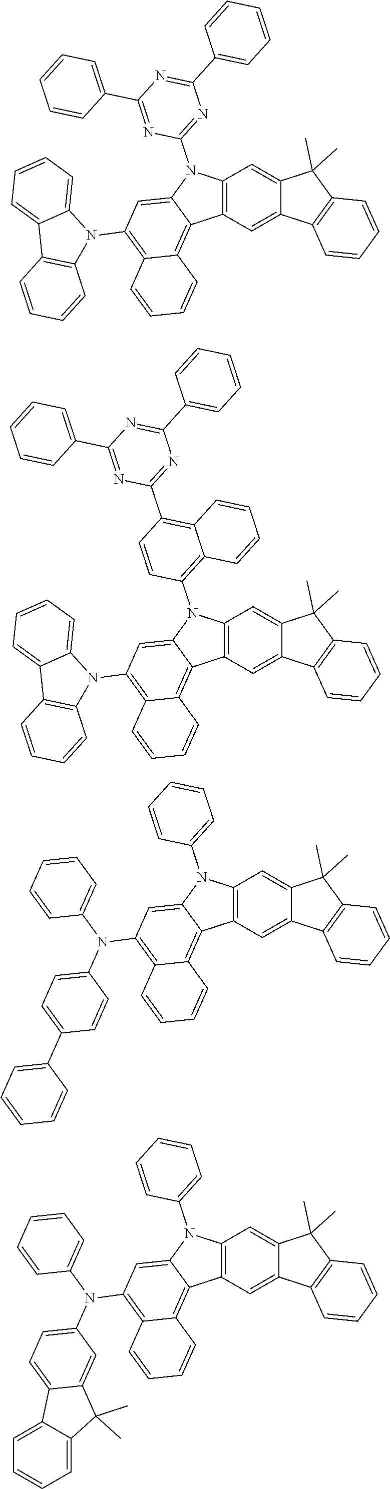

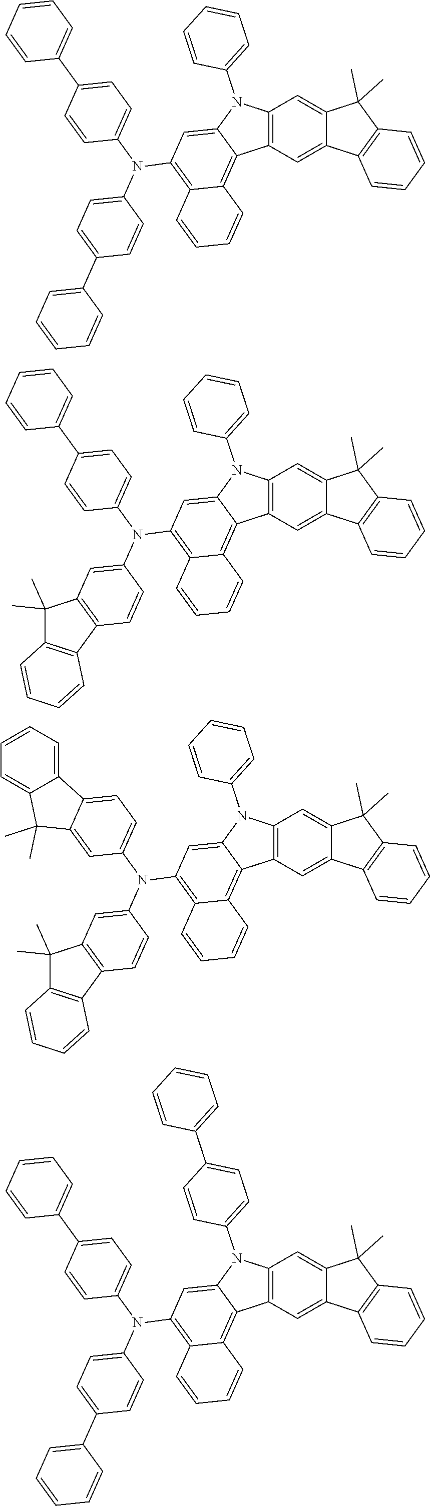

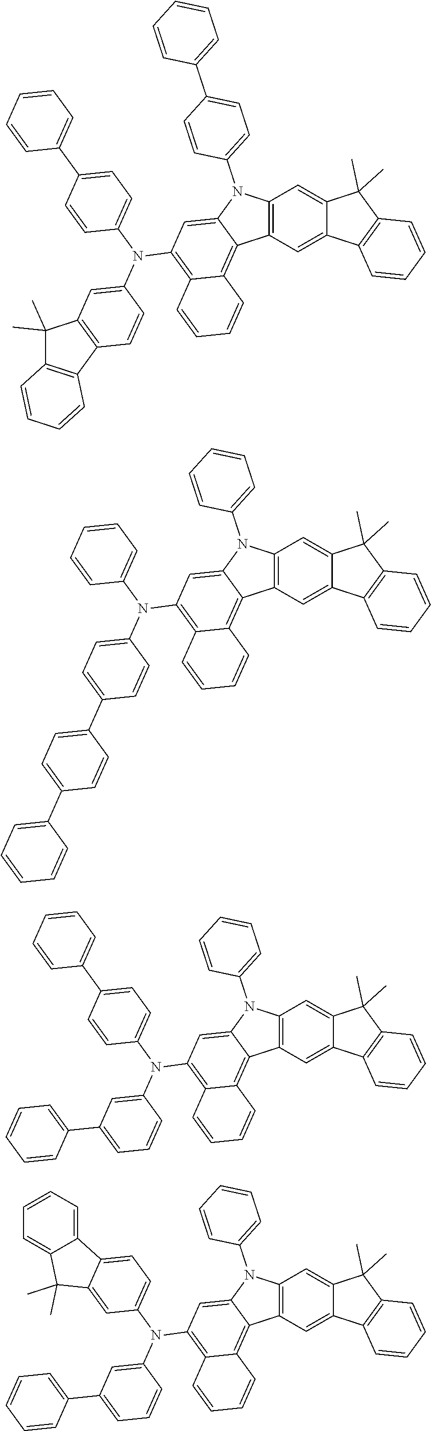

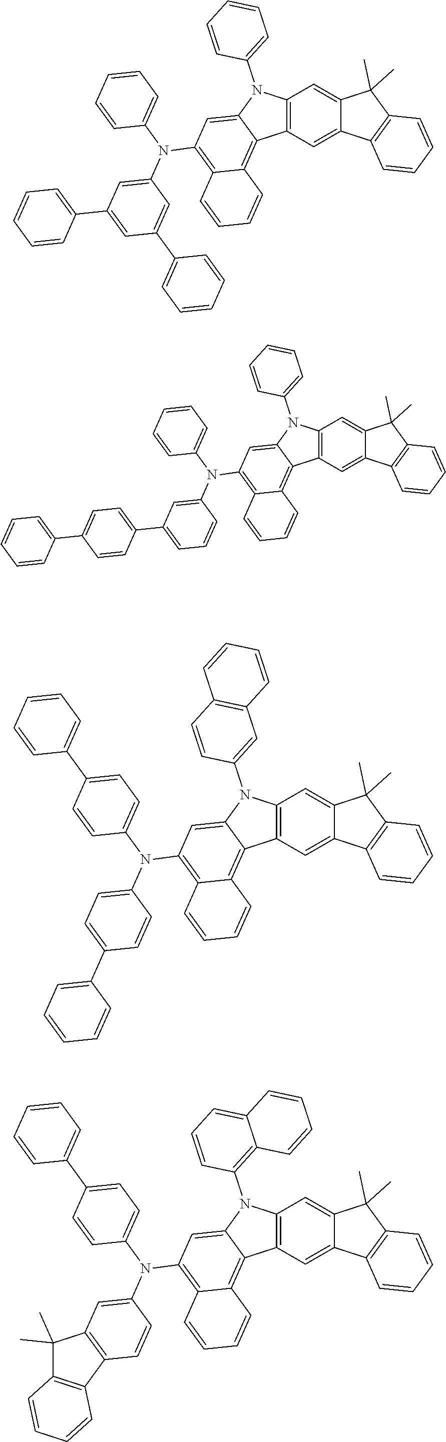 [SMILES: CC1(C)c2ccccc2-c2cc3c4c5ccccc5c(N(c5ccccc5)c5cc(-c6ccccc6)cc(-c6ccccc6)c5)cc4n(-c4ccccc4)c3cc21 ; CC1(C)c2ccccc2-c2cc3c4c5ccccc5c(N(c5ccccc5)c5cccc(-c6ccc(-c7ccccc7)cc6)c5)cc4n(-c4ccccc4)c3cc21 ; CC1(C)c2ccccc2-c2cc3c4c5ccccc5c(N(c5ccc(-c6ccccc6)cc5)c5ccc(-c6ccccc6)cc5)cc4n(-c4ccc5ccccc5c4)c3cc21 ; CC1(C)c2ccccc2-c2ccc(N(c3ccc(-c4ccccc4)cc3)c3cc4c(c5ccccc35)c3cc5c(cc3n4-c3cccc4ccccc34)C(C)(C)c3ccccc3-5)cc21]

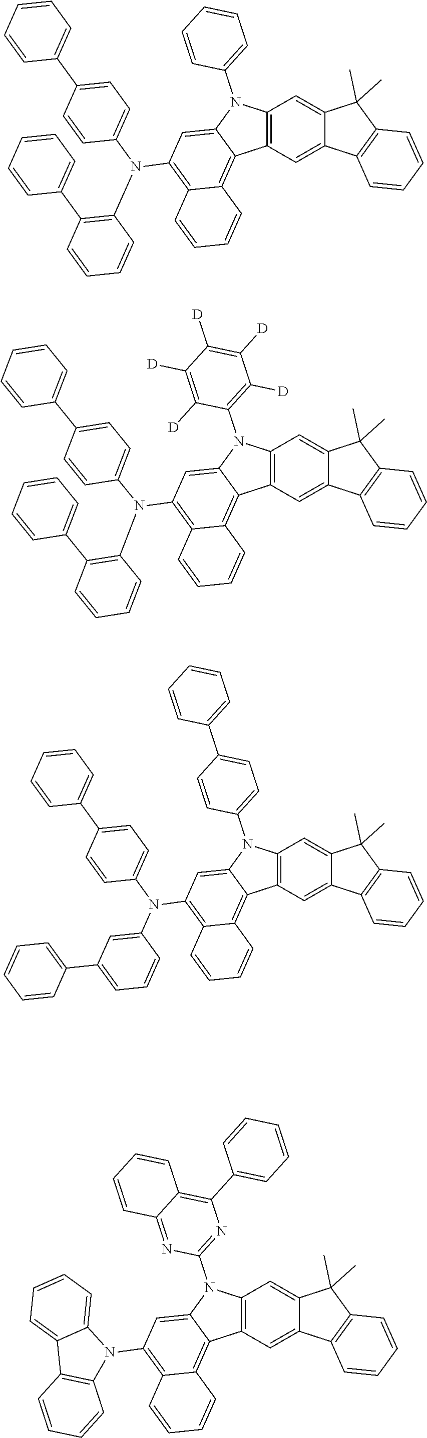 [SMILES: CC1(C)c2ccccc2-c2cc3c4c5ccccc5c(N(c5ccc(-c6ccccc6)cc5)c5ccccc5-c5ccccc5)cc4n(-c4ccccc4)c3cc21 ; [2H]c1c([2H])c([2H])c(-n2c3cc4c(cc3c3c5ccccc5c(N(c5ccc(-c6ccccc6)cc5)c5ccccc5-c5ccccc5)cc32)-c2ccccc2C4(C)C)c([2H])c1[2H] ; CC1(C)c2ccccc2-c2cc3c4c5ccccc5c(N(c5ccc(-c6ccccc6)cc5)c5cccc(-c6ccccc6)c5)cc4n(-c4ccc(-c5ccccc5)cc4)c3cc21 ; CC1(C)c2ccccc2-c2cc3c4c5ccccc5c(-n5c6ccccc6c6ccccc65)cc4n(-c4nc(-c5ccccc5)c5ccccc5n4)c3cc21]

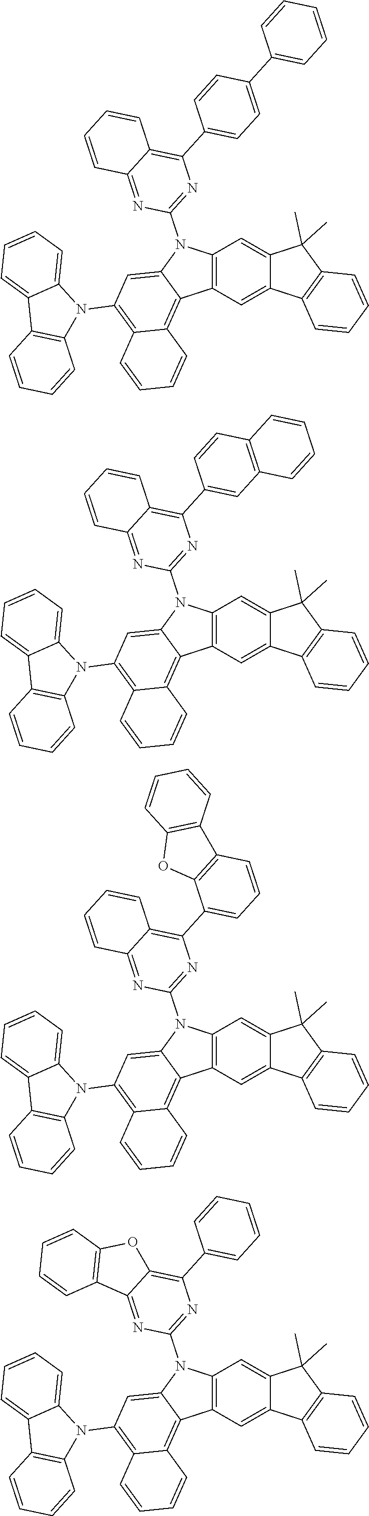

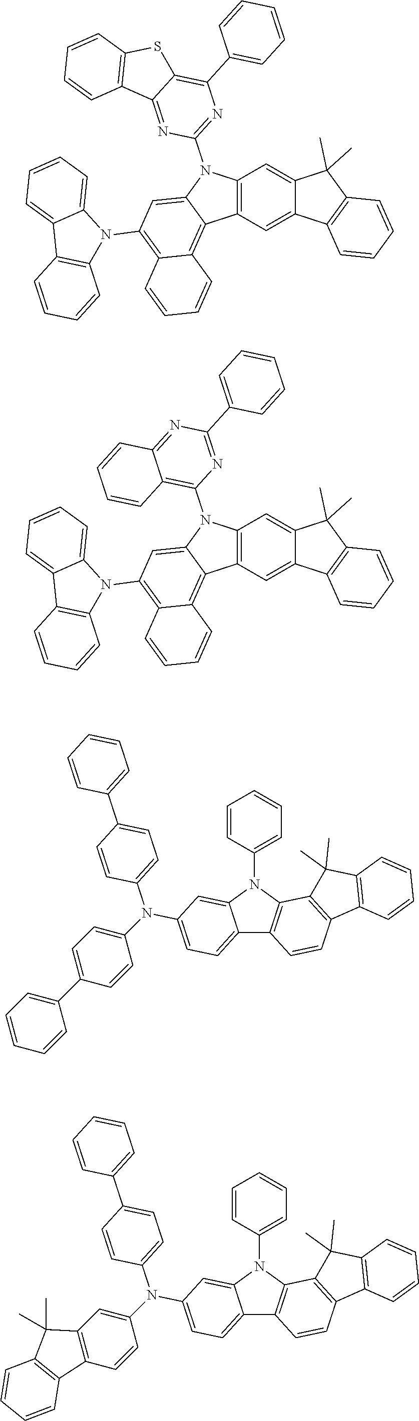 [SMILES: CC1(C)c2ccccc2-c2cc3c4c5ccccc5c(-n5c6ccccc6c6ccccc65)cc4n(-c4nc(-c5ccccc5)c5sc6ccccc6c5n4)c3cc21 ; CC1(C)c2ccccc2-c2cc3c4c5ccccc5c(-n5c6ccccc6c6ccccc65)cc4n(-c4nc(-c5ccccc5)nc5ccccc45)c3cc21 ; CC1(C)c2ccccc2-c2ccc3c4ccc(N(c5ccc(-c6ccccc6)cc5)c5ccc(-c6ccccc6)cc5)cc4n(-c4ccccc4)c3c21 ; CC1(C)c2ccccc2-c2ccc(N(c3ccc(-c4ccccc4)cc3)c3ccc4c5ccc6c(c5n(-c5ccccc5)c4c3)C(C)(C)c3ccccc3-6)cc21]

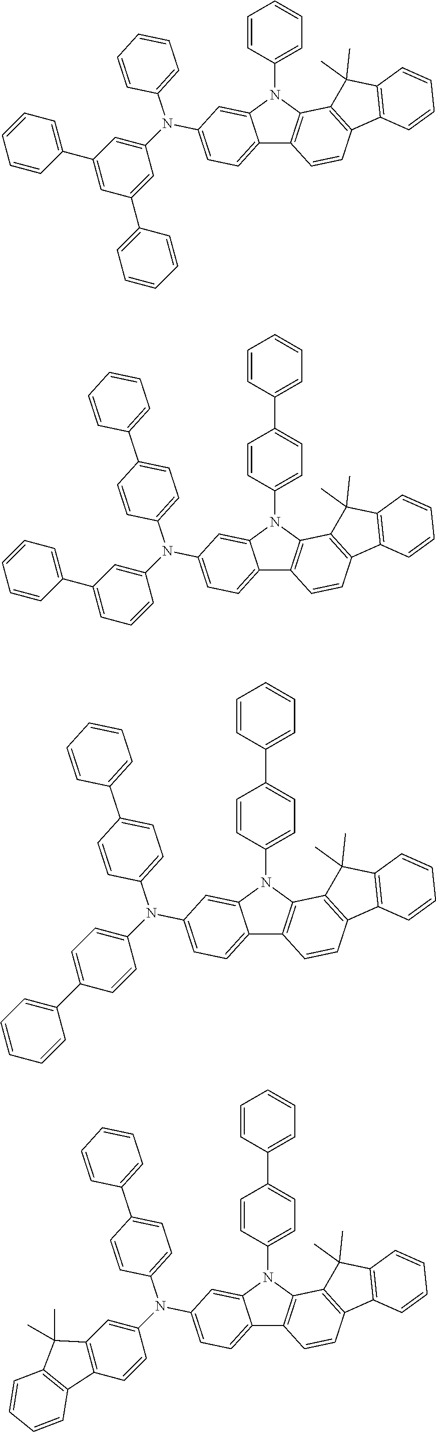 [SMILES: CC1(C)c2ccccc2-c2ccc3c4ccc(N(c5ccccc5)c5cc(-c6ccccc6)cc(-c6ccccc6)c5)cc4n(-c4ccccc4)c3c21 ; CC1(C)c2ccccc2-c2ccc3c4ccc(N(c5ccc(-c6ccccc6)cc5)c5cccc(-c6ccccc6)c5)cc4n(-c4ccc(-c5ccccc5)cc4)c3c21 ; CC1(C)c2ccccc2-c2ccc3c4ccc(N(c5ccc(-c6ccccc6)cc5)c5ccc(-c6ccccc6)cc5)cc4n(-c4ccc(-c5ccccc5)cc4)c3c21 ; CC1(C)c2ccccc2-c2ccc(N(c3ccc(-c4ccccc4)cc3)c3ccc4c5ccc6c(c5n(-c5ccc(-c7ccccc7)cc5)c4c3)C(C)(C)c3ccccc3-6)cc21]

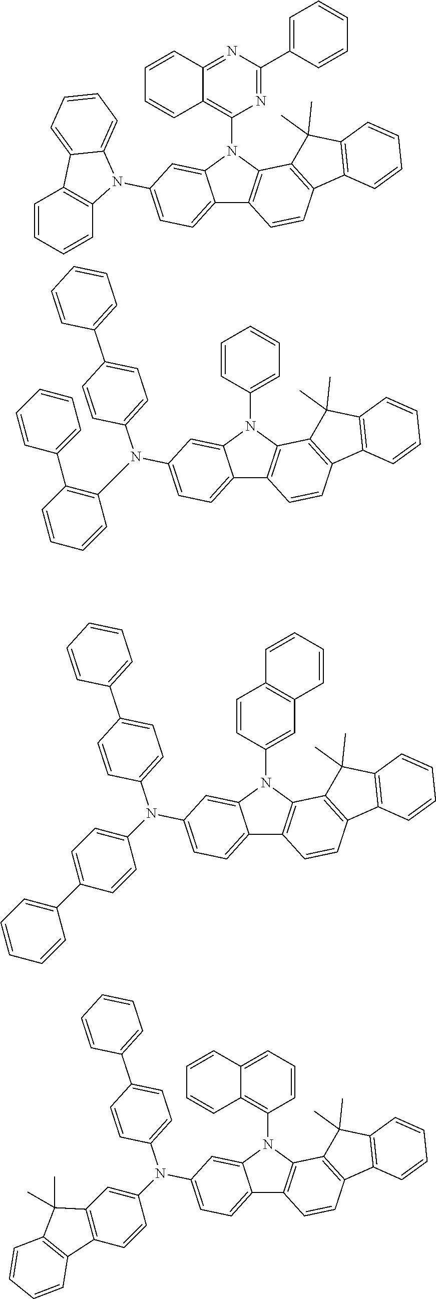

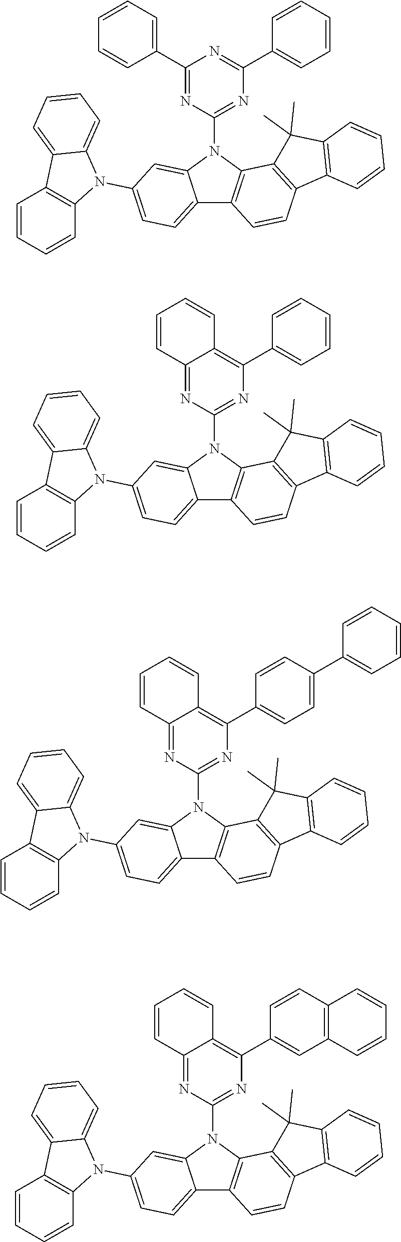 [SMILES: CC1(C)c2ccccc2-c2ccc3c4ccc(-n5c6ccccc6c6ccccc65)cc4n(-c4nc(-c5ccccc5)nc(-c5ccccc5)n4)c3c21 ; CC1(C)c2ccccc2-c2ccc3c4ccc(-n5c6ccccc6c6ccccc65)cc4n(-c4nc(-c5ccccc5)c5ccccc5n4)c3c21 ; CC1(C)c2ccccc2-c2ccc3c4ccc(-n5c6ccccc6c6ccccc65)cc4n(-c4nc(-c5ccc(-c6ccccc6)cc5)c5ccccc5n4)c3c21 ; CC1(C)c2ccccc2-c2ccc3c4ccc(-n5c6ccccc6c6ccccc65)cc4n(-c4nc(-c5ccc6ccccc6c5)c5ccccc5n4)c3c21]

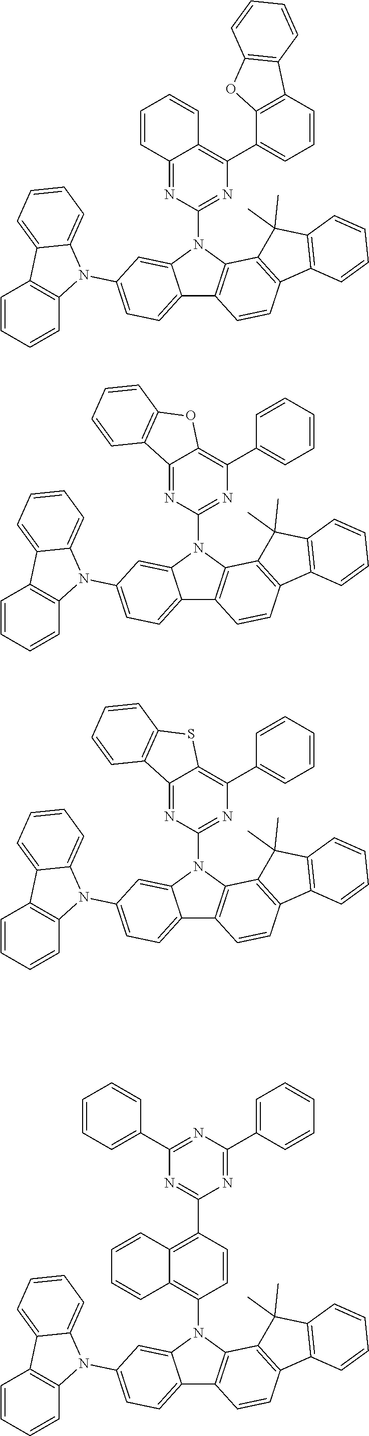 [SMILES: CC1(C)c2ccccc2-c2ccc3c4ccc(-n5c6ccccc6c6ccccc65)cc4n(-c4nc(-c5cccc6c5oc5ccccc56)c5ccccc5n4)c3c21 ; CC1(C)c2ccccc2-c2ccc3c4ccc(-n5c6ccccc6c6ccccc65)cc4n(-c4nc(-c5ccccc5)c5oc6ccccc6c5n4)c3c21 ; CC1(C)c2ccccc2-c2ccc3c4ccc(-n5c6ccccc6c6ccccc65)cc4n(-c4nc(-c5ccccc5)c5sc6ccccc6c5n4)c3c21 ; CC1(C)c2ccccc2-c2ccc3c4ccc(-n5c6ccccc6c6ccccc65)cc4n(-c4ccc(-c5nc(-c6ccccc6)nc(-c6ccccc6)n5)c5ccccc45)c3c21]

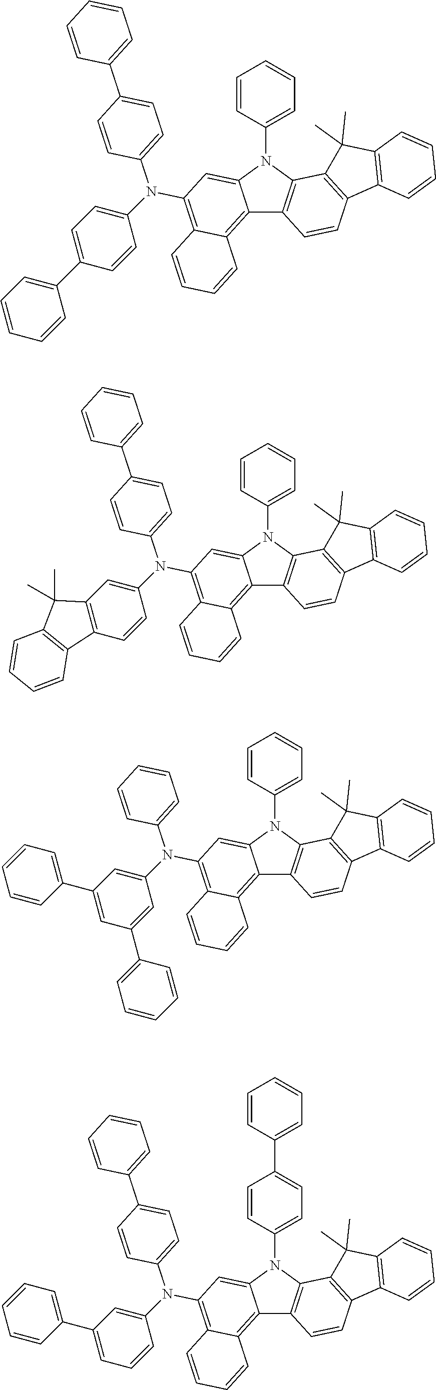 [SMILES: CC1(C)c2ccccc2-c2ccc3c4c5ccccc5c(N(c5ccc(-c6ccccc6)cc5)c5ccc(-c6ccccc6)cc5)cc4n(-c4ccccc4)c3c21 ; CC1(C)c2ccccc2-c2ccc(N(c3ccc(-c4ccccc4)cc3)c3cc4c(c5ccccc35)c3ccc5c(c3n4-c3ccccc3)C(C)(C)c3ccccc3-5)cc21 ; CC1(C)c2ccccc2-c2ccc3c4c5ccccc5c(N(c5ccccc5)c5cc(-c6ccccc6)cc(-c6ccccc6)c5)cc4n(-c4ccccc4)c3c21 ; CC1(C)c2ccccc2-c2ccc3c4c5ccccc5c(N(c5ccc(-c6ccccc6)cc5)c5cccc(-c6ccccc6)c5)cc4n(-c4ccc(-c5ccccc5)cc4)c3c21]

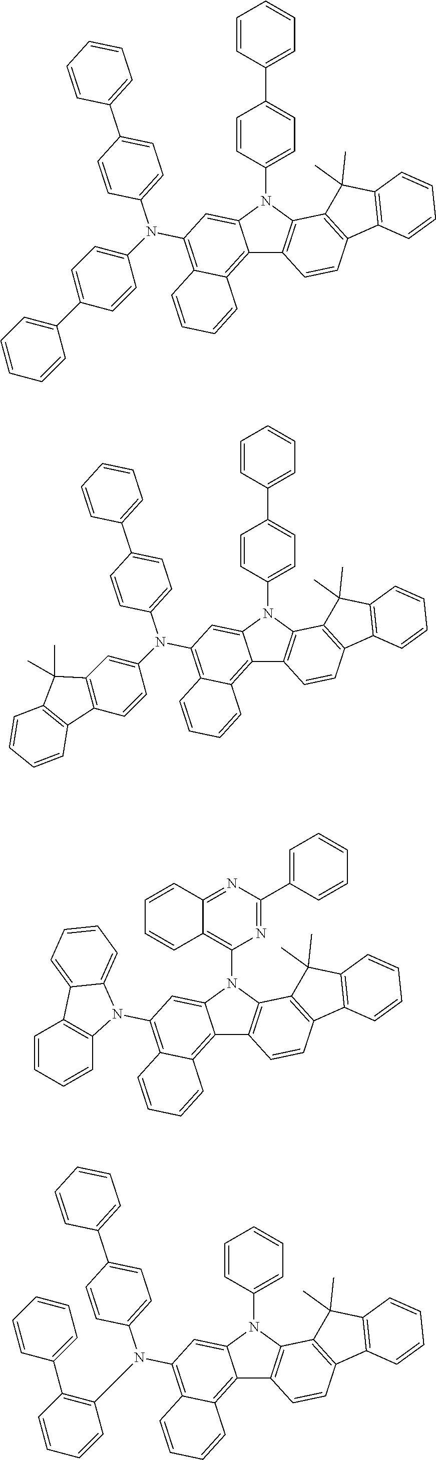 [SMILES: CC1(C)c2ccccc2-c2ccc3c4c5ccccc5c(N(c5ccc(-c6ccccc6)cc5)c5ccc(-c6ccccc6)cc5)cc4n(-c4ccc(-c5ccccc5)cc4)c3c21 ; CC1(C)c2ccccc2-c2ccc(N(c3ccc(-c4ccccc4)cc3)c3cc4c(c5ccccc35)c3ccc5c(c3n4-c3ccc(-c4ccccc4)cc3)C(C)(C)c3ccccc3-5)cc21 ; CC1(C)c2ccccc2-c2ccc3c4c5ccccc5c(-n5c6ccccc6c6ccccc65)cc4n(-c4nc(-c5ccccc5)nc5ccccc45)c3c21 ; CC1(C)c2ccccc2-c2ccc3c4c5ccccc5c(N(c5ccc(-c6ccccc6)cc5)c5ccccc5-c5ccccc5)cc4n(-c4ccccc4)c3c21]

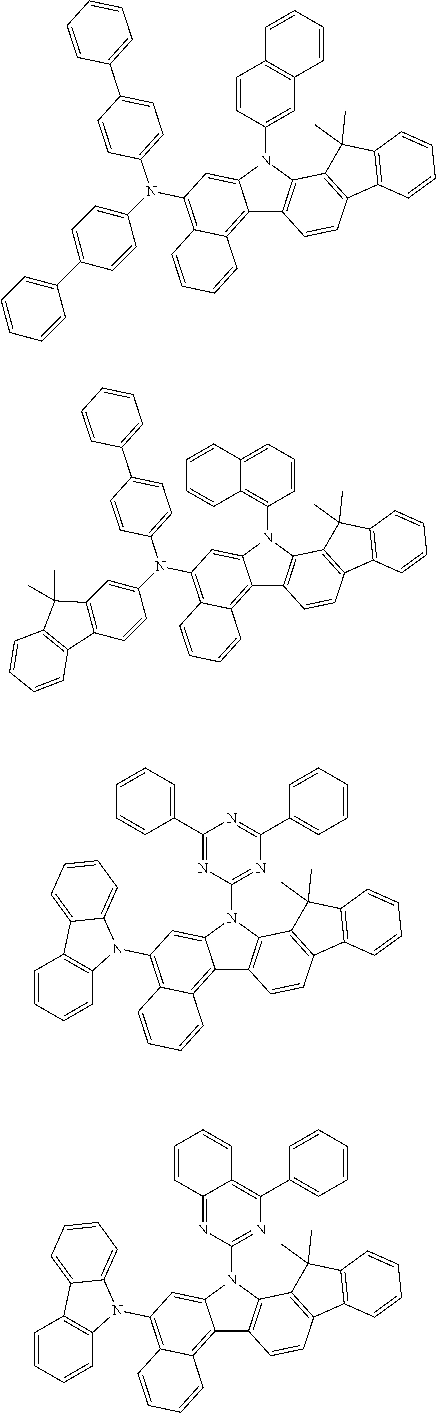 [SMILES: CC1(C)c2ccccc2-c2ccc3c4c5ccccc5c(N(c5ccc(-c6ccccc6)cc5)c5ccc(-c6ccccc6)cc5)cc4n(-c4ccc5ccccc5c4)c3c21 ; CC1(C)c2ccccc2-c2ccc(N(c3ccc(-c4ccccc4)cc3)c3cc4c(c5ccccc35)c3ccc5c(c3n4-c3cccc4ccccc34)C(C)(C)c3ccccc3-5)cc21 ; CC1(C)c2ccccc2-c2ccc3c4c5ccccc5c(-n5c6ccccc6c6ccccc65)cc4n(-c4nc(-c5ccccc5)nc(-c5ccccc5)n4)c3c21 ; CC1(C)c2ccccc2-c2ccc3c4c5ccccc5c(-n5c6ccccc6c6ccccc65)cc4n(-c4nc(-c5ccccc5)c5ccccc5n4)c3c21]

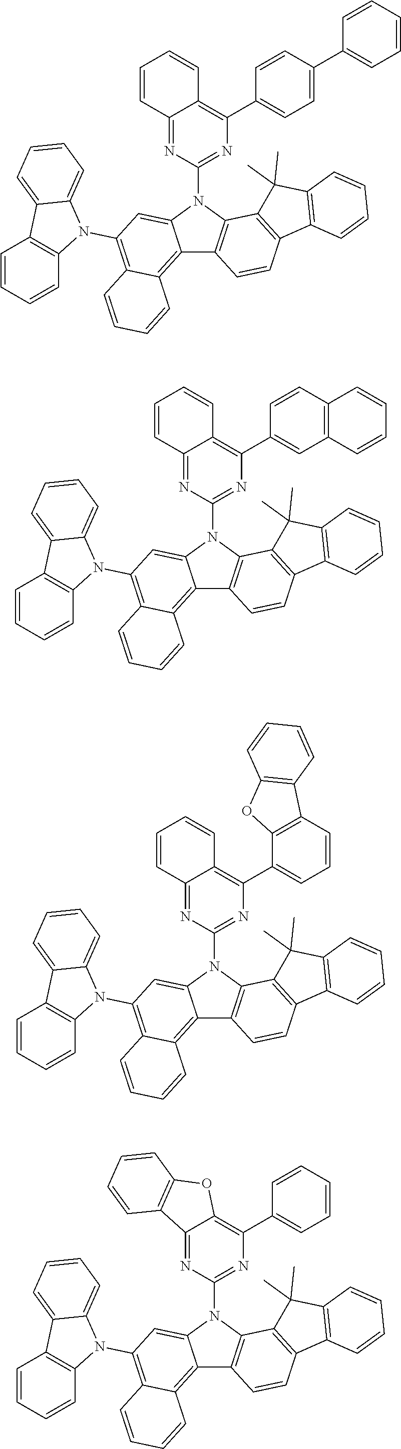 [SMILES: CC1(C)c2ccccc2-c2ccc3c4c5ccccc5c(-n5c6ccccc6c6ccccc65)cc4n(-c4nc(-c5ccc(-c6ccccc6)cc5)c5ccccc5n4)c3c21 ; CC1(C)c2ccccc2-c2ccc3c4c5ccccc5c(-n5c6ccccc6c6ccccc65)cc4n(-c4nc(-c5ccc6ccccc6c5)c5ccccc5n4)c3c21 ; CC1(C)c2ccccc2-c2ccc3c4c5ccccc5c(-n5c6ccccc6c6ccccc65)cc4n(-c4nc(-c5cccc6c5oc5ccccc56)c5ccccc5n4)c3c21 ; CC1(C)c2ccccc2-c2ccc3c4c5ccccc5c(-n5c6ccccc6c6ccccc65)cc4n(-c4nc(-c5ccccc5)c5oc6ccccc6c5n4)c3c21]

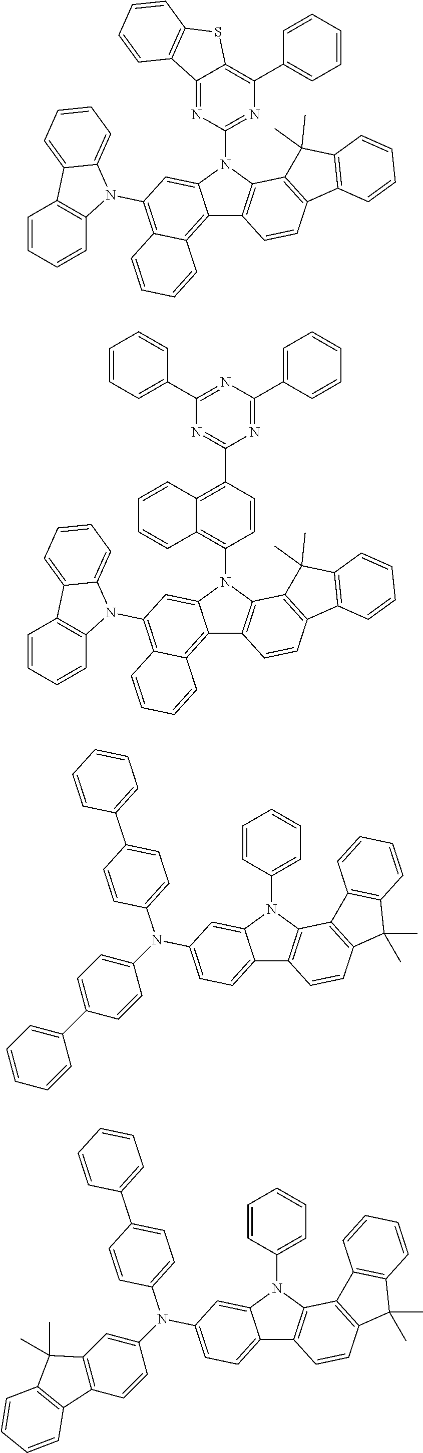

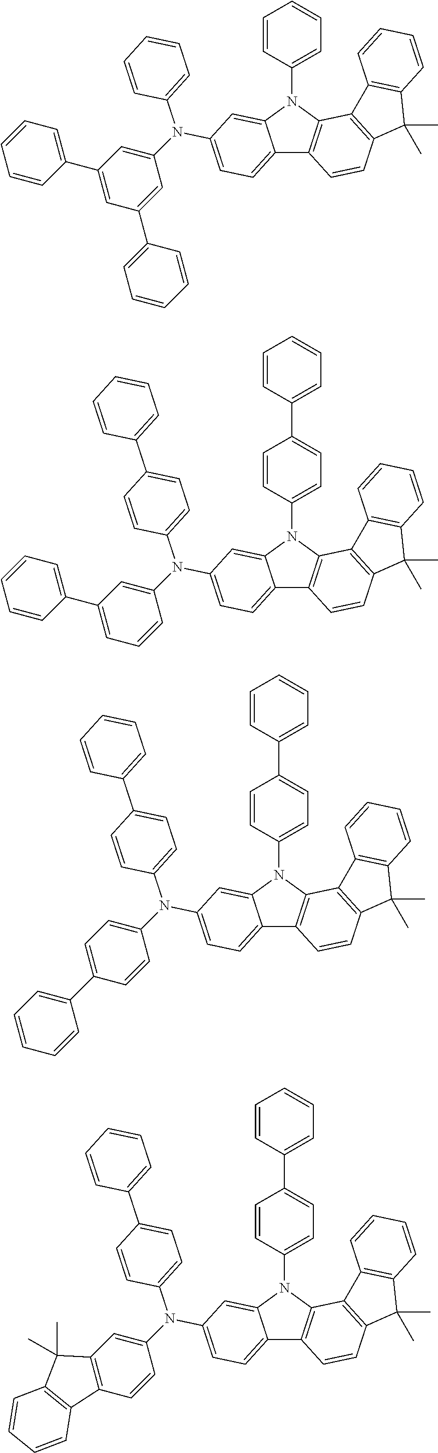 [SMILES: CC1(C)c2ccccc2-c2c1ccc1c3ccc(N(c4ccccc4)c4cc(-c5ccccc5)cc(-c5ccccc5)c4)cc3n(-c3ccccc3)c21 ; CC1(C)c2ccccc2-c2c1ccc1c3ccc(N(c4ccc(-c5ccccc5)cc4)c4cccc(-c5ccccc5)c4)cc3n(-c3ccc(-c4ccccc4)cc3)c21 ; CC1(C)c2ccccc2-c2c1ccc1c3ccc(N(c4ccc(-c5ccccc5)cc4)c4ccc(-c5ccccc5)cc4)cc3n(-c3ccc(-c4ccccc4)cc3)c21 ; CC1(C)c2ccccc2-c2ccc(N(c3ccc(-c4ccccc4)cc3)c3ccc4c5ccc6c(c5n(-c5ccc(-c7ccccc7)cc5)c4c3)-c3ccccc3C6(C)C)cc21]

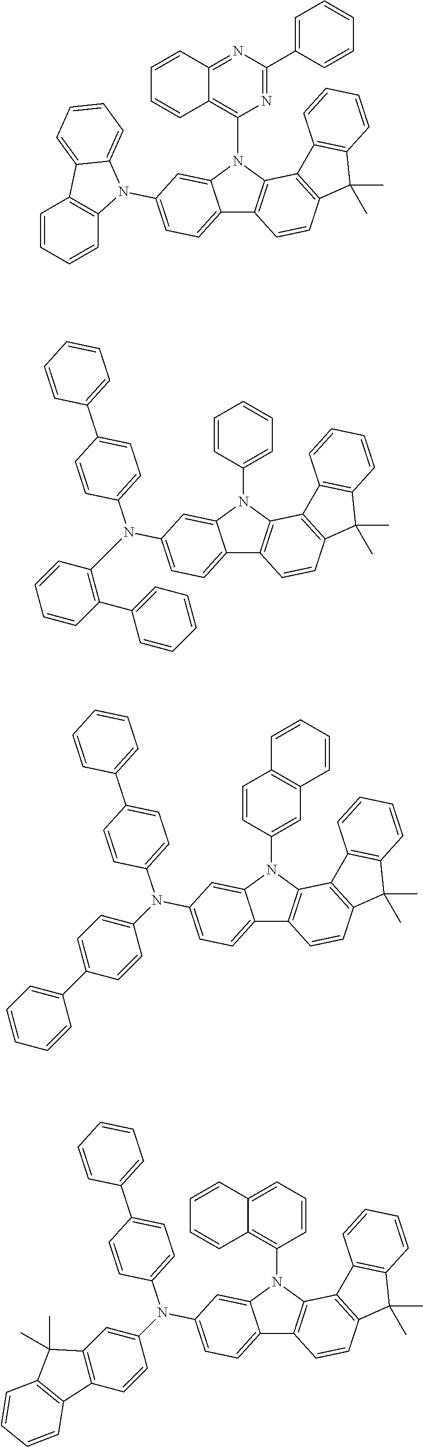 [SMILES: CC1(C)c2ccccc2-c2c1ccc1c3ccc(-n4c5ccccc5c5ccccc54)cc3n(-c3nc(-c4ccccc4)nc4ccccc34)c21 ; CC1(C)c2ccccc2-c2c1ccc1c3ccc(N(c4ccc(-c5ccccc5)cc4)c4ccccc4-c4ccccc4)cc3n(-c3ccccc3)c21 ; CC1(C)c2ccccc2-c2c1ccc1c3ccc(N(c4ccc(-c5ccccc5)cc4)c4ccc(-c5ccccc5)cc4)cc3n(-c3ccc4ccccc4c3)c21 ; CC1(C)c2ccccc2-c2ccc(N(c3ccc(-c4ccccc4)cc3)c3ccc4c5ccc6c(c5n(-c5cccc7ccccc57)c4c3)-c3ccccc3C6(C)C)cc21]

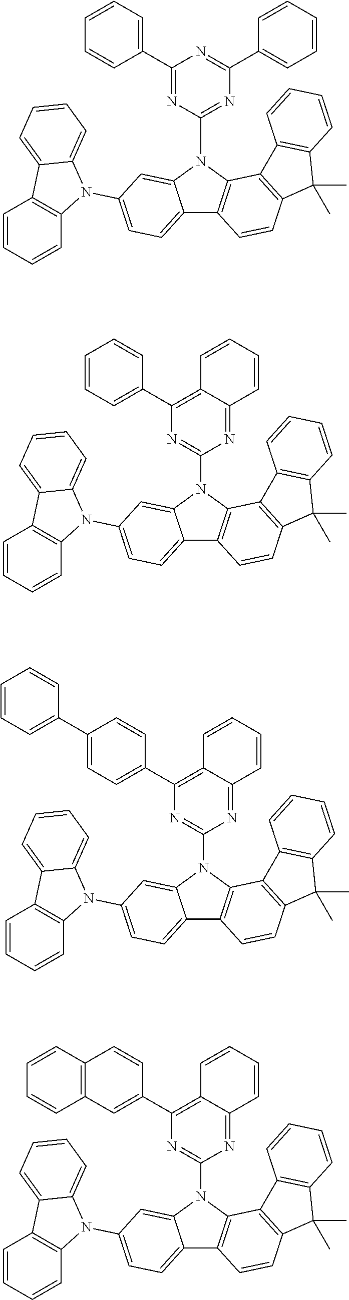 [SMILES: CC1(C)c2ccccc2-c2c1ccc1c3ccc(-n4c5ccccc5c5ccccc54)cc3n(-c3nc(-c4ccccc4)nc(-c4ccccc4)n3)c21 ; CC1(C)c2ccccc2-c2c1ccc1c3ccc(-n4c5ccccc5c5ccccc54)cc3n(-c3nc(-c4ccccc4)c4ccccc4n3)c21 ; CC1(C)c2ccccc2-c2c1ccc1c3ccc(-n4c5ccccc5c5ccccc54)cc3n(-c3nc(-c4ccc(-c5ccccc5)cc4)c4ccccc4n3)c21 ; CC1(C)c2ccccc2-c2c1ccc1c3ccc(-n4c5ccccc5c5ccccc54)cc3n(-c3nc(-c4ccc5ccccc5c4)c4ccccc4n3)c21]

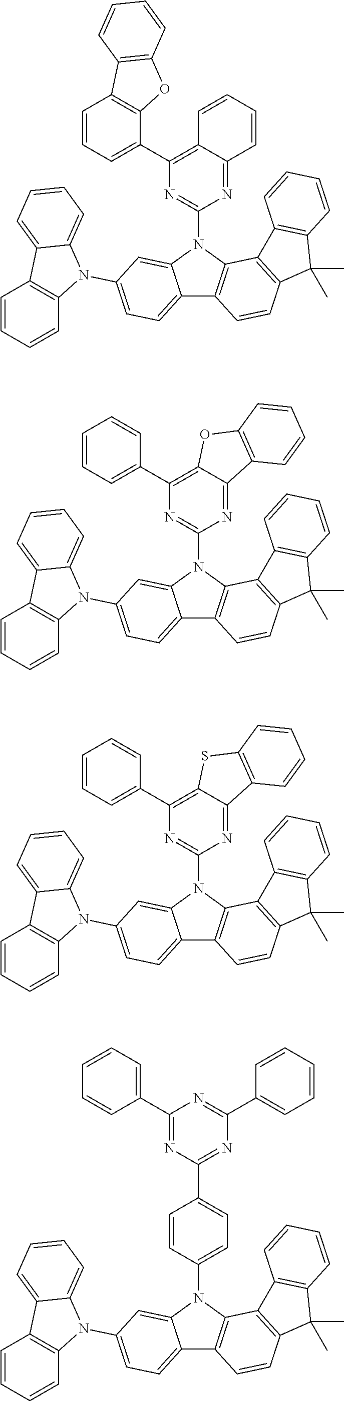 [SMILES: CC1(C)c2ccccc2-c2c1ccc1c3ccc(-n4c5ccccc5c5ccccc54)cc3n(-c3nc(-c4cccc5c4oc4ccccc45)c4ccccc4n3)c21 ; CC1(C)c2ccccc2-c2c1ccc1c3ccc(-n4c5ccccc5c5ccccc54)cc3n(-c3nc(-c4ccccc4)c4oc5ccccc5c4n3)c21 ; CC1(C)c2ccccc2-c2c1ccc1c3ccc(-n4c5ccccc5c5ccccc54)cc3n(-c3nc(-c4ccccc4)c4sc5ccccc5c4n3)c21 ; CC1(C)c2ccccc2-c2c1ccc1c3ccc(-n4c5ccccc5c5ccccc54)cc3n(-c3ccc(-c4nc(-c5ccccc5)nc(-c5ccccc5)n4)cc3)c21]

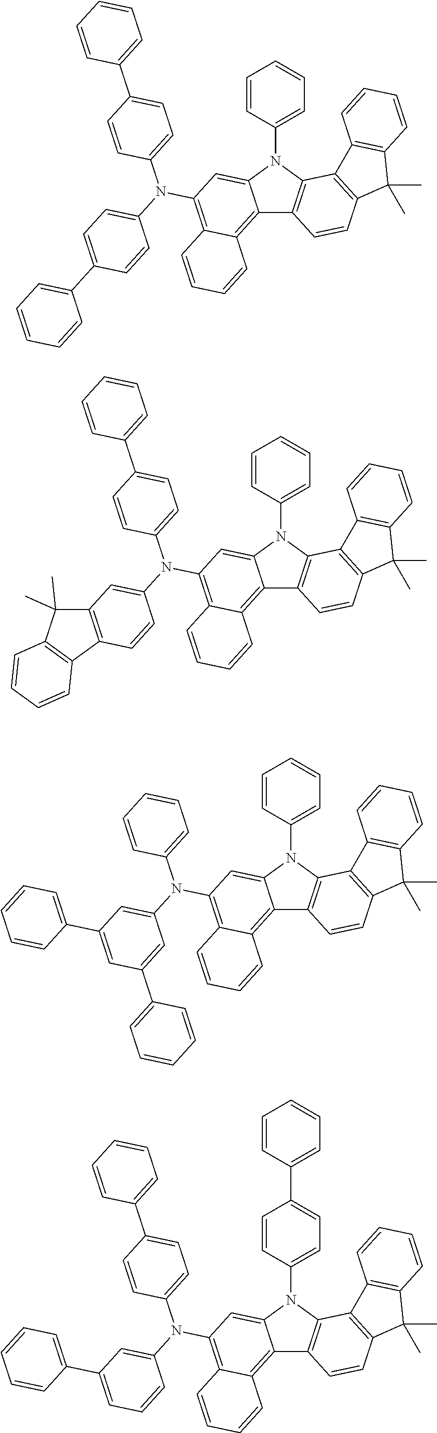 [SMILES: CC1(C)c2ccccc2-c2c1ccc1c3c4ccccc4c(N(c4ccc(-c5ccccc5)cc4)c4ccc(-c5ccccc5)cc4)cc3n(-c3ccccc3)c21 ; CC1(C)c2ccccc2-c2ccc(N(c3ccc(-c4ccccc4)cc3)c3cc4c(c5ccccc35)c3ccc5c(c3n4-c3ccccc3)-c3ccccc3C5(C)C)cc21 ; CC1(C)c2ccccc2-c2c1ccc1c3c4ccccc4c(N(c4ccccc4)c4cc(-c5ccccc5)cc(-c5ccccc5)c4)cc3n(-c3ccccc3)c21 ; CC1(C)c2ccccc2-c2c1ccc1c3c4ccccc4c(N(c4ccc(-c5ccccc5)cc4)c4cccc(-c5ccccc5)c4)cc3n(-c3ccc(-c4ccccc4)cc3)c21]

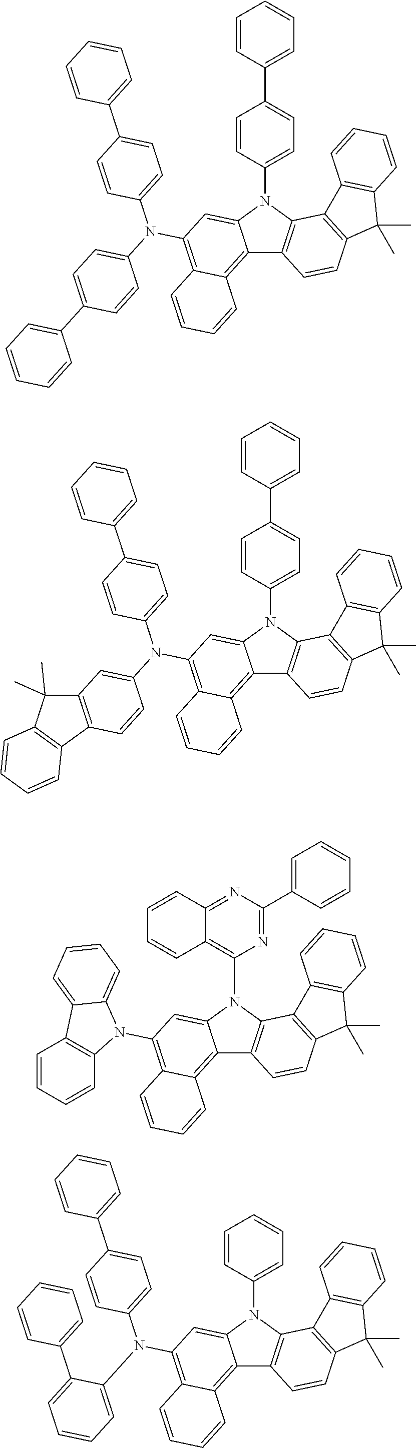 [SMILES: CC1(C)c2ccccc2-c2c1ccc1c3c4ccccc4c(N(c4ccc(-c5ccccc5)cc4)c4ccc(-c5ccccc5)cc4)cc3n(-c3ccc(-c4ccccc4)cc3)c21 ; CC1(C)c2ccccc2-c2ccc(N(c3ccc(-c4ccccc4)cc3)c3cc4c(c5ccccc35)c3ccc5c(c3n4-c3ccc(-c4ccccc4)cc3)-c3ccccc3C5(C)C)cc21 ; CC1(C)c2ccccc2-c2c1ccc1c3c4ccccc4c(-n4c5ccccc5c5ccccc54)cc3n(-c3nc(-c4ccccc4)nc4ccccc34)c21 ; CC1(C)c2ccccc2-c2c1ccc1c3c4ccccc4c(N(c4ccc(-c5ccccc5)cc4)c4ccccc4-c4ccccc4)cc3n(-c3ccccc3)c21]

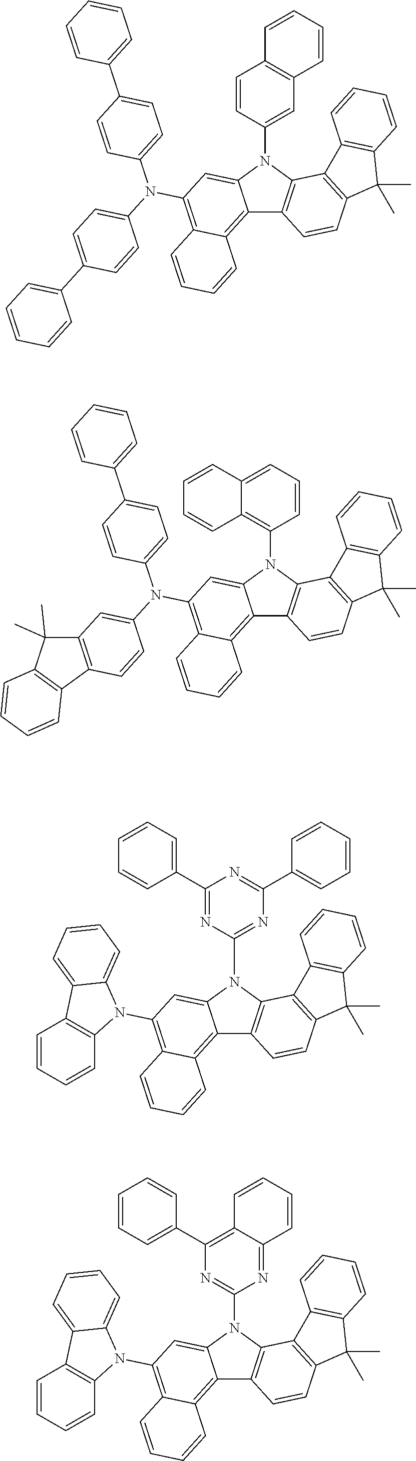 [SMILES: CC1(C)c2ccccc2-c2c1ccc1c3c4ccccc4c(N(c4ccc(-c5ccccc5)cc4)c4ccc(-c5ccccc5)cc4)cc3n(-c3ccc4ccccc4c3)c21 ; CC1(C)c2ccccc2-c2ccc(N(c3ccc(-c4ccccc4)cc3)c3cc4c(c5ccccc35)c3ccc5c(c3n4-c3cccc4ccccc34)-c3ccccc3C5(C)C)cc21 ; CC1(C)c2ccccc2-c2c1ccc1c3c4ccccc4c(-n4c5ccccc5c5ccccc54)cc3n(-c3nc(-c4ccccc4)nc(-c4ccccc4)n3)c21 ; CC1(C)c2ccccc2-c2c1ccc1c3c4ccccc4c(-n4c5ccccc5c5ccccc54)cc3n(-c3nc(-c4ccccc4)c4ccccc4n3)c21]

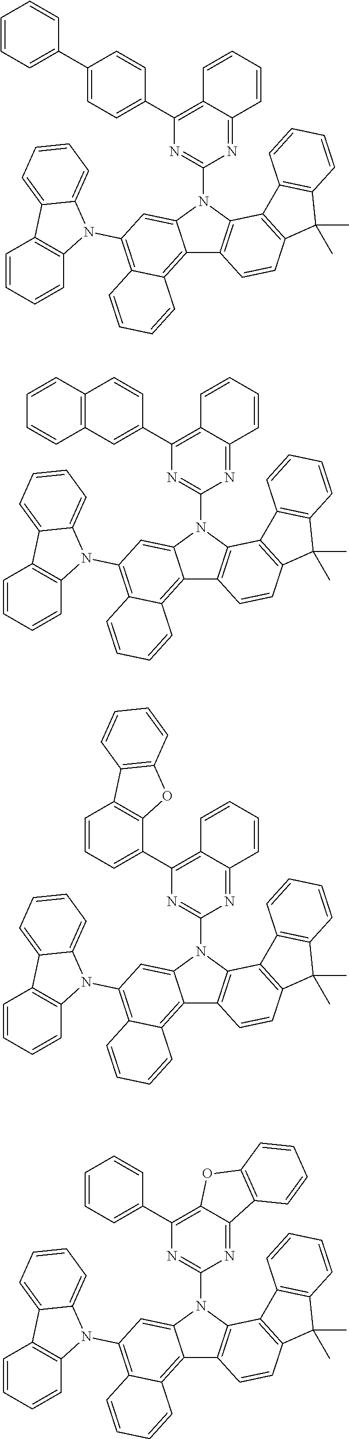 [SMILES: CC1(C)c2ccccc2-c2c1ccc1c3c4ccccc4c(-n4c5ccccc5c5ccccc54)cc3n(-c3nc(-c4ccc(-c5ccccc5)cc4)c4ccccc4n3)c21 ; CC1(C)c2ccccc2-c2c1ccc1c3c4ccccc4c(-n4c5ccccc5c5ccccc54)cc3n(-c3nc(-c4ccc5ccccc5c4)c4ccccc4n3)c21 ; CC1(C)c2ccccc2-c2c1ccc1c3c4ccccc4c(-n4c5ccccc5c5ccccc54)cc3n(-c3nc(-c4cccc5c4oc4ccccc45)c4ccccc4n3)c21 ; CC1(C)c2ccccc2-c2c1ccc1c3c4ccccc4c(-n4c5ccccc5c5ccccc54)cc3n(-c3nc(-c4ccccc4)c4oc5ccccc5c4n3)c21]

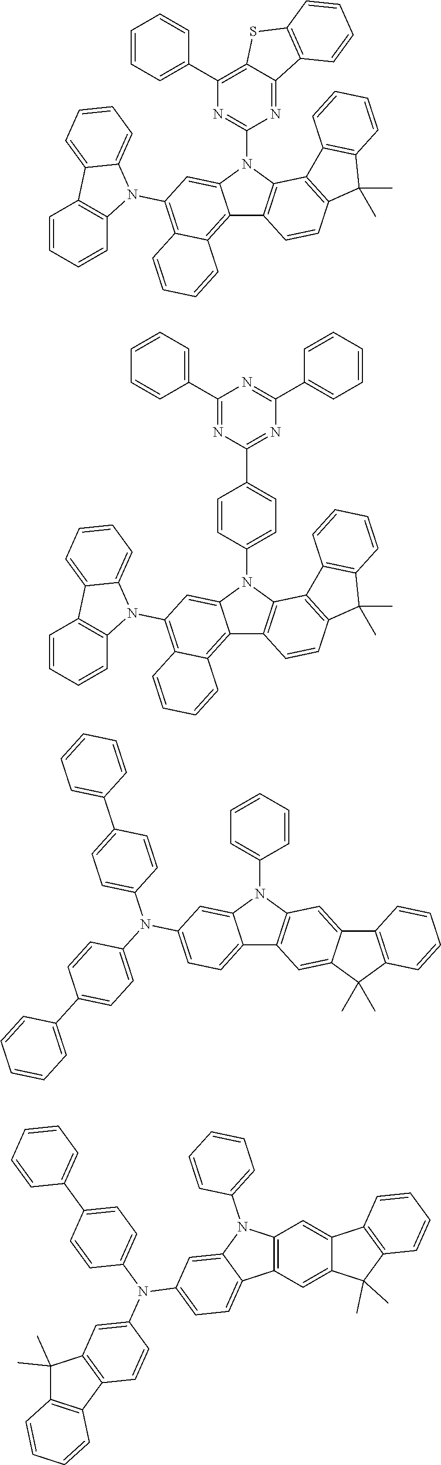

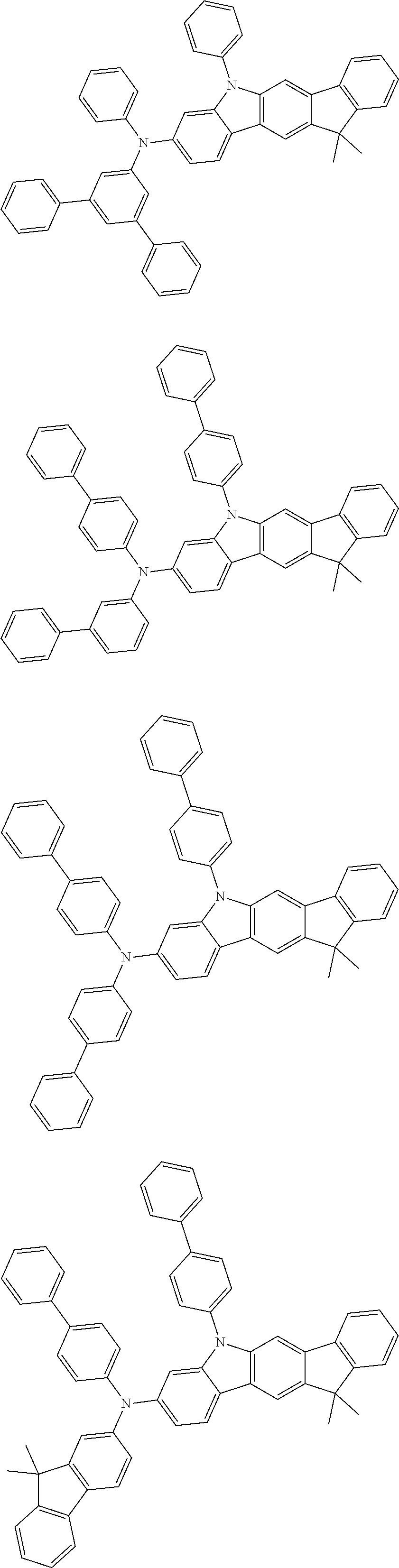 [SMILES: CC1(C)c2ccccc2-c2cc3c(cc21)c1ccc(N(c2ccccc2)c2cc(-c4ccccc4)cc(-c4ccccc4)c2)cc1n3-c1ccccc1 ; CC1(C)c2ccccc2-c2cc3c(cc21)c1ccc(N(c2ccc(-c4ccccc4)cc2)c2cccc(-c4ccccc4)c2)cc1n3-c1ccc(-c2ccccc2)cc1 ; CC1(C)c2ccccc2-c2cc3c(cc21)c1ccc(N(c2ccc(-c4ccccc4)cc2)c2ccc(-c4ccccc4)cc2)cc1n3-c1ccc(-c2ccccc2)cc1 ; CC1(C)c2ccccc2-c2ccc(N(c3ccc(-c4ccccc4)cc3)c3ccc4c5cc6c(cc5n(-c5ccc(-c7ccccc7)cc5)c4c3)-c3ccccc3C6(C)C)cc21]

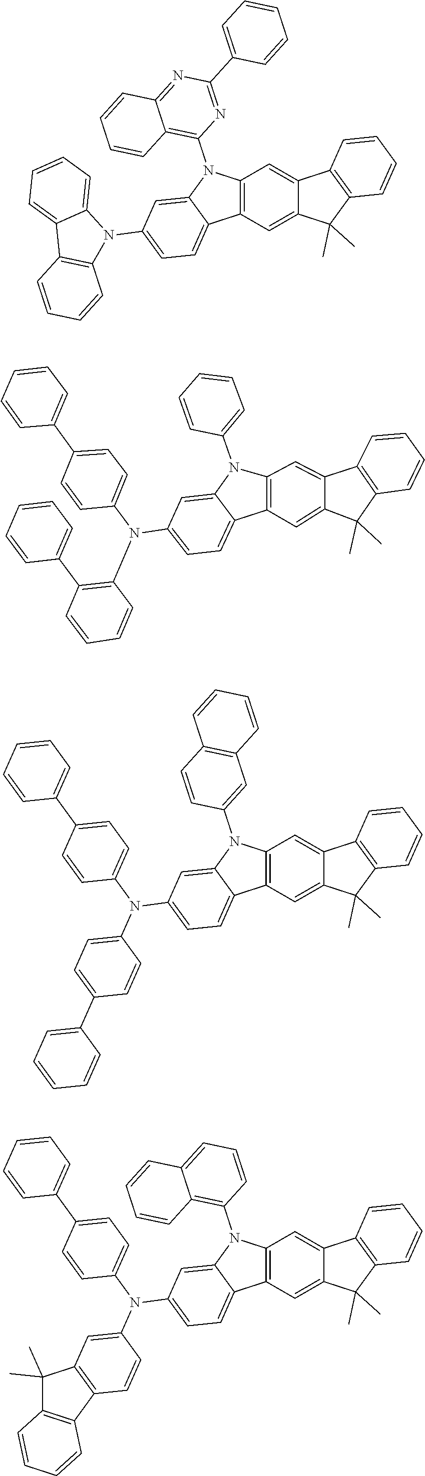 [SMILES: CC1(C)c2ccccc2-c2cc3c(cc21)c1ccc(-n2c4ccccc4c4ccccc42)cc1n3-c1nc(-c2ccccc2)nc2ccccc12 ; CC1(C)c2ccccc2-c2cc3c(cc21)c1ccc(N(c2ccc(-c4ccccc4)cc2)c2ccccc2-c2ccccc2)cc1n3-c1ccccc1 ; CC1(C)c2ccccc2-c2cc3c(cc21)c1ccc(N(c2ccc(-c4ccccc4)cc2)c2ccc(-c4ccccc4)cc2)cc1n3-c1ccc2ccccc2c1 ; CC1(C)c2ccccc2-c2ccc(N(c3ccc(-c4ccccc4)cc3)c3ccc4c5cc6c(cc5n(-c5cccc7ccccc57)c4c3)-c3ccccc3C6(C)C)cc21]

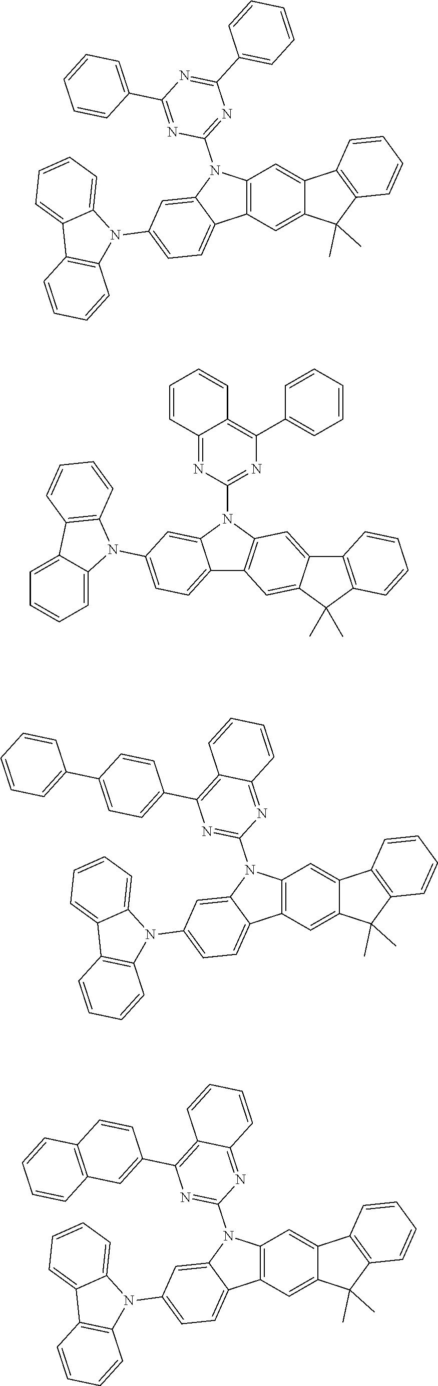 [SMILES: CC1(C)c2ccccc2-c2cc3c(cc21)c1ccc(-n2c4ccccc4c4ccccc42)cc1n3-c1nc(-c2ccccc2)nc(-c2ccccc2)n1 ; CC1(C)c2ccccc2-c2cc3c(cc21)c1ccc(-n2c4ccccc4c4ccccc42)cc1n3-c1nc(-c2ccccc2)c2ccccc2n1 ; CC1(C)c2ccccc2-c2cc3c(cc21)c1ccc(-n2c4ccccc4c4ccccc42)cc1n3-c1nc(-c2ccc(-c3ccccc3)cc2)c2ccccc2n1 ; CC1(C)c2ccccc2-c2cc3c(cc21)c1ccc(-n2c4ccccc4c4ccccc42)cc1n3-c1nc(-c2ccc3ccccc3c2)c2ccccc2n1]

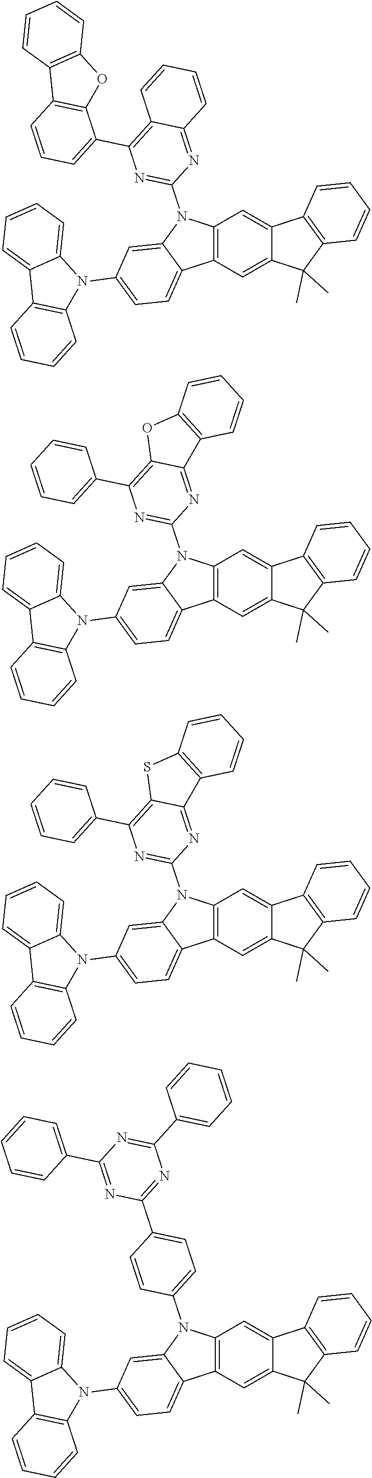 [SMILES: CC1(C)c2ccccc2-c2cc3c(cc21)c1ccc(-n2c4ccccc4c4ccccc42)cc1n3-c1nc(-c2cccc3c2oc2ccccc23)c2ccccc2n1 ; CC1(C)c2ccccc2-c2cc3c(cc21)c1ccc(-n2c4ccccc4c4ccccc42)cc1n3-c1nc(-c2ccccc2)c2oc3ccccc3c2n1 ; CC1(C)c2ccccc2-c2cc3c(cc21)c1ccc(-n2c4ccccc4c4ccccc42)cc1n3-c1nc(-c2ccccc2)c2sc3ccccc3c2n1 ; CC1(C)c2ccccc2-c2cc3c(cc21)c1ccc(-n2c4ccccc4c4ccccc42)cc1n3-c1ccc(-c2nc(-c3ccccc3)nc(-c3ccccc3)n2)cc1]

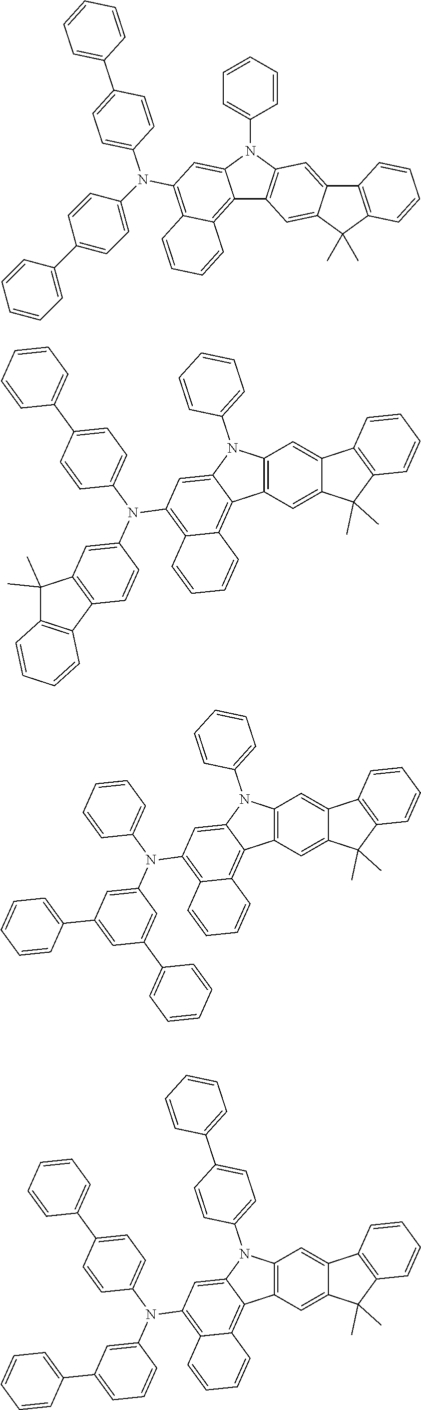

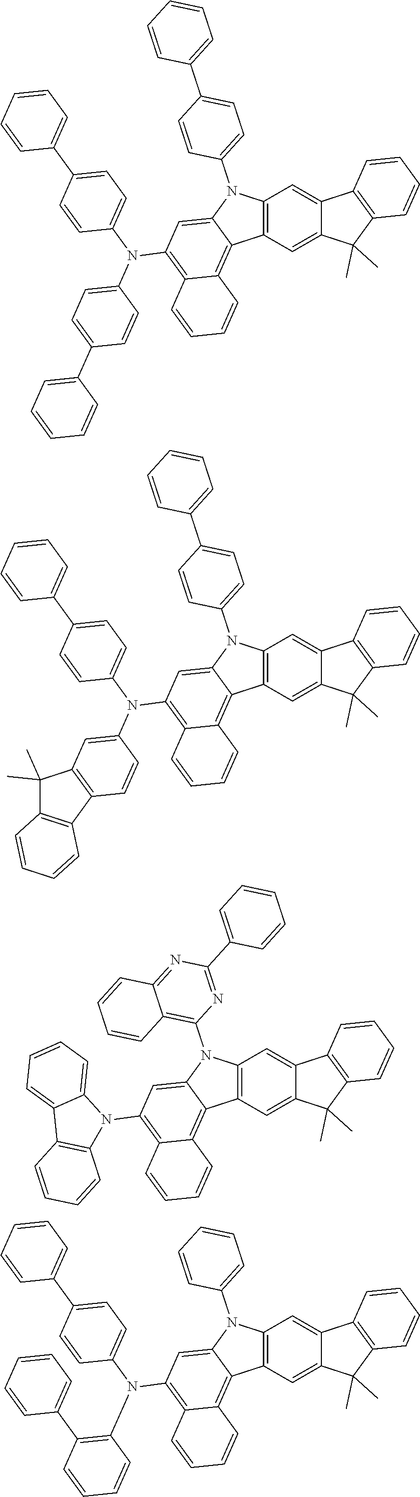

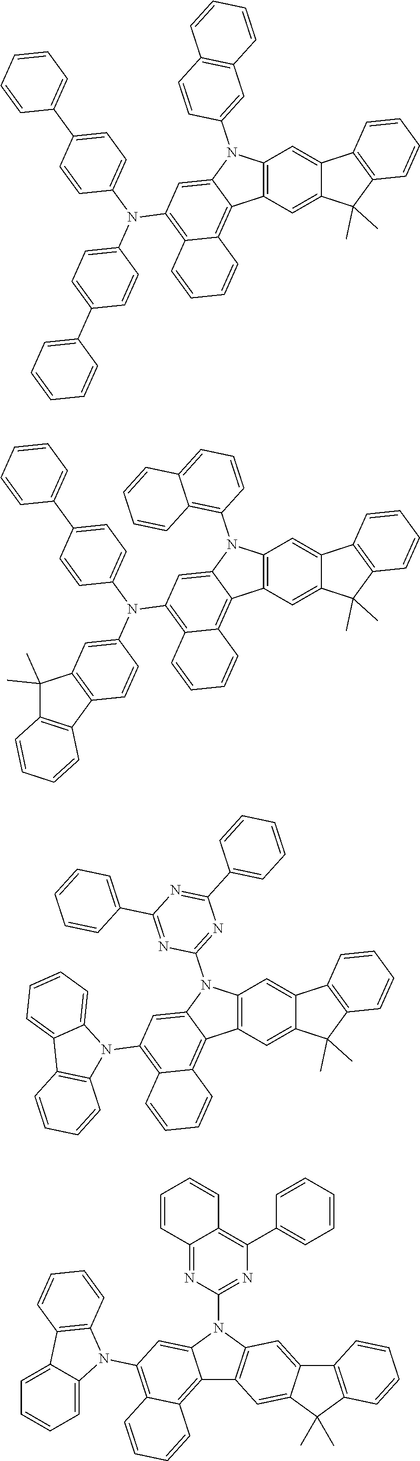

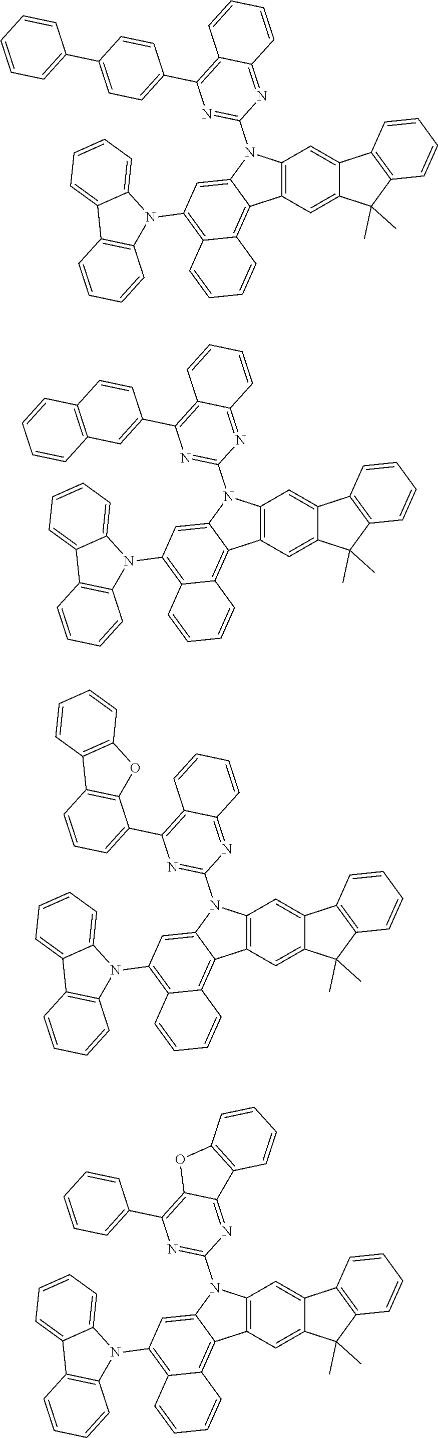

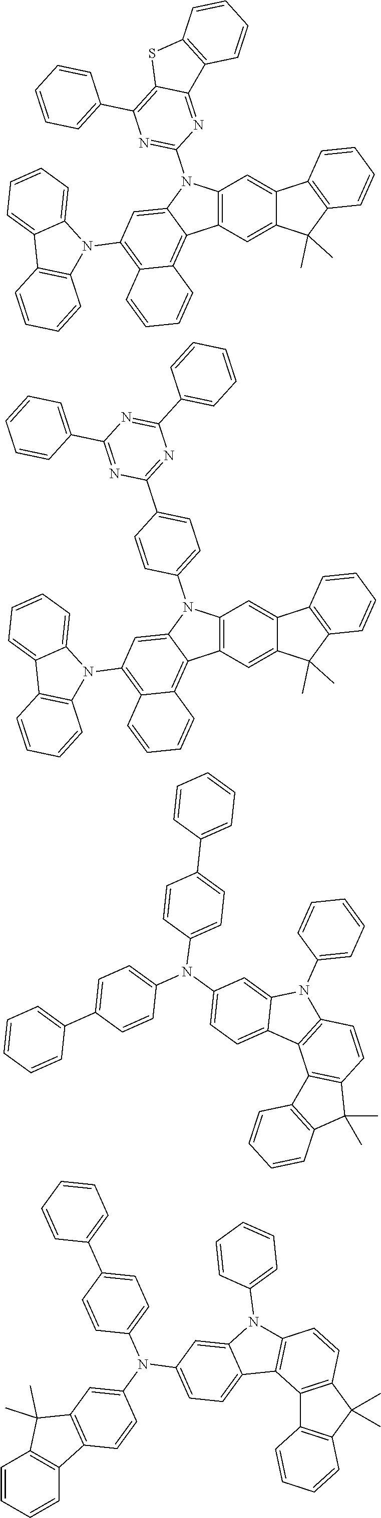

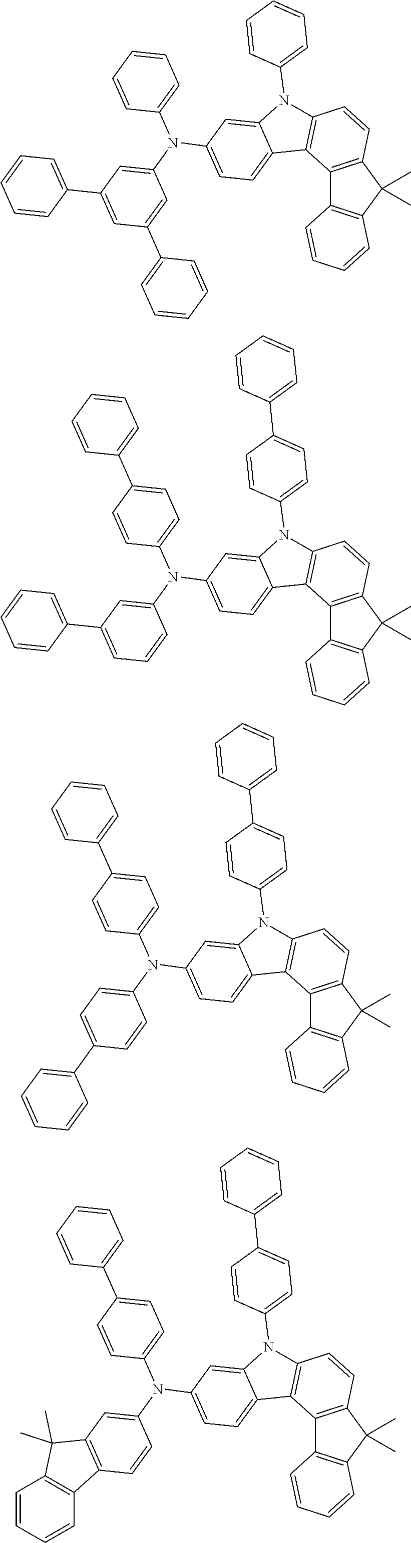 [SMILES: CC1(C)c2ccccc2-c2c1ccc1c2c2ccc(N(c3ccccc3)c3cc(-c4ccccc4)cc(-c4ccccc4)c3)cc2n1-c1ccccc1 ; CC1(C)c2ccccc2-c2c1ccc1c2c2ccc(N(c3ccc(-c4ccccc4)cc3)c3cccc(-c4ccccc4)c3)cc2n1-c1ccc(-c2ccccc2)cc1 ; CC1(C)c2ccccc2-c2c1ccc1c2c2ccc(N(c3ccc(-c4ccccc4)cc3)c3ccc(-c4ccccc4)cc3)cc2n1-c1ccc(-c2ccccc2)cc1 ; CC1(C)c2ccccc2-c2ccc(N(c3ccc(-c4ccccc4)cc3)c3ccc4c5c6c(ccc5n(-c5ccc(-c7ccccc7)cc5)c4c3)C(C)(C)c3ccccc3-6)cc21]

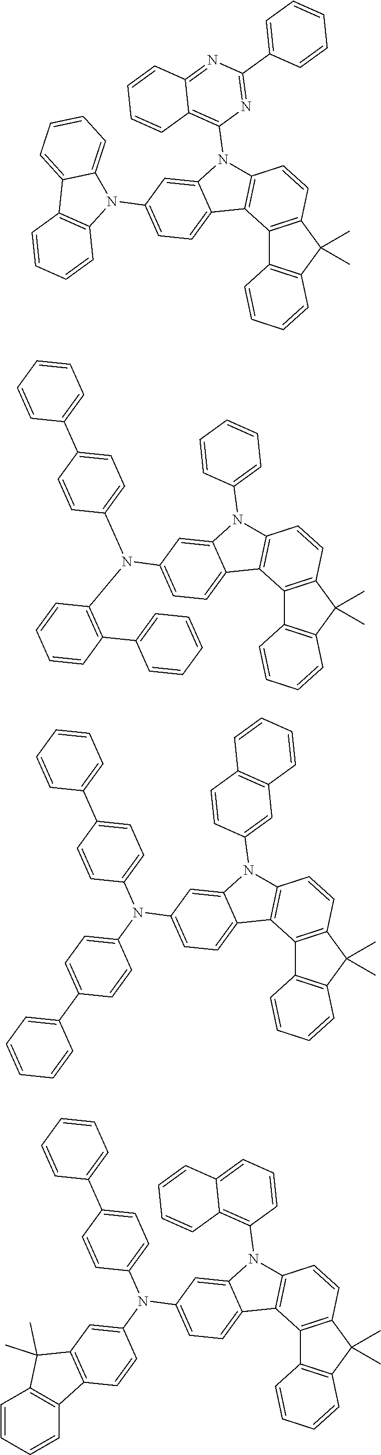

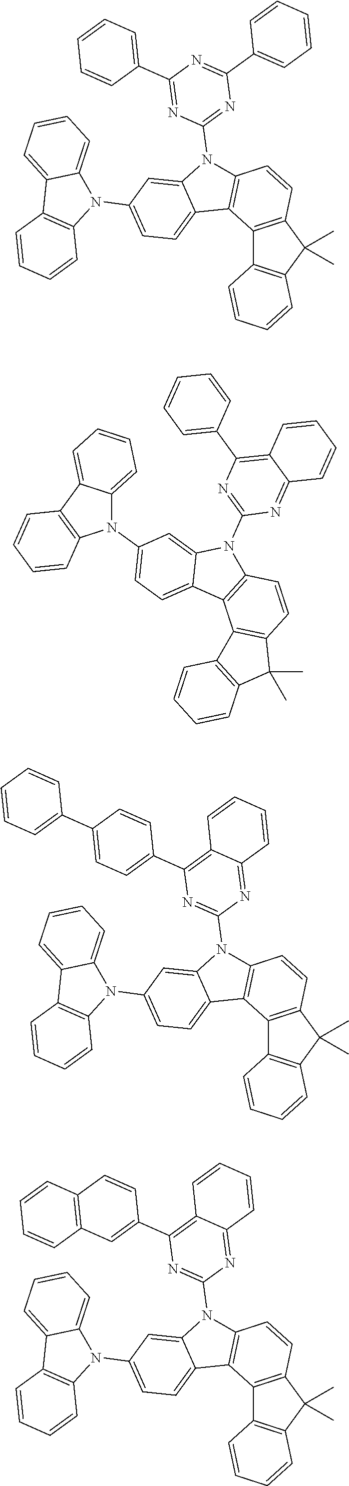 [SMILES: CC1(C)c2ccccc2-c2c1ccc1c2c2ccc(-n3c4ccccc4c4ccccc43)cc2n1-c1nc(-c2ccccc2)nc(-c2ccccc2)n1 ; CC1(C)c2ccccc2-c2c1ccc1c2c2ccc(-n3c4ccccc4c4ccccc43)cc2n1-c1nc(-c2ccccc2)c2ccccc2n1 ; CC1(C)c2ccccc2-c2c1ccc1c2c2ccc(-n3c4ccccc4c4ccccc43)cc2n1-c1nc(-c2ccc(-c3ccccc3)cc2)c2ccccc2n1 ; CC1(C)c2ccccc2-c2c1ccc1c2c2ccc(-n3c4ccccc4c4ccccc43)cc2n1-c1nc(-c2ccc3ccccc3c2)c2ccccc2n1]

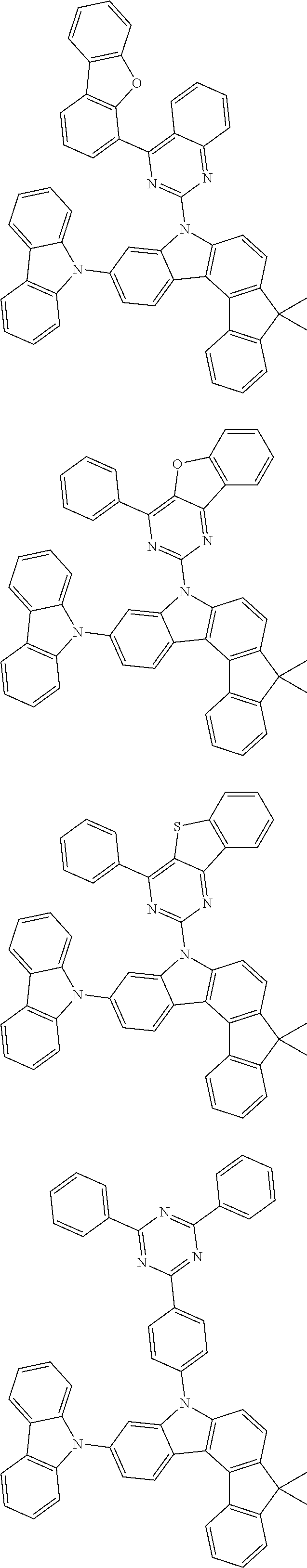

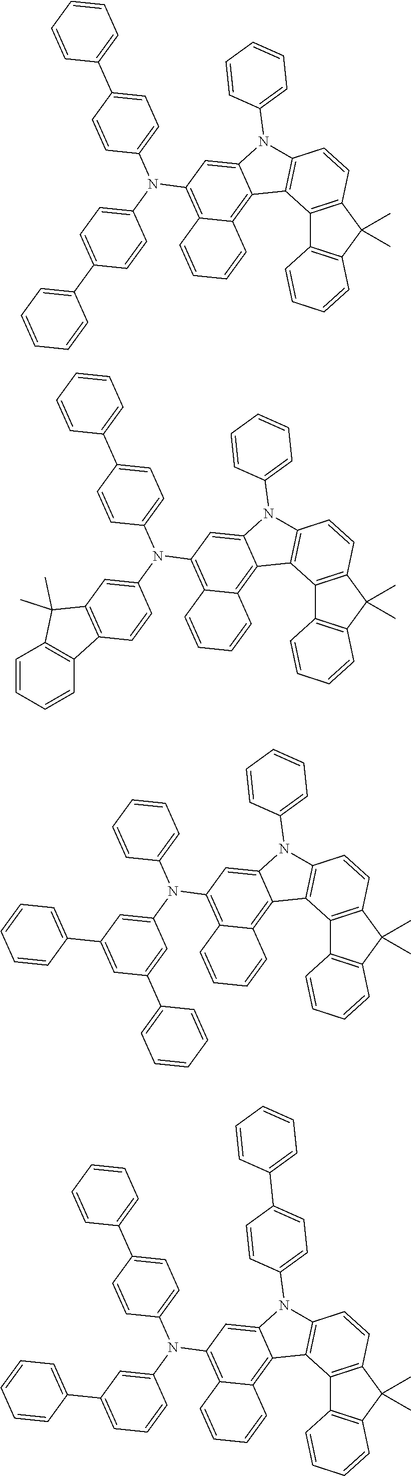

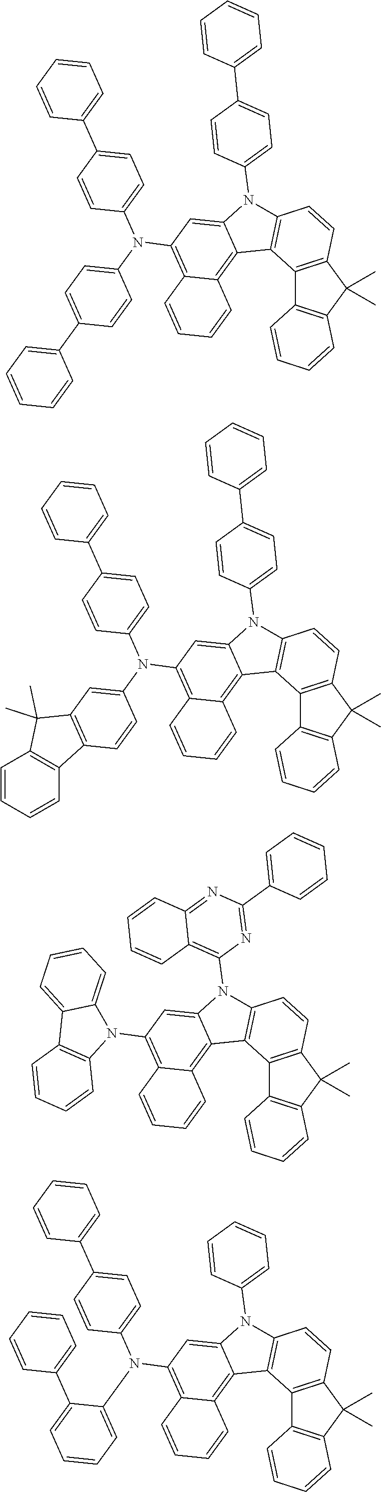

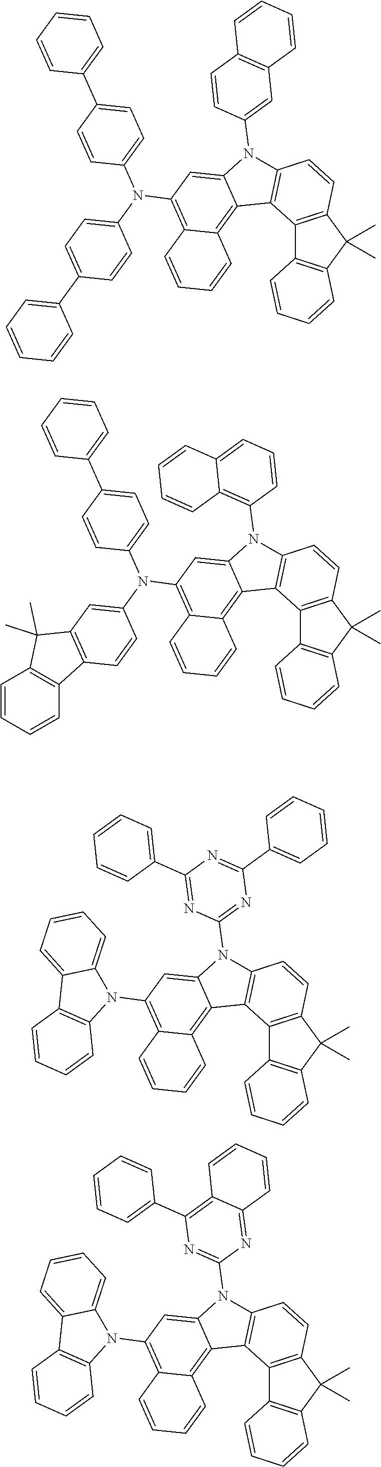

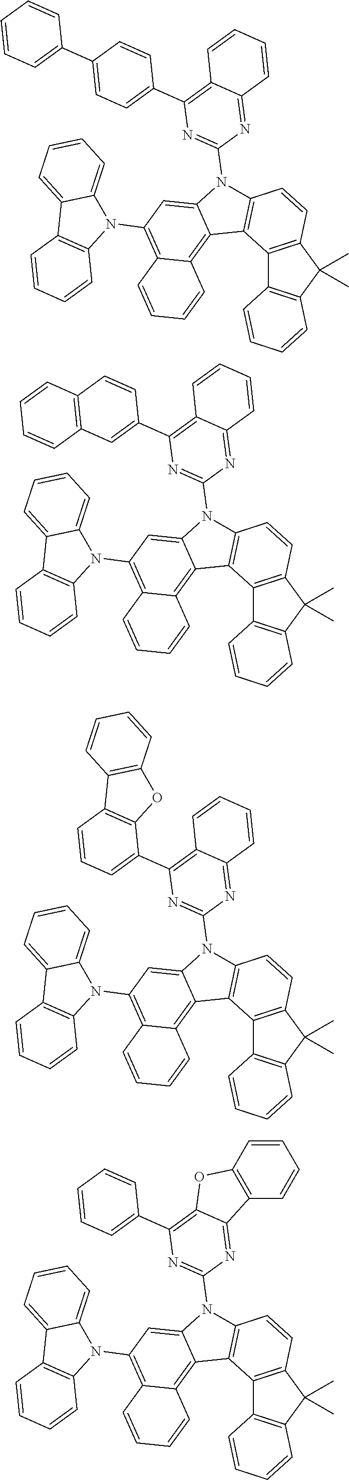

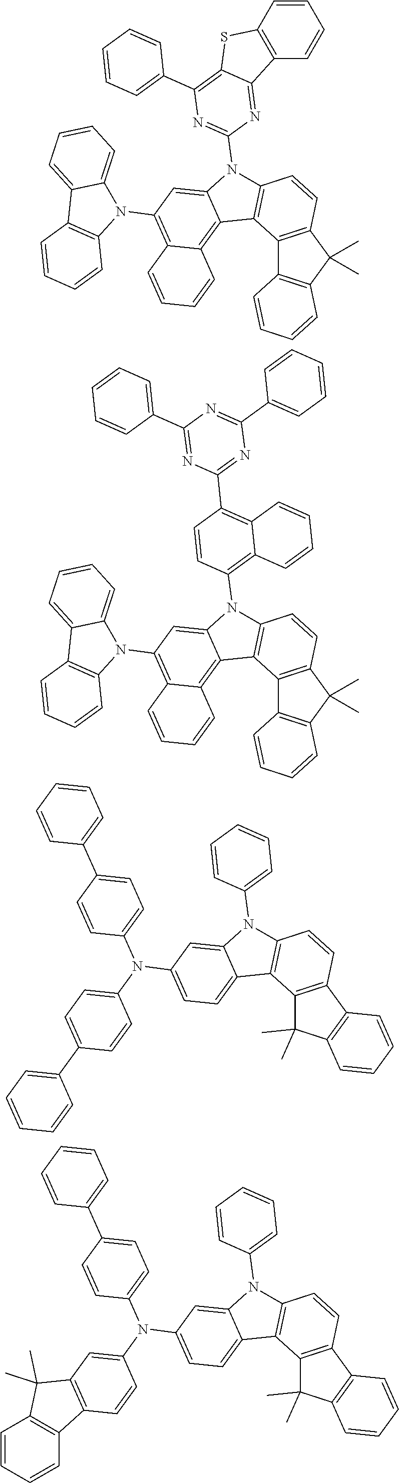

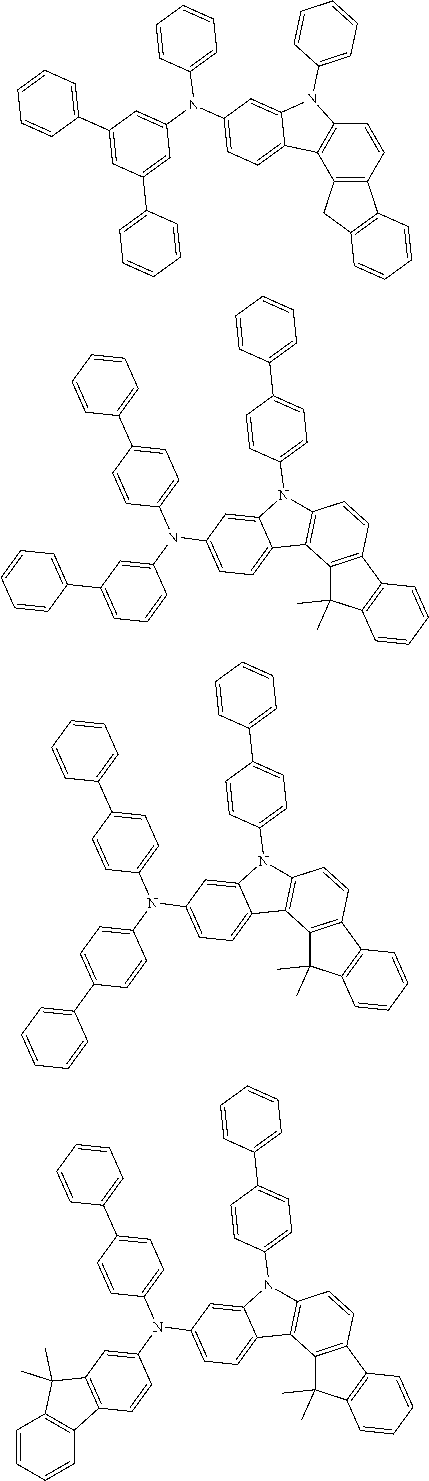 [SMILES: c1ccc(-c2cc(-c3ccccc3)cc(N(c3ccccc3)c3ccc4c5c6c(ccc5n(-c5ccccc5)c4c3)-c3ccccc3C6)c2)cc1 ; CC1(C)c2ccccc2-c2ccc3c(c21)c1ccc(N(c2ccc(-c4ccccc4)cc2)c2cccc(-c4ccccc4)c2)cc1n3-c1ccc(-c2ccccc2)cc1 ; CC1(C)c2ccccc2-c2ccc3c(c21)c1ccc(N(c2ccc(-c4ccccc4)cc2)c2ccc(-c4ccccc4)cc2)cc1n3-c1ccc(-c2ccccc2)cc1 ; CC1(C)c2ccccc2-c2ccc(N(c3ccc(-c4ccccc4)cc3)c3ccc4c5c6c(ccc5n(-c5ccc(-c7ccccc7)cc5)c4c3)-c3ccccc3C6(C)C)cc21]

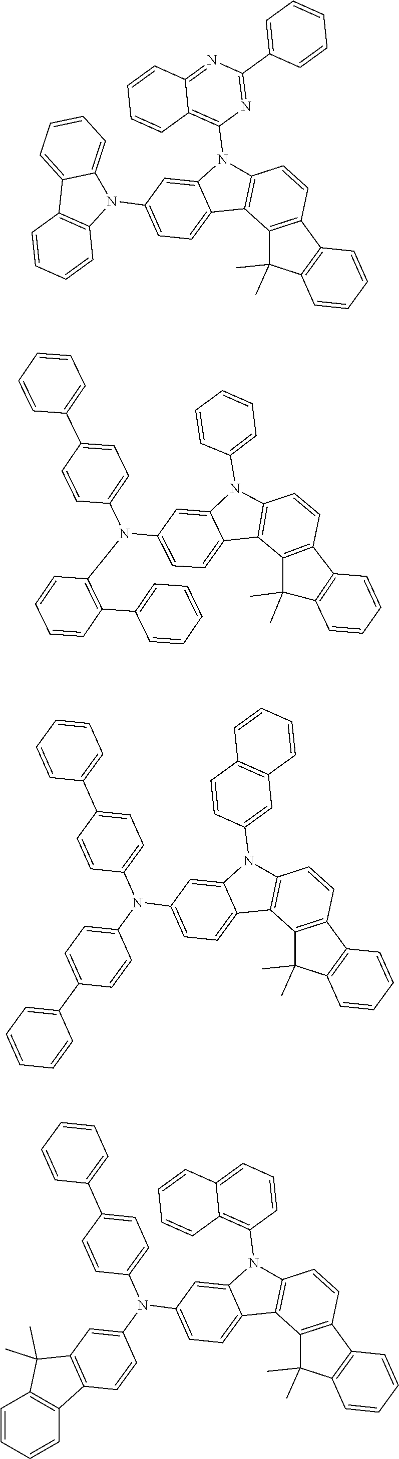 [SMILES: CC1(C)c2ccccc2-c2ccc3c(c21)c1ccc(-n2c4ccccc4c4ccccc42)cc1n3-c1nc(-c2ccccc2)nc2ccccc12 ; CC1(C)c2ccccc2-c2ccc3c(c21)c1ccc(N(c2ccc(-c4ccccc4)cc2)c2ccccc2-c2ccccc2)cc1n3-c1ccccc1 ; CC1(C)c2ccccc2-c2ccc3c(c21)c1ccc(N(c2ccc(-c4ccccc4)cc2)c2ccc(-c4ccccc4)cc2)cc1n3-c1ccc2ccccc2c1 ; CC1(C)c2ccccc2-c2ccc(N(c3ccc(-c4ccccc4)cc3)c3ccc4c5c6c(ccc5n(-c5cccc7ccccc57)c4c3)-c3ccccc3C6(C)C)cc21]

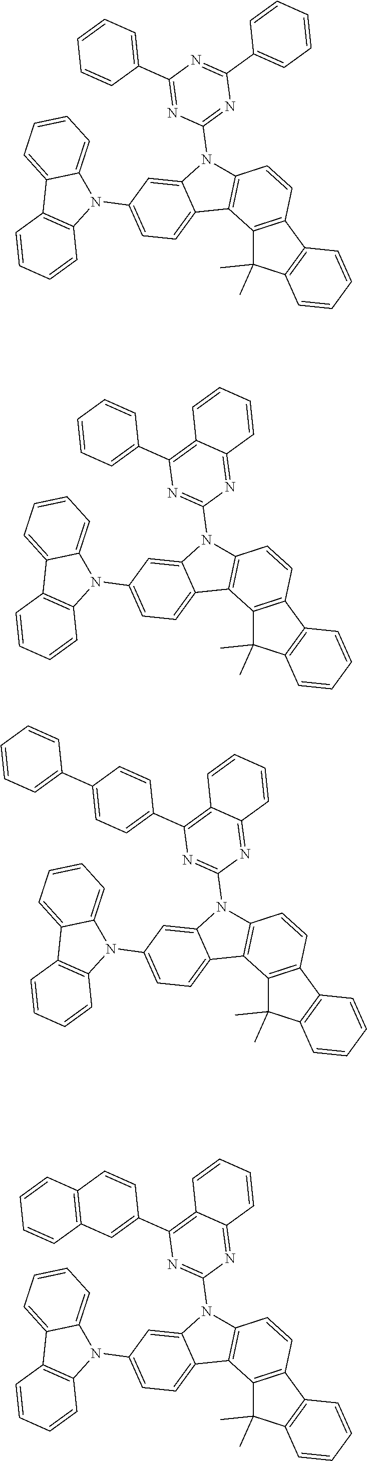 [SMILES: CC1(C)c2ccccc2-c2ccc3c(c21)c1ccc(-n2c4ccccc4c4ccccc42)cc1n3-c1nc(-c2ccccc2)nc(-c2ccccc2)n1 ; CC1(C)c2ccccc2-c2ccc3c(c21)c1ccc(-n2c4ccccc4c4ccccc42)cc1n3-c1nc(-c2ccccc2)c2ccccc2n1 ; CC1(C)c2ccccc2-c2ccc3c(c21)c1ccc(-n2c4ccccc4c4ccccc42)cc1n3-c1nc(-c2ccc(-c3ccccc3)cc2)c2ccccc2n1 ; CC1(C)c2ccccc2-c2ccc3c(c21)c1ccc(-n2c4ccccc4c4ccccc42)cc1n3-c1nc(-c2ccc3ccccc3c2)c2ccccc2n1]

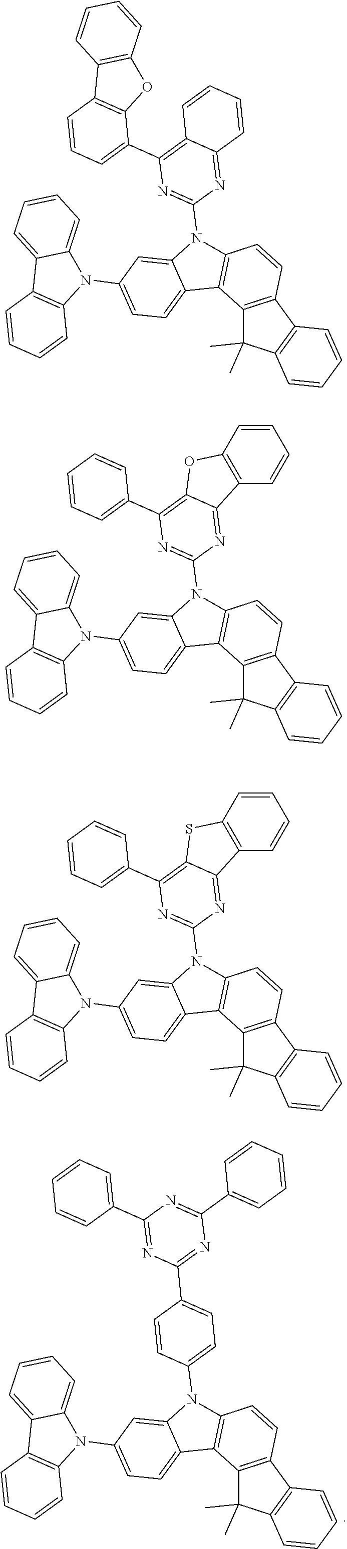

The compound represented by Chemical Formula 1 can be prepared by a method as shown in the following Reaction Scheme 1.

The above preparation method can be further specified in preparation examples to be described later.

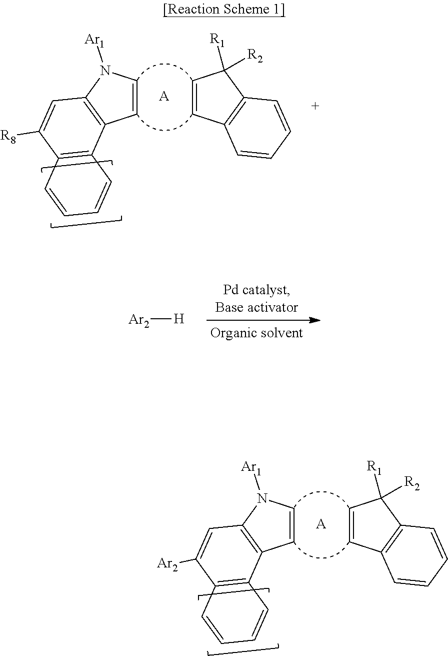

In Reaction Scheme 1, A, R₁, R₂, Ar₁, Ar₂, and n are as defined in Chemical Formula 1, and R₈ is a halogen group such as fluoro, chloro, bromo, iodo, and the like.

Specifically, the above reaction utilizes a Buchwald-Hartwig reaction, and can be carried out in the presence of a palladium-based catalyst (Pd catalyst) compound such as Pd(P-tBu₃)₂ or the like.

Further, the reaction can be carried out together with one or more base activators such as NaOtBu, K₂CO₃, Cs₂CO₃, or the like in the presence of one or more organic solvents such as dichloromethane, ethyl acetate, diethyl ether, acetonitrile, isopropyl alcohol, acetone, tetrahydrofuran (THF), N,N-dimethylformamide (DMF), dimethyl sulfoxide (DMSO), toluene, or xylene.

In still another embodiment of the invention, an organic light emitting device is provided, including a compound represented by Chemical Formula 1. As an example, an organic light emitting device is provided, including: a first electrode; a second electrode provided opposite to the first electrode; and one or more organic material layers provided between the first electrode and the second electrode, wherein one or more layers of the organic material layers include the compound represented by Chemical Formula 1.

The organic material layer of the organic light emitting device of the present invention can have a single layer structure, or it can have a multilayered structure in which two or more organic material layers are stacked. For example, the organic light emitting device of the present disclosure can have a structure including a hole injection layer, a hole transport layer, a light emitting layer, an electron transport layer, an electron injection layer, and the like as the organic material layer. However, the structure of the organic light emitting device is not limited thereto, and it can include a smaller number of organic layers.

Further, the organic material layer can include a hole injection layer, a hole transport layer, or a layer simultaneously performing hole injection and hole transport, wherein the hole injection layer, the hole transport layer, or the layer simultaneously performing hole injection and hole transport include a compound represented by Chemical Formula 1.

Further, the organic material layer can include a light emitting layer, wherein the light emitting layer includes a compound represented by Chemical Formula 1.

Further, the organic material layer can include an electron transport layer or an electron injection layer, wherein the electron transport layer or the electron injection layer includes a compound represented by Chemical Formula 1.

Further, the electron transport layer, the electron injection layer, or a layer simultaneously performing electron transport and electron injection include a compound represented by Chemical Formula 1.

Further, the organic material layer includes a light emitting layer and an electron transport layer, wherein the electron transport layer can include a compound represented by Chemical Formula 1.

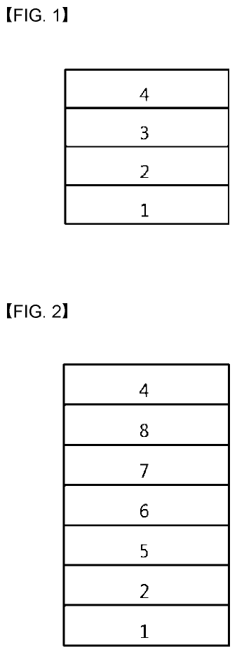

Further, the organic light emitting device according to the present invention can be a normal type of organic light emitting device in which an anode (positive electrode), one or more organic material layers, and a cathode (negative electrode) are sequentially stacked on a substrate. Further, the organic light emitting device according to the present disclosure can be an inverted type of organic light emitting device in which a cathode, one or more organic material layers, and an anode are sequentially stacked on a substrate. For example, the structure of an organic light emitting device according to an embodiment of the present disclosure is illustrated in FIGS. 1 and 2.

FIG. 1 shows an example of an organic light emitting device including a substrate 1, an anode 2, a light emitting layer 3, and a cathode 4. In such a structure, the compound represented by Chemical Formula 1 can be included in the light emitting layer.

FIG. 2 shows an example of an organic light emitting device including a substrate 1, an anode 2, a hole injection layer 5, a hole transport layer 6, a light emitting layer 7, an electron transport layer 8, and a cathode 4. In such a structure, the compound represented by Chemical Formula 1 can be included in one or more layers of the hole injection layer, the hole transport layer, the light emitting layer, and the electron transport layer.

The organic light emitting device according to the present invention can be manufactured by materials and methods known in the art, except that one or more layers of the organic material layers includes the compound represented by Chemical Formula 1. In addition, when the organic light emitting device includes a plurality of organic material layers, the organic material layers can be formed of the same material or different materials.

For example, the organic light emitting device according to the present invention can be manufactured by sequentially stacking a first electrode, an organic material layer, and a second electrode on a substrate. In this case, the organic light emitting device can be manufactured by depositing a metal, metal oxides having conductivity, or an alloy thereof on the substrate using a PVD (physical vapor deposition) method such as a sputtering method or an e-beam evaporation method to form an anode, forming organic material layers including the hole injection layer, the hole transport layer, the light emitting layer, and the electron transport layer thereon, and then depositing a material that can be used as the cathode thereon. In addition to such a method, the organic light emitting device can be manufactured by sequentially depositing a cathode material, an organic material layer, and an anode material on a substrate.

In addition, the compound represented by Chemical Formula 1 can be formed into an organic layer by a solution coating method as well as a vacuum deposition method at the time of manufacturing an organic light emitting device. Herein, the solution coating method means spin coating, dip coating, doctor blading, inkjet printing, screen printing, spraying, roll coating, or the like, but is not limited thereto.

In addition to such a method, the organic light emitting device can be manufactured by sequentially depositing a cathode material, an organic material layer, and an anode material on a substrate (International Publication WO2003/012890). However, the manufacturing method is not limited thereto.

As an example, the first electrode is an anode and the second electrode is a cathode, or alternatively the first electrode is a cathode and the second electrode is an anode.

As the anode material, generally, a material having a large work function is preferably used so that holes can be smoothly injected into the organic material layer. Specific examples of the anode material include metals such as vanadium, chrome, copper, zinc, and gold, or alloys thereof; metal oxides such as zinc oxides, indium oxides, indium tin oxides (ITO), and indium zinc oxides (IZO); a combination of metals and oxides, such as ZnO:Al or SNO₂:Sb; conductive polymers such as poly(3-methylthiophene), poly[3,4-(ethylene-1,2-dioxy)thiophene] (PEDOT), polypyrrole, and polyaniline, and the like, but are not limited thereto.

As the cathode material, generally, a material having a small work function is preferably used so that electrons can be easily injected into the organic material layer. Specific examples of the cathode material include metals such as magnesium, calcium, sodium, potassium, titanium, indium, yttrium, lithium, gadolinium, aluminum, silver, tin, and lead, or alloys thereof; a multilayered structure material such as LiF/Al or LiO₂/Al; and the like, but are not limited thereto.

The hole injection layer is a layer for injecting holes from the electrode, and the hole injection material is preferably a compound which has a capability of transporting the holes, thus has a hole injecting effect in the anode and an excellent hole injecting effect to the light emitting layer or the light emitting material, prevents excitons produced in the light emitting layer from moving to a hole injection layer or the electron injection material, and is excellent in the ability to form a thin film. It is preferable that a HOMO (highest occupied molecular orbital) of the hole injection material is between the work function of the anode material and a HOMO of a peripheral organic material layer. Specific examples of the hole injection material include metal porphyrin, oligothiophene, an arylamine-based organic material, a hexanitrilehexaazatriphenylene-based organic material, a quinacridone-based organic material, a perylene-based organic material, anthraquinone, a polyaniline and polythiophene-based conductive polymer, and the like, but are not limited thereto.

The hole transport layer is a layer that receives holes from a hole injection layer and transports the holes to the light emitting layer, and is suitably a material having large mobility to the holes, which can receive holes from the anode or the hole injection layer and transfer the holes to the light emitting layer. Specific examples thereof include an arylamine-based organic material, a conductive polymer, a block copolymer in which a conjugate portion and a non-conjugate portion are present together, and the like, but are not limited thereto.

The light emitting material is preferably a material which can receive holes and electrons transported from a hole transport layer and an electron transport layer, respectively, and combine the holes and the electrons to emit light in a visible ray region, and has good quantum efficiency to fluorescence or phosphorescence. Specific examples of the light emitting material include: an 8-hydroxy-quinoline aluminum complex (Alq₃); a carbazole-based compound; a dimerized styryl compound; BAlq; a 10-hydroxybenzoquinoline-metal compound; a benzoxazole, benzothiazole, and benzimidazole-based compound; a poly(p-phenylene vinylene) (PPV)-based polymer; a spiro compound; polyfluorene, rubrene; and the like, but are not limited thereto.

The light emitting layer can include a host material and a dopant material. The host material can be a fused aromatic ring derivative, a heterocycle-containing compound, or the like. Specific examples of the fused aromatic ring derivatives include anthracene derivatives, pyrene derivatives, naphthalene derivatives, pentacene derivatives, phenanthrene compounds, fluoranthene compounds, and the like. Examples of the heterocyclic-containing compounds include carbazole derivatives, dibenzofuran derivatives, ladder-type furan compounds, pyrimidine derivatives, and the like, but are not limited thereto.

Examples of the dopant material include an aromatic amine derivative, a styrylamine compound, a boron complex, a fluoranthene compound, a metal complex, and the like. Specifically, the aromatic amine derivative is a substituted or unsubstituted fused aromatic ring derivative having an arylamino group, and examples thereof include pyrene, anthracene, chrysene, periflanthene, and the like, which have an arylamino group. The styrylamine compound is a compound where at least one arylvinyl group is substituted in a substituted or unsubstituted arylamine, in which one or two or more substituent groups selected from the group consisting of an aryl group, a silyl group, an alkyl group, a cycloalkyl group, and an arylamino group are substituted or unsubstituted. Specific examples thereof include styrylamine, styryldiamine, styryltriamine, styryltetramine, and the like, but are not limited thereto. Further, the metal complex includes an iridium complex, a platinum complex, and the like, but is not limited thereto.

The electron transport layer is a layer which receives electrons from an electron injection layer and transports the electrons to a light emitting layer, and an electron transport material is suitably a material which can receive electrons well from a cathode and transfer the electrons to a light emitting layer, and has large mobility for electrons. Specific examples of the electron transport material include: an Al complex of 8-hydroxyquinoline; a complex including Alq₃; an organic radical compound; a hydroxyflavone-metal complex; and the like, but are not limited thereto. The electron transport layer can be used with any desired cathode material, as used according to the related art. In particular, appropriate examples of the cathode material are typical materials which have a low work function, followed by an aluminum layer or a silver layer. Specific examples thereof include cesium, barium, calcium, ytterbium, and samarium, in each case followed by an aluminum layer or a silver layer.

The electron injection layer is a layer which injects electrons from an electrode, and is preferably a compound which has a capability of transporting electrons, has an effect of injecting electrons from a cathode, and an excellent effect of injecting electrons into a light emitting layer or a light emitting material, prevents excitons produced from the light emitting layer from moving to a hole injection layer, and is also excellent in the ability to form a thin film. Specific examples of the electron injection layer include fluorenone, anthraquinodimethane, diphenoquinone, thiopyran dioxide, oxazole, oxadiazole, triazole, imidazole, perylenetetracarboxylic acid, fluorenylidene methane, anthrone, and the like, and derivatives thereof, a metal complex compound, a nitrogen-containing 5-membered ring derivative, and the like, but are not limited thereto.

Examples of the metal complex compound include 8-hydroxyquinolinato lithium, bis(8-hydroxyquinolinato)zinc, bis(8-hydroxyquinolinato)copper, bis(8-hydroxyquinolinato)manganese, tris(8-hydroxyquinolinato)aluminum, tris(2-methyl-8-hydroxyquinolinato)aluminum, tris(8-hydroxyquinolinato)gallium, bis(10-hydroxybenzo[h]quinolinato)beryllium, bis(10-hydroxybenzo[h]quinolinato)zinc, bis(2-methyl-8-quinolinato)chlorogallium, bis(2-methyl-8-quinolinato)(o-cresolato)gallium, bis(2-methyl-8-quinolinato)(1-naphtholato)aluminum, bis(2-methyl-8-quinolinato)(2-naphtholato)gallium, and the like, but are not limited thereto.

The organic light emitting device according to the present invention can be a front side emission type, a back side emission type, or a double side emission type according to the used material.

In addition, the compound represented by Chemical Formula 1 can be included in an organic solar cell or an organic transistor in addition to an organic light emitting device.

The preparation of the compound represented by Chemical Formula 1 and the organic light emitting device including the same will be described in detail in the following examples. However, these examples are presented for illustrative purposes only, and are not intended to limit the scope of the present invention.

### SYNTHESIS EXAMPLES

### Synthesis Example 1

### 1-1) Synthesis of Compound A-1

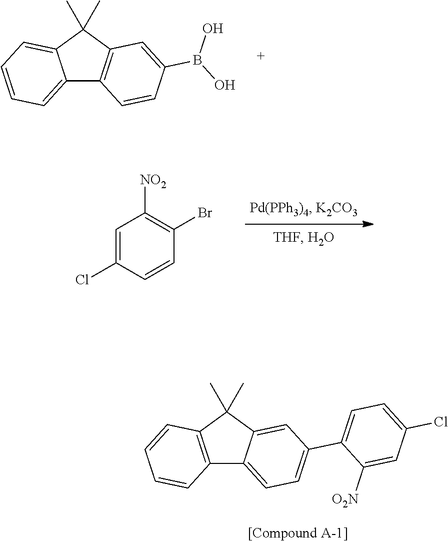 [SMILES: CC1(C)c2ccccc2-c2ccc(B(O)O)cc21 ; O=[N+]([O-])c1cc(Cl)ccc1Br ; CC1(C)c2ccccc2-c2ccc(-c3ccc(Cl)cc3[N+](=O)[O-])cc21]

In a three-necked flask, (9,9-dimethyl-9H-fluoren-2-yl)boronic acid (30.0 g, 126.0 mmol) and 1-bromo-4-chloro-2-nitrobenzene (31.3 g, 132.3 mmol) were dissolved in 450 mL of THF, and K₂CO₃(69.7 g, 504.0 mmol) was dissolved in 150 mL of H₂O and added. Pd(PPh₃)₄ (7.3 g, 6.3 mmol) was added thereto, and the reaction mixture was stirred for 8 hours under an argon atmosphere. After the reaction was completed, the reaction solution was cooled to room temperature, then transferred to a separatory funnel, and extracted with ethyl acetate. The extract was dried over MgSO₄, filtered, and concentrated, and then recrystallized with EtOH to obtain 37.5 g of Compound A-1. (Yield: 85%, MS [M+H]⁺=350)

### 1-2) Synthesis of Compound A and Compound B

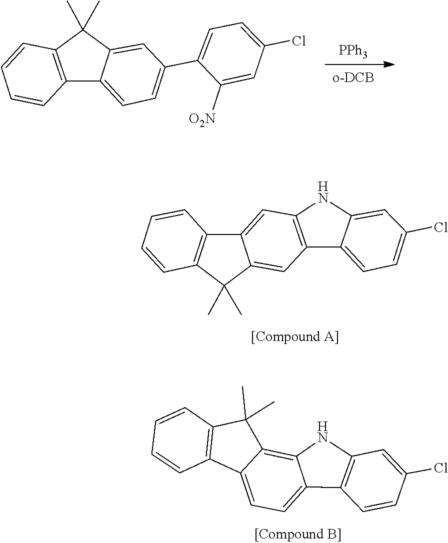 [SMILES: CC1(C)c2ccccc2-c2ccc(-c3ccc(Cl)cc3[N+](=O)[O-])cc21 ; "o-DCB" ; CC1(C)c2ccccc2-c2cc3[nH]c4cc(Cl)ccc4c3cc21 ; CC1(C)c2ccccc2-c2ccc3c([nH]c4cc(Cl)ccc43)c21 ; "PPh3"]

Compound A-1 (35.0 g, 100.1 mmol), triphenylphosphine (20.7 g, 150.1 mmol), and 350 mL of o-dichlorobenzene were added to a two-necked flask, and the mixture was stirred under reflux conditions for 24 hours. After the reaction was completed, the reaction solution was cooled to room temperature, and the solvent was removed by distillation under reduced pressure and extracted with CH₂Cl₂. The extract was dried over MgSO₄, filtered, and concentrated. The sample was purified by silica gel column chromatography to obtain 16.2 g (yield: 51%) of Compound A and 11.1 g (yield: 35%) of Compound B. (MS [M+H]⁺=318)

### Synthesis Example 2

### 2-1) Synthesis of Compound C-1

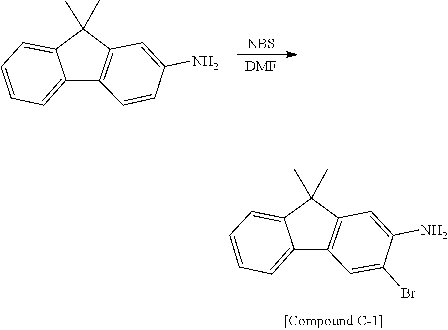

In a two-necked flask, 9,9-dimethyl-9H-fluoren-2-amine (20.0 g, 95.6 mmol) was dissolved in 400 mL of DMF, N-bromosuccinimide (NBS, 17.0 g, 95.6 mmol) was slowly added thereto, and the mixture was stirred at room temperature for 8 hours. After the reaction was completed, the reaction solution was transferred to a separatory funnel, and water (300 mL) was added thereto, followed by extraction with ethyl acetate. The sample was purified by silica gel column chromatography to obtain 22.6 g of Compound C-1. (Yield: 82%, MS [M+H]⁺=288)

### 2-2) Synthesis of Compound C-2

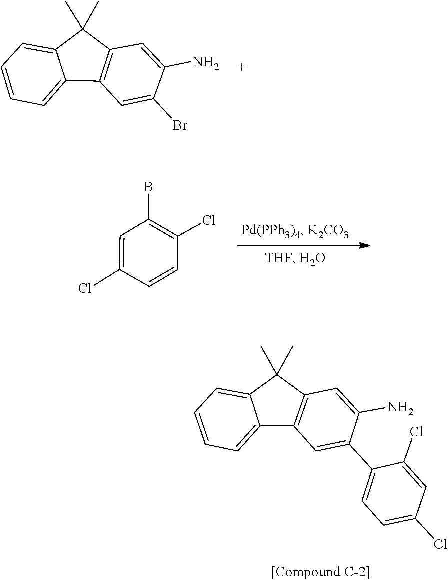

In a three-necked flask, Compound C-1 (20.0 g, 69.4 mmol) and (2,5-dichlorophenyl)boronic acid (14.6 g, 76.3 mmol) were dissolved in 300 mL of THF, and K₂CO₃ (38.4 g, 277.6 mmol) was dissolved in 150 mL of H₂O and added. Pd(PPh₃)₄ (4.0 g, 3.5 mmol) was added thereto, and the mixture was stirred for 8 hours under an argon atmosphere. After the reaction was completed, the reaction solution was cooled to room temperature, then transferred to a separatory funnel and extracted with ethyl acetate. The extract was dried over MgSO₄, filtered, and concentrated. The sample was then purified by silica gel column chromatography to obtain 19.2 g of Compound C-2. (Yield: 78%, MS [M+H]⁺=354)

### 2-3) Synthesis of Compound C

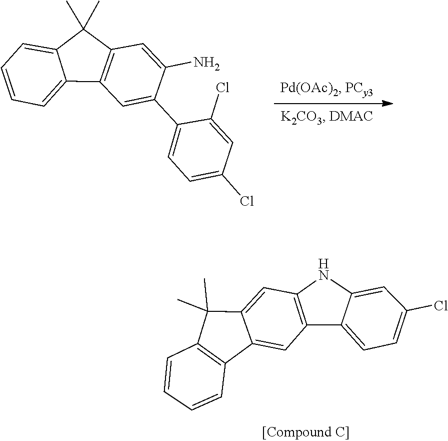 [SMILES: CC1(C)c2ccccc2-c2cc(-c3ccc(Cl)cc3Cl)c(N)cc21 ; CC1(C)c2ccccc2-c2cc3c(cc21)[nH]c1cc(Cl)ccc13]

Compound C-2 (18.0 g, 50.8 mmol), Pd(OAc)₂ (1.0 g, 4.1 mmol), tricyclohexylphosphine (PCy₃, 2.3 g, 8.1 mmol), K₂CO₃ (28.1 g, 203.2 mmol), and 540 mL of DMAC were added to a three-necked flask, and the mixture was stirred for 10 hours under an argon atmosphere. After the reaction was completed, the reaction solution was cooled to room temperature, 200 mL of H₂O was added thereto, and then transferred to a separatory funnel and extracted with ethyl acetate. The extract was dried over MgSO₄ and concentrated. The sample was then purified by silica gel column chromatography to obtain 12.1 g of Compound C. (Yield: 75%, MS [M+H]⁺=318)

### Synthesis Example 3

### 3-1) Synthesis of Compound D-1

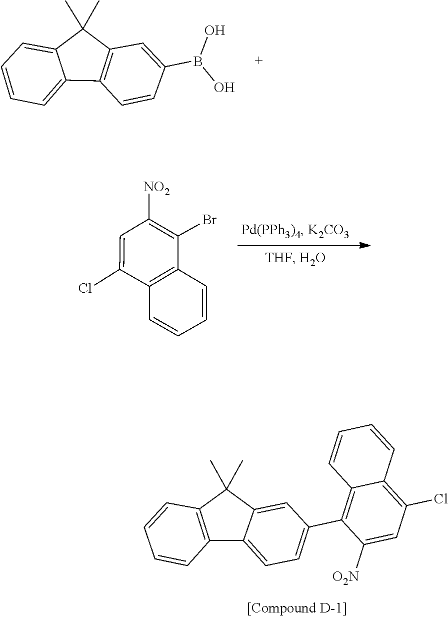 [SMILES: CC1(C)c2ccccc2-c2ccc(B(O)O)cc21 ; O=[N+]([O-])c1cc(Cl)c2ccccc2c1Br ; CC1(C)c2ccccc2-c2ccc(-c3c([N+](=O)[O-])cc(Cl)c4ccccc34)cc21]

In a three-necked flask, (9,9-dimethyl-9H-fluoren-2-yl)boronic acid (20.0 g, 84.0 mmol) and 1-bromo-4-chloro-2-nitrobenzene (25.3 g, 88.2 mmol) were dissolved in 300 mL of THF, and K₂CO₃ (46.4 g, 336.0 mmol) was dissolved in 120 mL of H₂O and added. Pd(PPh₃)₄ (4.9 g, 4.2 mmol) was added thereto, and the mixture was stirred for 8 hours under an argon atmosphere. After the reaction was completed, the reaction solution was cooled to room temperature, then transferred to a separatory funnel and extracted with ethyl acetate. The extract was dried over MgSO₄, filtered, and concentrated, and then recrystallized with EtOH to obtain 28.6 g of Compound D-1. (Yield: 85%, MS [M+H]⁺=400)

### 3-2) Synthesis of Compound D

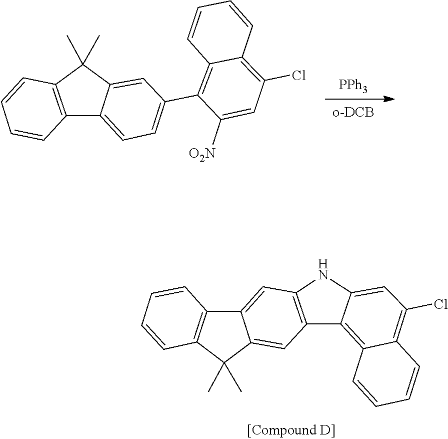 [SMILES: CC1(C)c2ccccc2-c2ccc(-c3c([N+](=O)[O-])cc(Cl)c4ccccc34)cc21 ; "o-DCB" ; CC1(C)c2ccccc2-c2cc3[nH]c4cc(Cl)c5ccccc5c4c3cc21 ; "PPh3"]

Compound D-1 (27.0 g, 67.5 mmol), triphenylphosphine (14.0 g, 101.3 mmol), and o-dichlorobenzene (270 mL) were added to a two-necked flask, and the reaction mixture was stirred under reflux conditions for 24 hours. After the reaction was completed, the reaction solution was cooled to room temperature, and the solvent was removed by distillation under reduced pressure and extracted with CH₂Cl₂. The extract was dried over MgSO₄, filtered, and concentrated. The sample was then purified by silica gel column chromatography to obtain 10.7 g of Compound D. (Yield: 43%, MS [M+H]⁺=318)

### Synthesis Example 4

### 4-1) Synthesis of Compound E-1

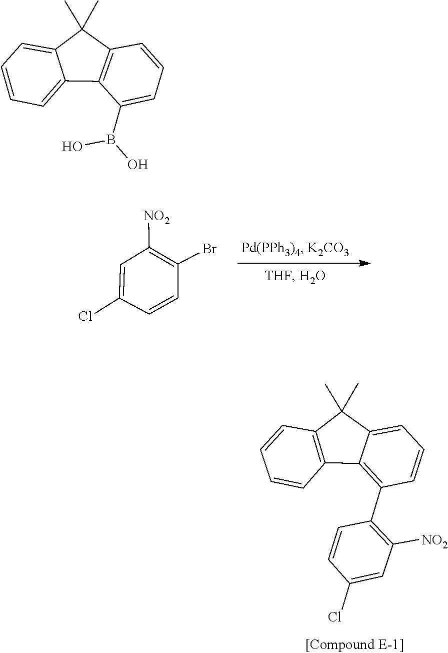 [SMILES: CC1(C)c2ccccc2-c2c(B(O)O)cccc21 ; O=[N+]([O-])c1cc(Cl)ccc1Br ; CC1(C)c2ccccc2-c2c(-c3ccc(Cl)cc3[N+](=O)[O-])cccc21]

In a three-necked flask, (9,9-dimethyl-9H-fluoren-2-yl)boronic acid (20.0 g, 84.0 mmol) and 1-bromo-4-chloro-2-nitrobenzene (20.9 g, 88.2 mmol) were dissolved in 300 mL of THF, and K₂CO₃ (46.4 g, 336.0 mmol) was dissolved in 100 mL of H₂O and added. Pd(PPh₃)₄ (4.9 g, 4.2 mmol) was added thereto, and the mixture was stirred for 8 hours under an argon atmosphere. After the reaction was completed, the reaction solution was cooled to room temperature, then transferred to a separatory funnel, and the organic layer was separated. The separated organic solution was dried over MgSO₄, filtered, and concentrated. The resulting product was then dissolved in CH₂Cl₂, and n-hexane was added dropwise thereto to obtain 22.9 g of Compound E-1. (Yield: 78%, MS [M+H]⁺=350)

### 4-2) Synthesis of Compound E

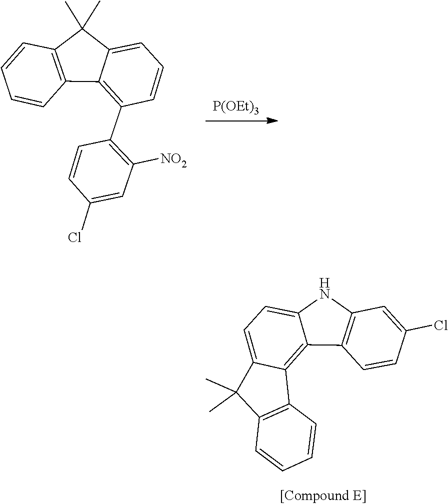 [SMILES: CC1(C)c2ccccc2-c2c(-c3ccc(Cl)cc3[N+](=O)[O-])cccc21 ; CC1(C)c2ccccc2-c2c1ccc1[nH]c3cc(Cl)ccc3c21 ; "P(OEt)3"]

Compound E-1 (20.0 g, 57.2 mmol) and triethyl phosphite (58.8 mL, 343.0 mmol) were added to a two-necked flask, and the mixture was stirred at 120° C. for 12 hours. After the reaction was completed, the reaction mixture was cooled to room temperature and added dropwise to distilled water. Ethyl acetate was then added to a separatory funnel and the organic layer was separated. The separated organic solution was dried over MgSO₄, and the sample was then purified by silica gel column chromatography to obtain 11.8 g of Compound E. (Yield: 65%, MS[M+H]⁺=318)

### Synthesis Example 5

### 5-1) Synthesis of Compound F-1

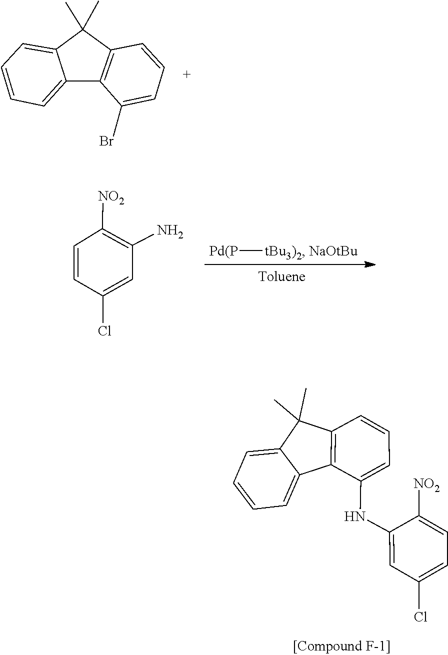

In a three-necked flask, 4-bromo-9,9-dimethyl-9H-fluorene (25.0 g, 91.5 mmol) and 5-chloro-2-nitroaniline (17.4 g, 100.7 mmol) were dissolved in 500 mL of toluene, and sodium tert-butoxide (13.2 g, 137.3 mmol) and Pd(P(t-Bu)₃)₂ (0.9 g, 1.8 mmol) were added, and the mixture was then stirred under reflux conditions for 6 hours under an argon atmosphere. After the reaction was completed, the reaction solution was cooled to room temperature, 200 mL of H₂O was added thereto, and then transferred to a separatory funnel and extracted. The extract was dried over MgSO₄ and concentrated. The sample was purified by silica gel column chromatography to obtain 22.0 g of Compound F-1. (Yield: 66%, MS [M+H]⁺=365)

### 5-2) Synthesis of Compound F-2

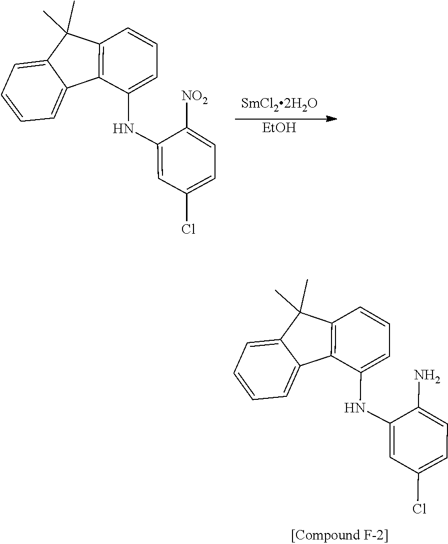 [SMILES: "EtOH" ; CC1(C)c2ccccc2-c2c(Nc3cc(Cl)ccc3[N+](=O)[O-])cccc21 ; CC1(C)c2ccccc2-c2c(Nc3cc(Cl)ccc3N)cccc21]

Compound F-1 (22.0 g, 60.3 mmol), tin (II) chloride dehydrate (40.8 g, 180.9 mmol), and EtOH (400 mL) were added to a two-necked flask, and the mixture was stirred under reflux conditions for 12 hours. After the reaction was completed, ethanol was distilled under reduced pressure, and then neutralized with a 1N NaOH solution to precipitate a solid. The precipitated solid was filtered and dissolved in toluene, then transferred to a separatory funnel, washed with water, and extracted. The extract was dried over MgSO₄, and concentrated to obtain 14.5 g of Compound F-2 as a solid. (Yield: 72%, MS [M+H]⁺=335)

### 5-3) Synthesis of Compound F

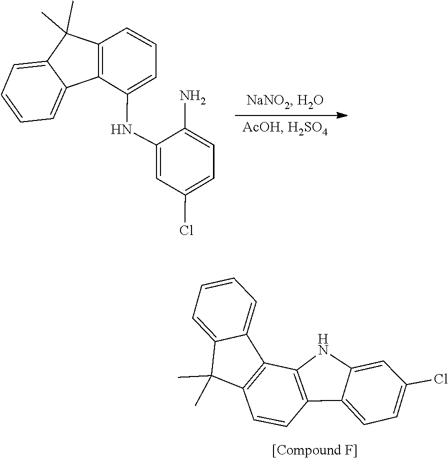 [SMILES: CC1(C)c2ccccc2-c2c(Nc3cc(Cl)ccc3N)cccc21 ; CC1(C)c2ccccc2-c2c1ccc1c2[nH]c2cc(Cl)ccc21]

Compound F-2 (14.5 g, 43.3 mmol), sulfuric acid (12 mL), and acetic acid (120 mL) were added to a three-necked flask, and the mixture was stirred at 10° C. for 10 minutes. Then, sodium nitrate (3.3 g, 47.6 mmol) was dissolved in 70 mL of distilled water and added dropwise for 15 minutes. After further stirring for 10 minutes, the mixture was stirred at 130° C. for 20 minutes. After the reaction was completed, the reaction mixture was cooled to room temperature, and 100 mL of H₂O was added thereto. The precipitated solid was filtered and washed with MeOH. The filtered solid was dissolved in CH₂Cl₂ and then dried over MgSO₄. The sample was purified by silica gel column chromatography to obtain 9.2 g of Compound F. (Yield: 67%, MS[M+H]⁺=318)

### Synthesis Example 6

### 6-1) Synthesis of Compound 1-1

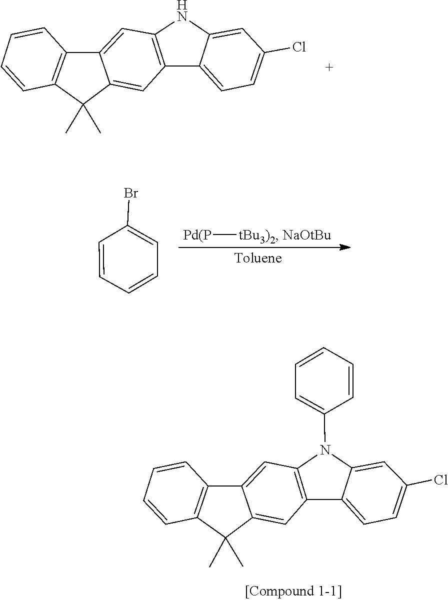

In a three-necked flask, Compound A (15.0 g, 47.2 mmol) and bromobenzene (7.8 g, 49.6 mmol) were dissolved in 300 mL of toluene, sodium tert-butoxide (6.8 g, 70.8 mmol) and Pd(P(t-Bu)₃)₂ (0.5 g, 0.9 mmol) were added thereto, and the mixture was stirred under reflux conditions for 9 hours under an argon atmosphere. After the reaction was completed, the reaction solution was cooled to room temperature, 200 mL of H₂O was added thereto, and then transferred to a separatory funnel and extracted. The extract was dried over MgSO₄ and concentrated. The sample was purified by silica gel column chromatography to obtain 12.1 g of Compound 1-1. (Yield: 70%, MS [M+H]⁺=365)

### 6-2) Synthesis of Compound 1

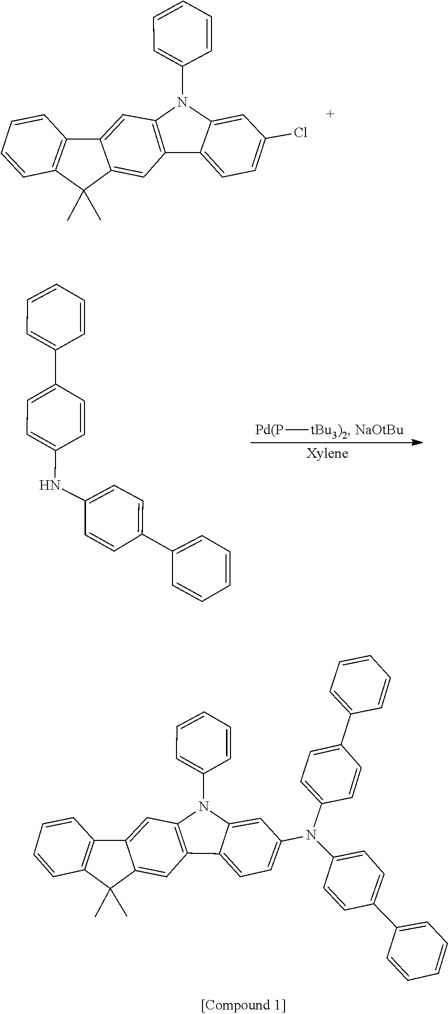

In a three-necked flask, Compound 1-1 (13.0 g, 47.6 mmol) and di([1,1′-biphenyl]-4-yl)amine (16.8 g, 52.3 mmol) were dissolved in 260 mL of xylene, sodium tert-butoxide (6.9 g, 71.4 mmol) and Pd(P(t-Bu)₃)₂ (0.5 g, 1.0 mmol) were added thereto, and the mixture was stirred under reflux conditions for 12 hours under an argon atmosphere. After the reaction was completed, the reaction solution was cooled to room temperature, 200 mL of H₂O was added thereto, and then transferred to a separatory funnel and extracted. The extract was dried over MgSO₄ and concentrated. The sample was purified by silica gel column chromatography and then purified by sublimation to obtain 11.0 g of Compound 1. (Yield: 34%, MS [M+H]⁺=679)

### Synthesis Example 7

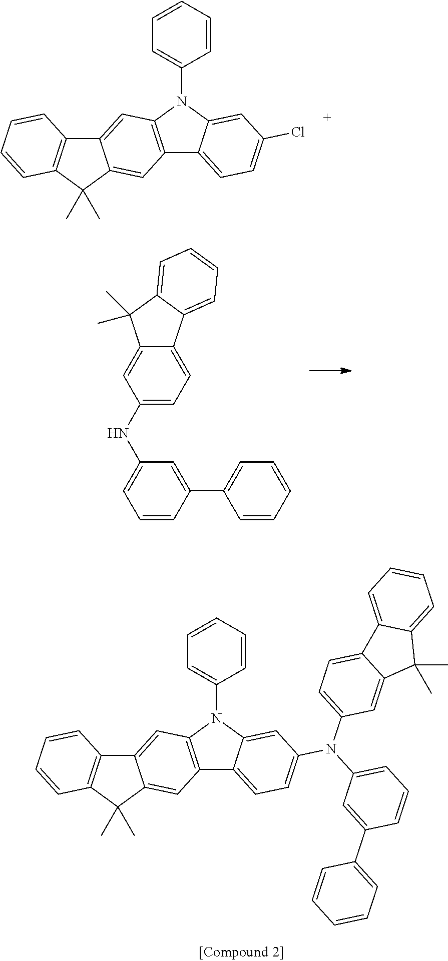 [SMILES: CC1(C)c2ccccc2-c2cc3c(cc21)c1ccc(Cl)cc1n3-c1ccccc1 ; CC1(C)c2ccccc2-c2ccc(Nc3cccc(-c4ccccc4)c3)cc21 ; CC1(C)c2ccccc2-c2ccc(N(c3cccc(-c4ccccc4)c3)c3ccc4c5cc6c(cc5n(-c5ccccc5)c4c3)-c3ccccc3C6(C)C)cc21]

Compound 2 was obtained in the same manner as in Synthesis Example 6, except that N-([1,1′-biphenyl]-3-yl)-9,9-dimethyl-9H-fluoren-2-amine was used instead of di([1,1′-biphenyl]-4-yl)amine in the preparation process of Synthesis Example 6.

### Synthesis Example 8

### 8-1) Synthesis of Compound 3-1

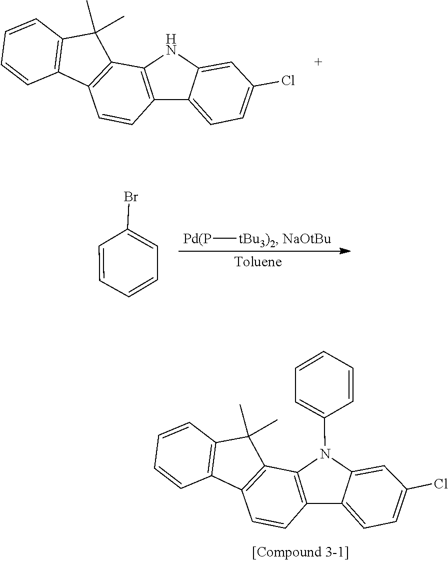

Compound 3-1 was obtained in the same manner as in Synthesis Example 6-1, except that Compound B was used instead of Compound A in the preparation process of Synthesis Example 6.

### 8-2) Synthesis of Compound 3

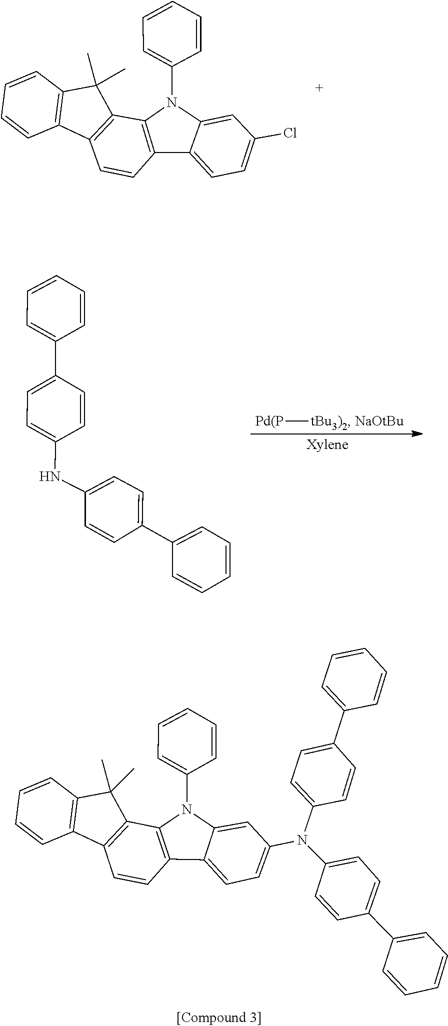

Compound 3 was obtained in the same manner as in Synthesis Example 6-2, except that Compound 3-1 was used instead of Compound 1-1 in the preparation process of Synthesis Example 6.

### Synthesis Example 9

### 9-1) Synthesis of Compound 4-1

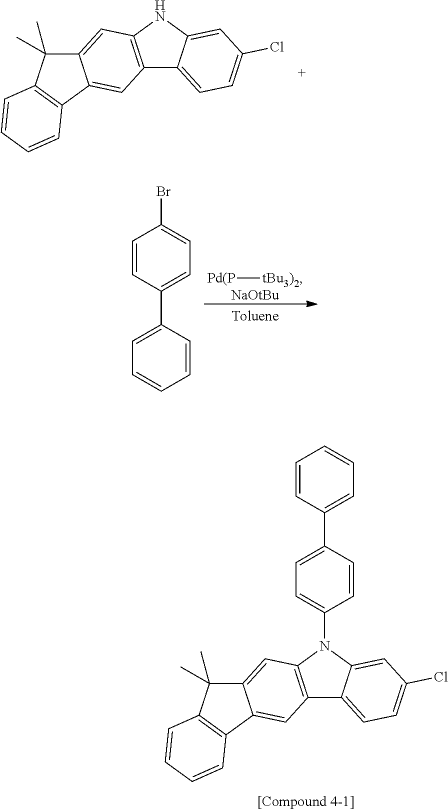

Compound 4-1 was obtained in the same manner as in Synthesis Example 6-1, except that Compound C was used instead of Compound A, and 4-bromo-1,1′-biphenyl was used instead of bromobenzene in the preparation process of Synthesis Example 6.

### 9-2) Synthesis of Compound 4

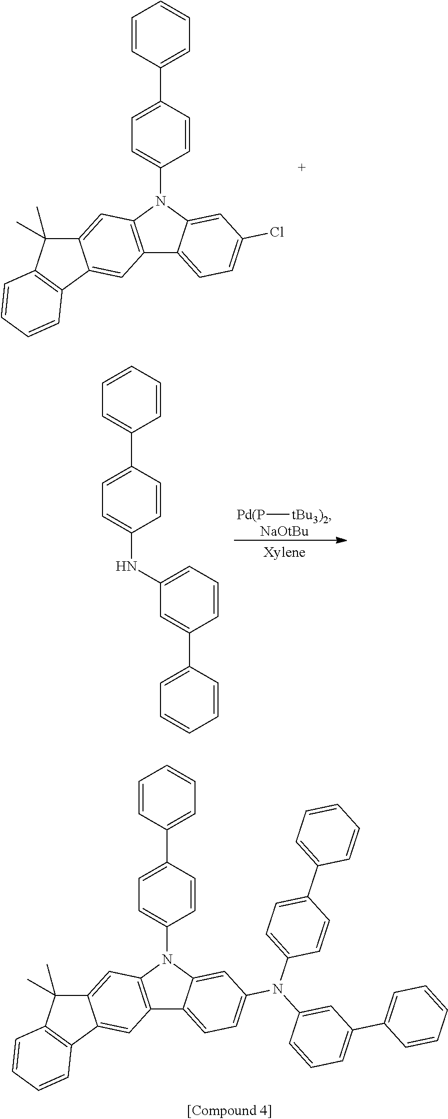

Compound 4 was obtained in the same manner as in Synthesis Example 6-2, except that Compound 4-1 was used instead of Compound 1-1, and N-([1,1′-biphenyl]-4-yl)-[1,1′-biphenyl]-3-amine was used instead of di([1,1′-biphenyl]-4-yl)amine in the preparation process of Synthesis Example 6.

### Synthesis Example 10

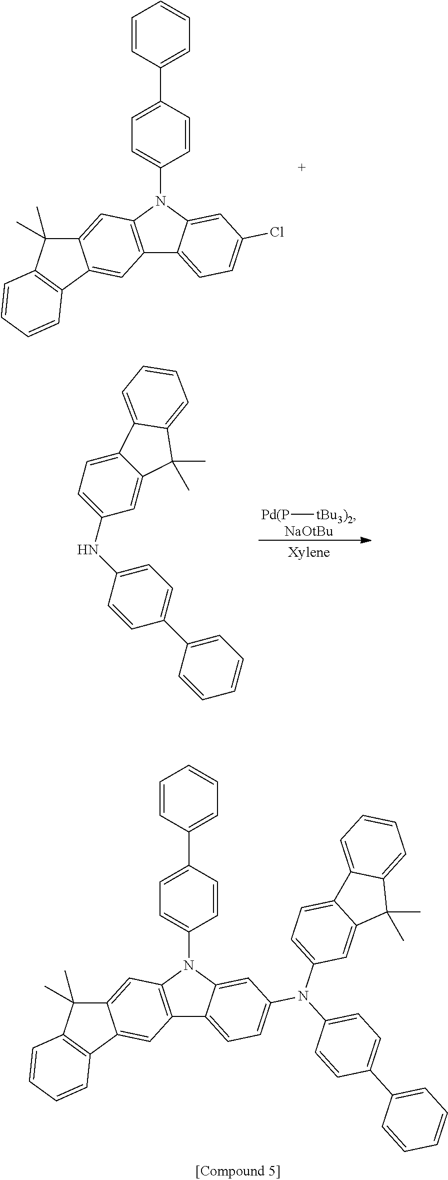

Compound 5 was obtained in the same manner as in Synthesis Example 6-2, except that Compound 4-1 was used instead of Compound 1-1, and N-([1,1′-biphenyl]-4-yl)-9,9-dimethyl-9H-fluoren-2-amine was used instead of di([1,1′-biphenyl]-4-yl)amine in the preparation process of Synthesis Example 6.

### Synthesis Example 11

### 11-1) Synthesis of Compound 6-1

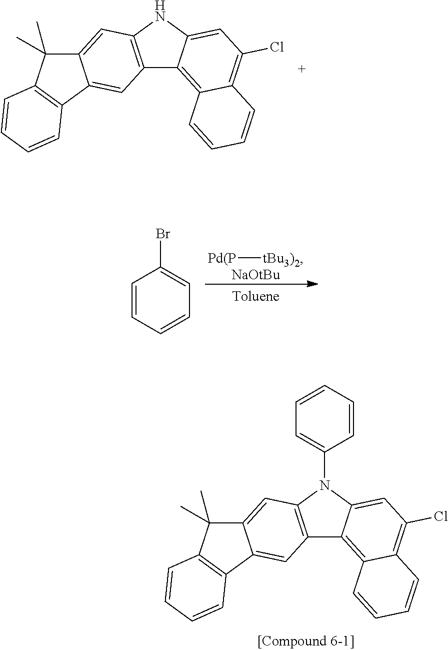

Compound 6-1 was obtained in the same manner as in Synthesis Example 6-1, except that Compound D was used instead of Compound A in the preparation process of Synthesis Example 6.

### 11-2) Synthesis of Compound 6

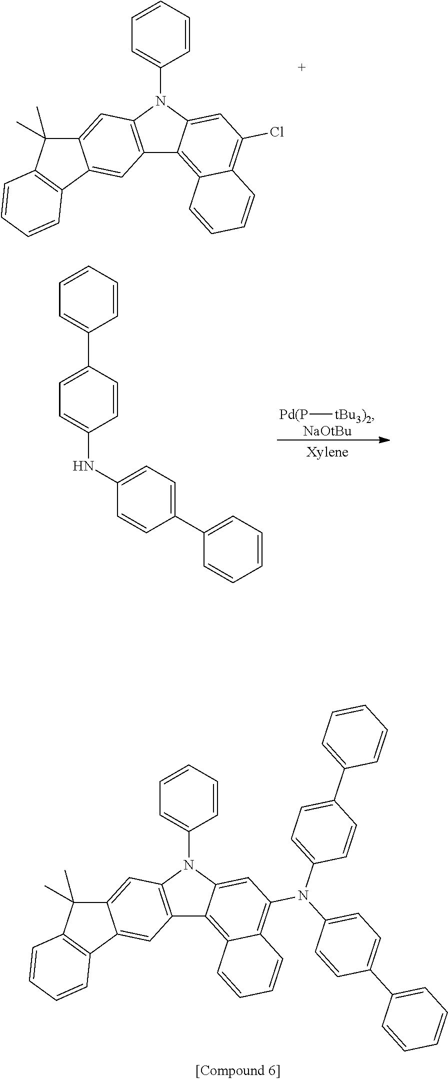

Compound 6 was obtained in the same manner as in Synthesis Example 6-2, except that Compound 6-1 was used instead of Compound 1-1 in the preparation process of Synthesis Example 6.

### Synthesis Example 12

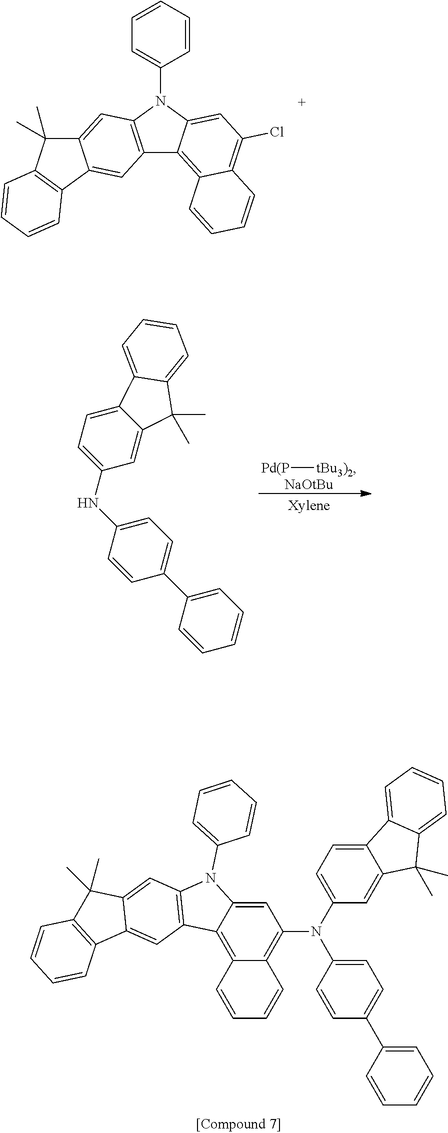

Compound 7 was obtained in the same manner as in Synthesis Example 6-2, except that Compound 6-1 was used instead of Compound 1-1, and N-([1,1′-biphenyl]-4-yl)-9,9-dimethyl-9H-fluoren-2-amine was used instead of di([1,1′-biphenyl]-4-yl)amine in the preparation process of Synthesis Example 6.

### Synthesis Example 13

### 13-1) Synthesis of Compound 8-1

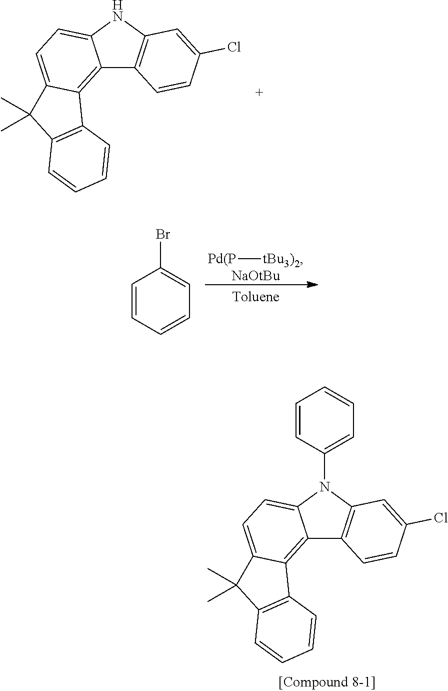

Compound 8-1 was obtained in the same manner as in Synthesis Example 6-1, except that Compound E was used instead of Compound A in the preparation process of Synthesis Example 6.

### 13-2) Synthesis of Compound 8

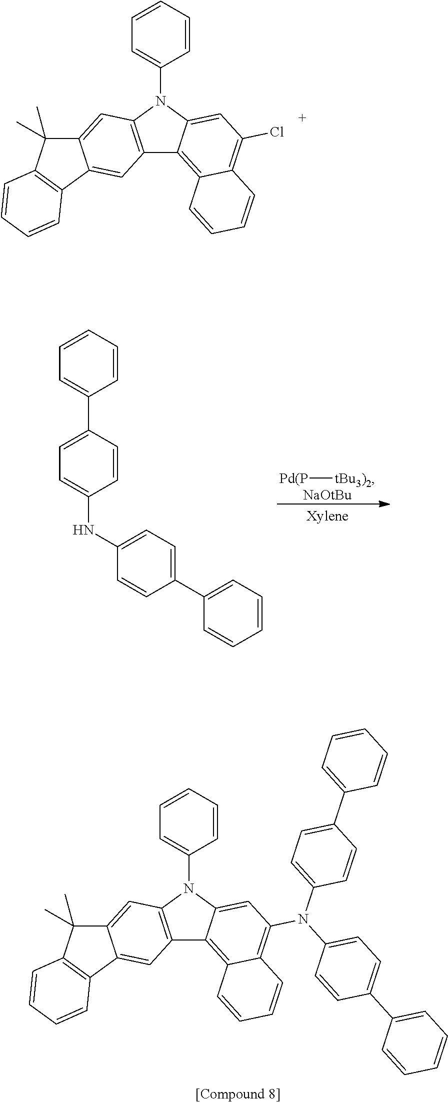

Compound 8 was obtained in the same manner as in Synthesis Example 6-2, except that Compound 8-1 was used instead of Compound 1-1 in the preparation process of Synthesis Example 6.

### Synthesis Example 14

### 14-1) Synthesis of Compound 9-1

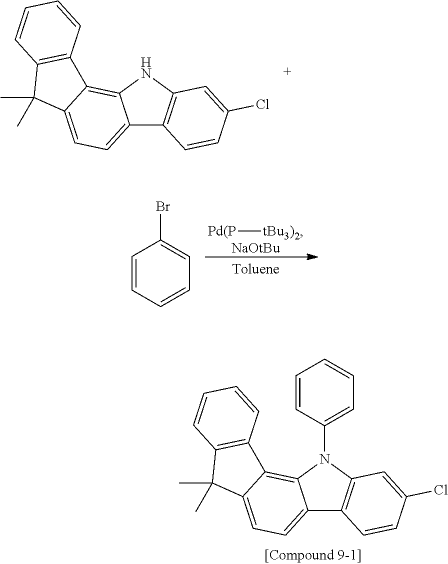

Compound 9-1 was obtained in the same manner as in Synthesis Example 6-1, except that Compound F was used instead of Compound A in the preparation process of Synthesis Example 6.

### 14-2) Synthesis of Compound 9

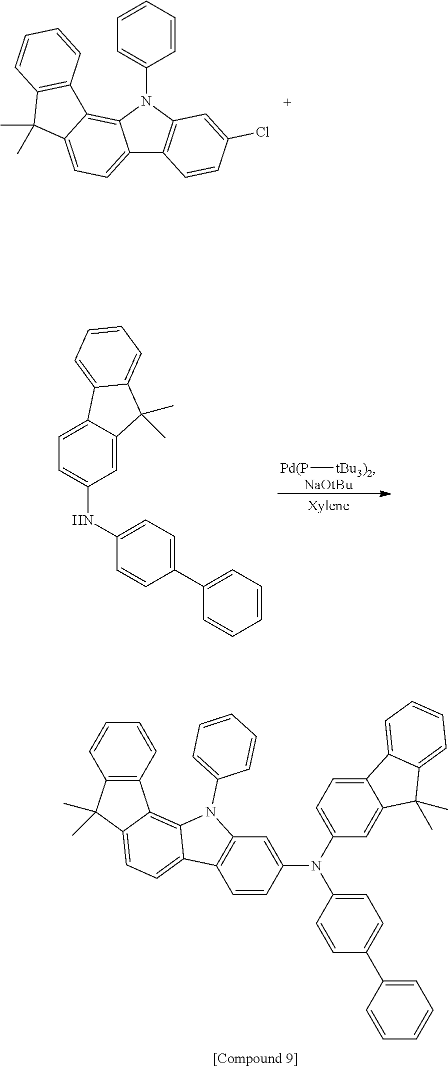

Compound 9 was obtained in the same manner as in Synthesis Example 6-2, except that Compound 9-1 was used instead of Compound 1-1, and N-([1,1′-biphenyl]-4-yl)-9,9-dimethyl-9H-fluoren-2-amine was used instead of di([1,1′-biphenyl]-4-yl)amine in the preparation process of Synthesis Example 6.

### Synthesis Example 15

### 15-1) Synthesis of Compound 10-1

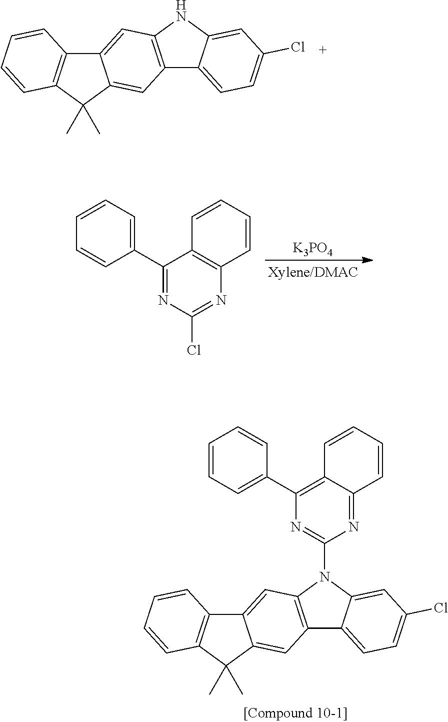

Compound A (12.0 g, 37.8 mmol), 2-chloro-4-phenylquinazoline (9.5 g, 39.6 mmol), K₃PO₄ (12.0 g, 56.6 mmol), xylene (180 mL), and DMAC (60 mL) were added to a three-necked flask, and the mixture was stirred under reflux conditions for 8 hours under an argon atmosphere. After the reaction was completed, the reaction solution was cooled to room temperature, 200 mL of H₂O was added thereto, and then transferred to a separatory funnel and extracted. The extract was dried over MgSO₄ and concentrated. The sample was purified by silica gel column chromatography to obtain 13.4 g of Compound 10-1. (Yield: 68%, MS [M+H]⁺=522)

### 15-2) Synthesis of Compound 10

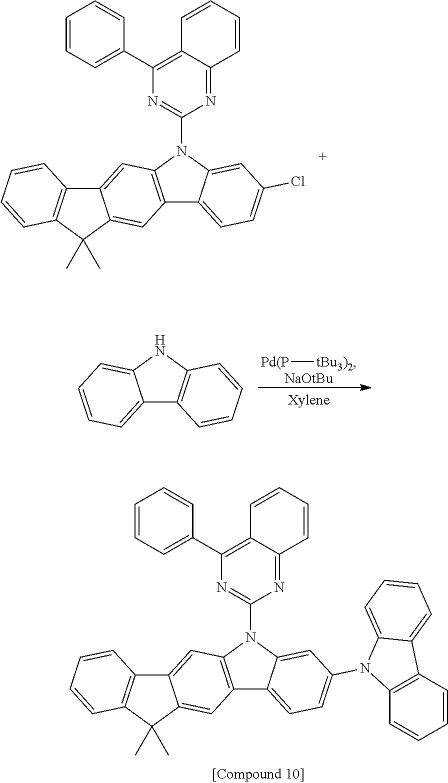

In a three-necked flask, Compound 10-1 (13.0 g, 24.9 mmol) and 9H-carbazole (4.6 g, 27.4 mmol) were dissolved in 400 mL of xylene, sodium t-butoxide (3.6 g, 37.4 mmol) and Pd(P(t-Bu)₃)₂ (0.3 g, 0.5 mmol) were added thereto, and the mixture was stirred under reflux conditions for 12 hours under an argon atmosphere. After the reaction was completed, the reaction solution was cooled to room temperature, 200 mL of H₂O was added thereto, and then transferred to a separatory funnel and extracted. The extract was dried over MgSO₄ and concentrated. The sample was purified by silica gel column chromatography and then purified by sublimation to obtain 5.4 g of Compound 10. (Yield: 32%, MS [M+H]⁺=679)

### Synthesis Example 16

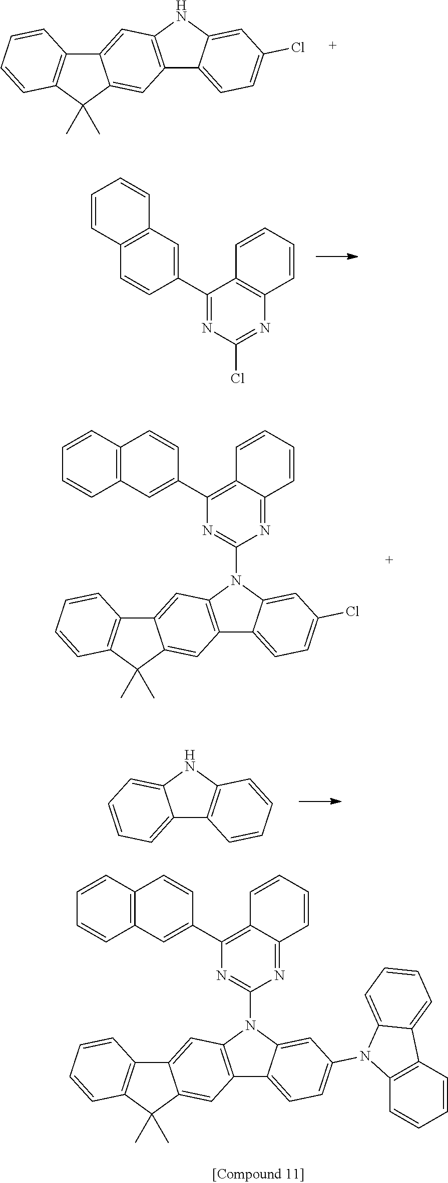

Compound 11 was obtained in the same manner as in Synthesis Example 15, except that 2-chloro-4-(naphthalen-2-yl)quinazoline was used instead of 2-chloro-4-phenylquinazoline in the preparation process of Synthesis Example 15.

### Synthesis Example 17

### 17-1) Synthesis of Compound 12-1

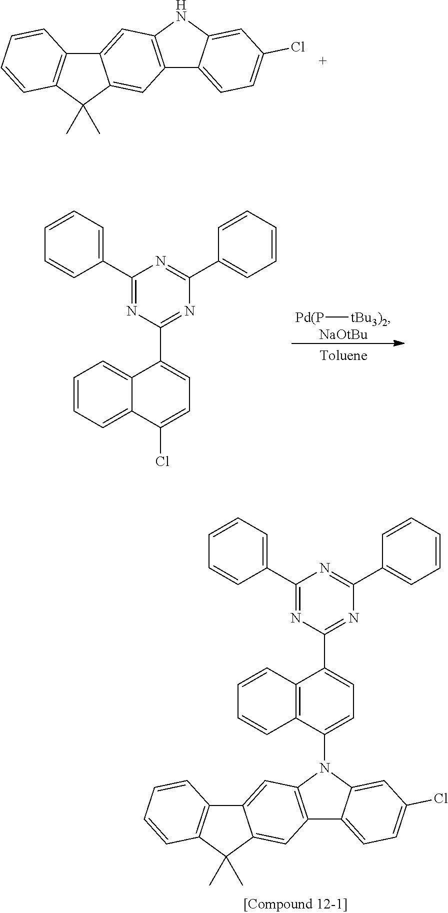

In a three-necked flask, Compound A (15.0 g, 47.2 mmol) and 2-(4-chloronaphthalen-1-yl)-4,6-diphenyl-1,3,5-triazine (13.0 g, 33.0 mmol) were dissolved in 400 mL of toluene, sodium t-butoxide (4.5 g, 47.2 mmol) and Pd(P(t-Bu)₃)₂ (0.3 g, 0.6 mmol) were added thereto, and the mixture was stirred under reflux conditions for 7 hours under an argon atmosphere. After the reaction was completed, the reaction solution was cooled to room temperature, 150 mL of H₂O was added thereto, and then transferred to a separatory funnel and extracted. The extract was dried over MgSO₄ and concentrated. The sample was purified by silica gel column chromatography and then purified by sublimation to obtain 11.7 g of Compound 12-1. (Yield: 55%, MS [M+H]⁺=394)

### 17-2) Synthesis of Compound 12

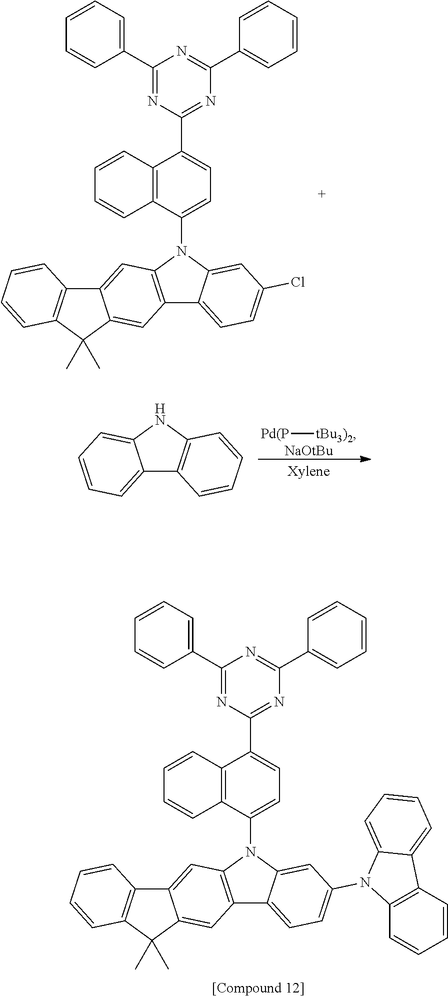

Compound 12 was obtained in the same manner as in Synthesis Example 15-2, except that Compound 12-1 was used instead of Compound 10-1 in the preparation process of Synthesis Example 15.

### Synthesis Example 18

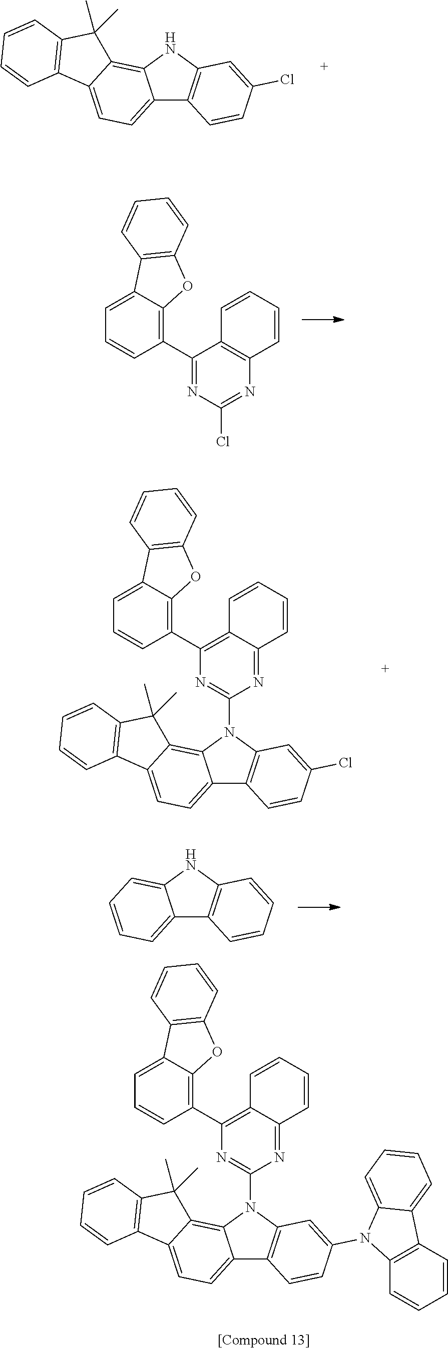

Compound 13 was obtained in the same manner as in Synthesis Example 15, except that Compound B was used instead of Compound A, and 2-chloro-4-(dibenzo[b,d]furan-4-yl)quinazoline was used instead of 2-chloro-4-phenylquinazoline in the preparation process of Synthesis Example 15.

### Synthesis Example 19

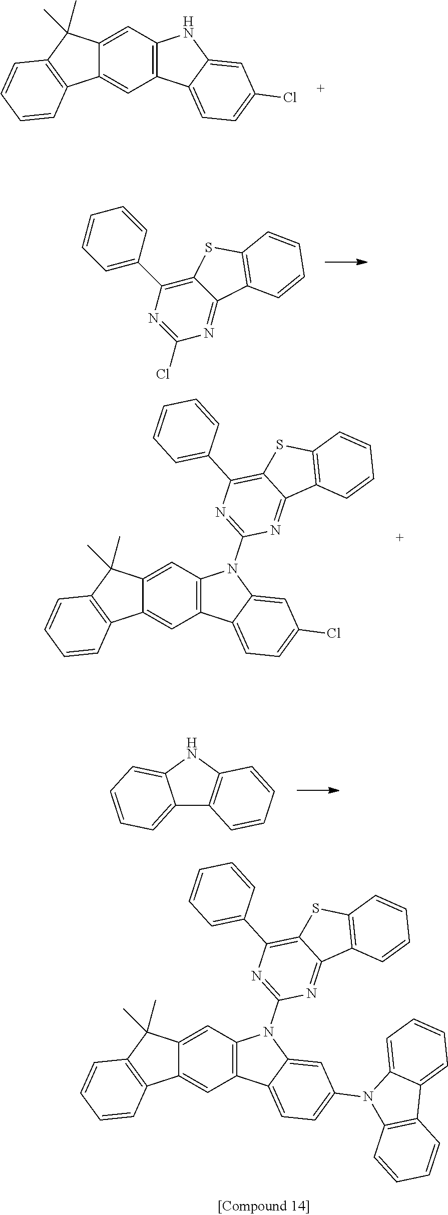

Compound 14 was obtained in the same manner as in Synthesis Example 17, except that Compound C was used instead of Compound A, and 2-chloro-4-phenylbenzo[4,5]thieno[3,2-d]pyrimidine was used instead of 2-(4-chloronaphthalen-1-yl)-4,6-diphenyl-1,3,5-triazine in the preparation process of Synthesis Example 17.

### Synthesis Example 20

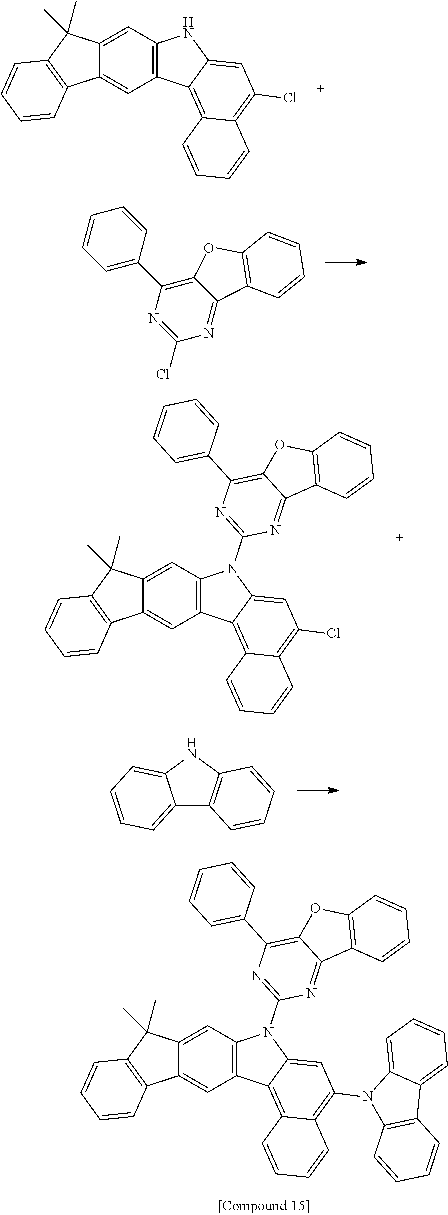

Compound 15 was obtained in the same manner as in Synthesis Example 17, except that Compound D was used instead of Compound A, and 2-chloro-4-phenylbenzo[4,5]furo[3,2-d]pyrimidine was used instead of 2-(4-chloronaphthalen-1-yl)-4,6-diphenyl-1,3,5-triazine in the preparation process of Synthesis Example 17.

### Synthesis Example 21

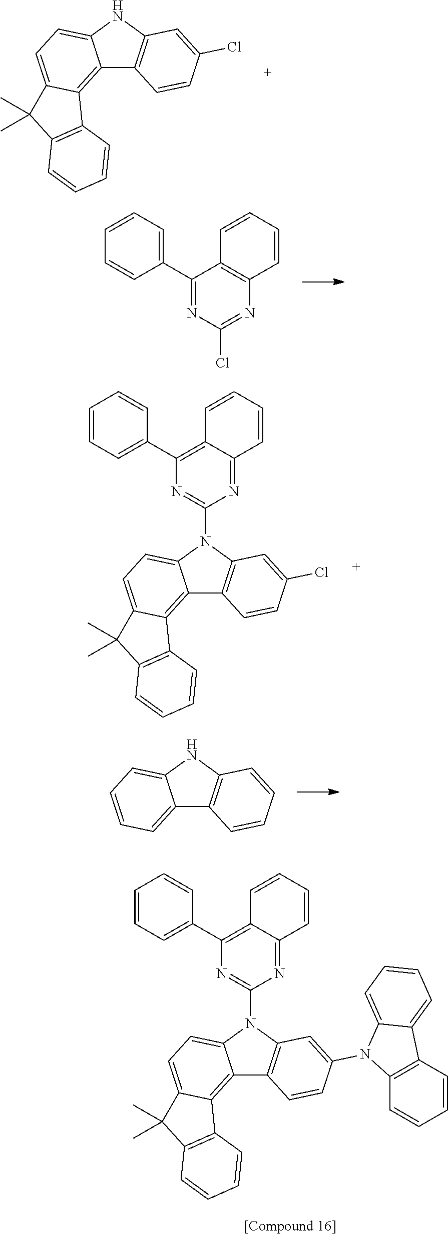

Compound 16 was obtained in the same manner as in Synthesis Example 15, except that Compound E was used instead of Compound A in the preparation process of Synthesis Example 15.

### Synthesis Example 22

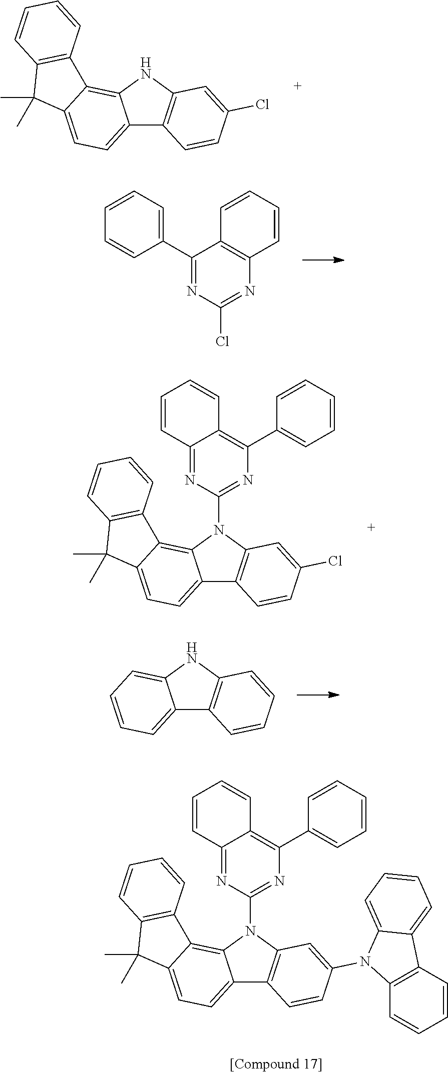 [SMILES: CC1(C)c2ccccc2-c2c1ccc1c2[nH]c2cc(Cl)ccc21 ; Clc1nc(-c2ccccc2)c2ccccc2n1 ; CC1(C)c2ccccc2-c2c1ccc1c3ccc(Cl)cc3n(-c3nc(-c4ccccc4)c4ccccc4n3)c21 ; c1ccc2c(c1)[nH]c1ccccc12 ; CC1(C)c2ccccc2-c2c1ccc1c3ccc(-n4c5ccccc5c5ccccc54)cc3n(-c3nc(-c4ccccc4)c4ccccc4n3)c21]

Compound 17 was obtained in the same manner as in Synthesis Example 15, except that Compound F was used instead of Compound A in the preparation process of Synthesis Example 15.

### EXAMPLES

### Comparative Example 1

A glass substrate on which ITO (indium tin oxide) was coated as a thin film to a thickness of 1400 Å was put into distilled water in which a detergent was dissolved, and ultrasonically cleaned. In this case, a product manufactured by Fischer Co., was used as the detergent, and as the distilled water, distilled water filtered twice using a filter manufactured by Millipore Co., was used. After the ITO was cleaned for 30 minutes, ultrasonic cleaning was repeated twice using distilled water for 10 minutes. After the cleaning with distilled water was completed, the substrate was ultrasonically cleaned with solvents of isopropyl alcohol, acetone, and methanol, then dried and transferred to a plasma cleaner. In addition, the substrate was cleaned for 5 minutes using oxygen plasma, and then transferred to a vacuum depositor.

On the ITO transparent electrode thus prepared, the following compound [HI-A] and the following compound [HAT] were sequentially deposited under thermal vacuum deposition in a thickness of 800 Å and 50 Å, respectively, to form a hole injection layer.

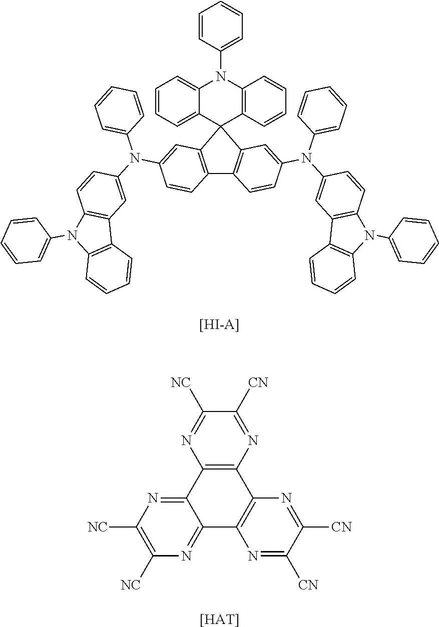

The following compound [HT-A] was vacuum-deposited thereon as a hole transporting layer to a thickness of 800 Å, and then the following compound [EB-A] was vapor deposited as an electron blocking layer to a thickness of 600 Å.

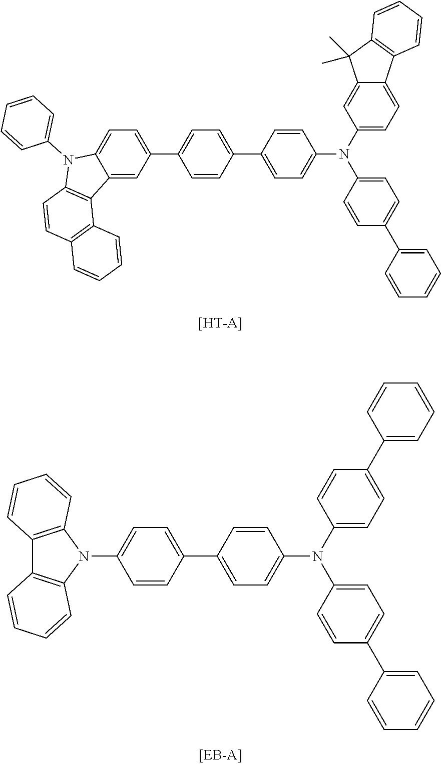

Subsequently, the following host compound [RH-A] and a 2% dopant compound [RD] were vacuum deposited as a light emitting layer to a thickness of 400 Å.

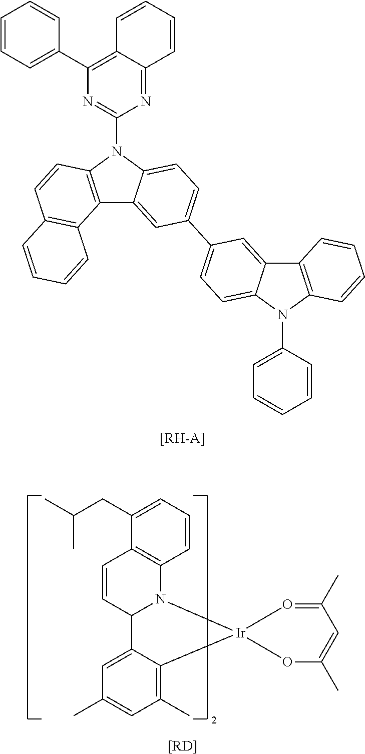

Then, the following compounds [ET-A] and [LiQ] were thermally vacuum deposited at a ratio of 1:1 as electron transport and injection layers to a thickness of 360 Å, and then the following compound [LiQ] was vacuum deposited to a thickness of 5 Å.

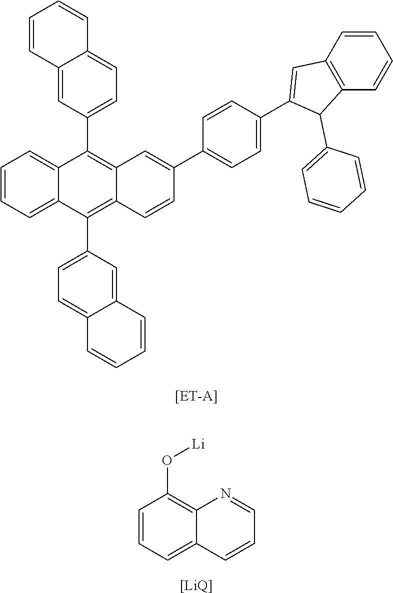

Magnesium and silver were sequentially deposited on the electron injecting layer at a ratio of 10:1 to a thickness of 220 Å and aluminum to a thickness of 1000 Å to form a cathode, thereby manufacturing an organic light emitting device.

### Examples 1-9 and Comparative Examples 2-8

The organic light emitting devices of Examples 1 to 9 and Comparative Examples 2 to 8 were respectively manufactured in the same manner as in Comparative Example 1, except that Compounds 1 to 9 shown in Table 1 below and the following compounds [EB-B] to [EB-H] were used as electron blocking layers. The voltage, efficiency, and lifetime were measured by applying a current to the organic light emitting devices manufactured in Examples 1-9 and Comparative Examples 2-8, and the results are shown in Table 1 below. At this time, the voltage and the efficiency were measured by applying a current density of 10 mA/cm² (@10 mA/cm²), LT₉₈ means the time required for the initial luminance to decrease to 98% of its initial value at a current density of 20 mA/cm² (@20 mA/cm²).

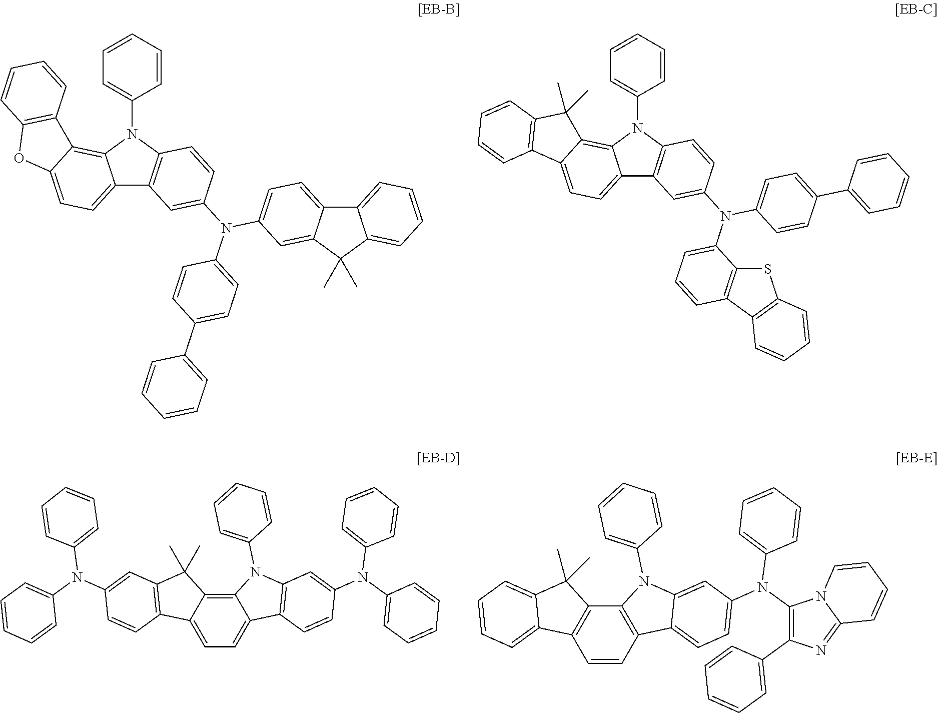

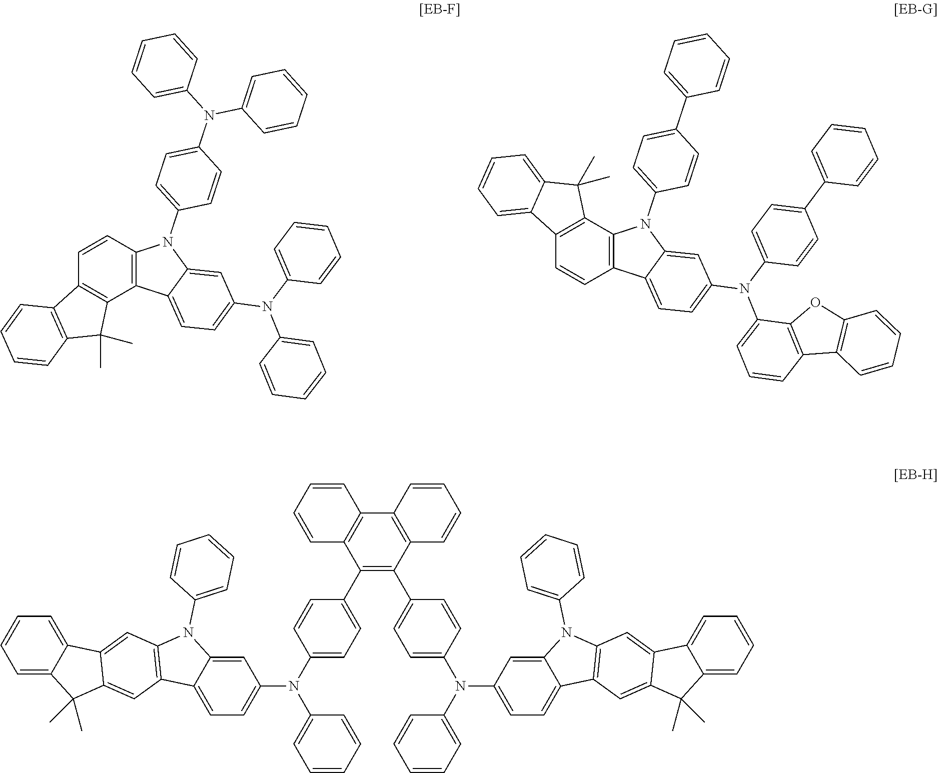 [SMILES: CC1(C)c2ccccc2-c2ccc3c(c21)c1ccc(N(c2ccccc2)c2ccccc2)cc1n3-c1ccc(N(c2ccccc2)c2ccccc2)cc1 ; CC1(C)c2ccccc2-c2ccc3c4ccc(N(c5ccc(-c6ccccc6)cc5)c5cccc6c5oc5ccccc56)cc4n(-c4ccc(-c5ccccc5)cc4)c3c21 ; CC1(C)c2ccccc2-c2cc3c(cc21)c1ccc(N(c2ccccc2)c2ccc(-c4c(-c5ccc(N(c6ccccc6)c6ccc7c8cc9c(cc8n(-c8ccccc8)c7c6)-c6ccccc6C9(C)C)cc5)c5ccccc5c5ccccc45)cc2)cc1n3-c1ccccc1]

TABLE 1

| | Electron | | | | | |
|---|---|---|---|---|---|---|
| | blocking | | | | | @20 |
| | layer | @10 mA/cm² | | | | mA/cm² |
| | material | V | cd/A | CIE-x | CIE-y | LT₉₈ (h) |
| Example 1 | Compound 1 | 5.09 | 22.93 | 0.661 | 0.337 | 94 |
| Example 2 | Compound 2 | 5.07 | 23.12 | 0.661 | 0.338 | 85 |
| Example 3 | Compound 3 | 4.98 | 22.87 | 0.659 | 0.337 | 83 |
| Example 4 | Compound 4 | 5.01 | 22.15 | 0.660 | 0.336 | 101 |
| Example 5 | Compound 5 | 4.89 | 22.11 | 0.658 | 0.337 | 84 |
| Example 6 | Compound 6 | 4.95 | 23.31 | 0.661 | 0.338 | 90 |
| Example 7 | Compound 7 | 5.03 | 22.75 | 0.662 | 0.338 | 76 |
| Example 8 | Compound 8 | 5.00 | 22.67 | 0.659 | 0.337 | 91 |
| Example 9 | Compound 9 | 4.93 | 22.57 | 0.651 | 0.336 | 88 |
| Comparative | EB-A | 5.23 | 20.93 | 0.658 | 0.339 | 56 |
| Example 1 | | | | | | |
| Comparative | EB-B | 5.31 | 18.13 | 0.655 | 0.338. | 48 |
| Example 2 | | | | | | |
| Comparative | EB-C | 5.53 | 17.61 | 0.651 | 0.340 | 22 |
| Example 3 | | | | | | |
| Comparative | EB-D | 6.26 | 10.10 | 0.648 | 0.340 | 3 |
| Example 4 | | | | | | |
| Comparative | EB-E | 5.78 | 15.15 | 0.650 | 0.340 | 20 |
| Example 5 | | | | | | |
| Comparative | EB-F | 6.25 | 9.13 | 0.653 | 0.342 | 5 |
| Example 6 | | | | | | |
| Comparative | EB-G | 5.63 | 18.61 | 0.651 | 0.340 | 35 |
| Example 7 | | | | | | |
| Comparative | EB-H | 7.53 | 6.13 | 0.645 | 0.340 | 5 |
| Example 8 | | | | | | |

### Examples 10 to 17 and Comparative Examples 9 to 13

The organic light emitting devices of Examples 10 to 17 and Comparative Examples 9 to 13 were respectively manufactured in the same manner as in Comparative Example 1, except that Compounds 10 to 17 shown in Table 2 below or the following compounds [RH-B] to [RH-E] were used as respective host materials instead of the compound [RH-A] in Comparative Example 1.

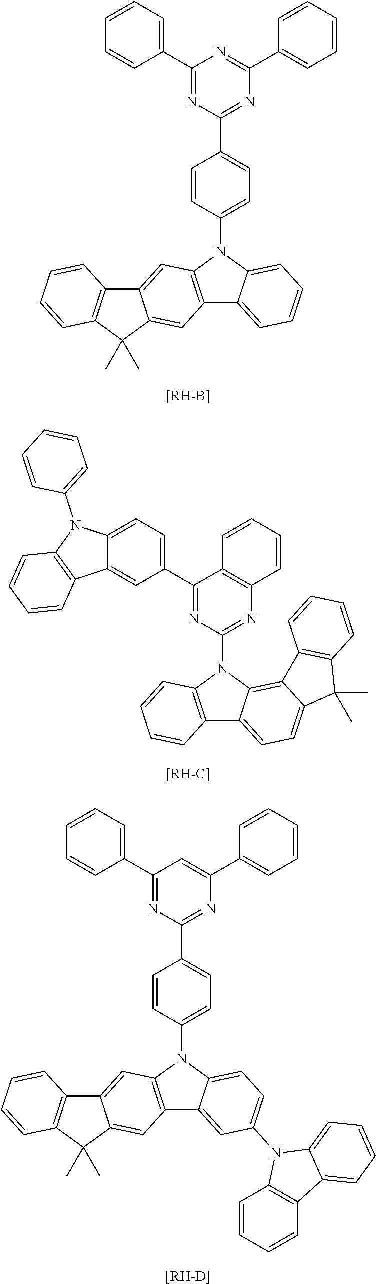

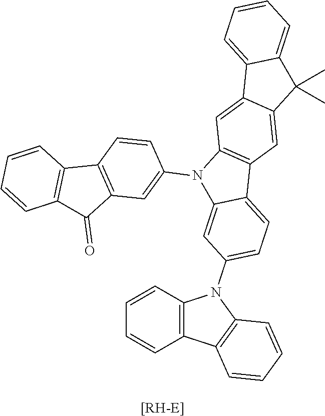

The voltage, efficiency, and lifetime were measured by applying a current to the organic light emitting devices manufactured in the Examples 10-17 and Comparative Examples 9-13, and the results are shown in Table 2 below.

TABLE 2

| | | | | | | @20 |
|---|---|---|---|---|---|---|
| | | | | | | mA/cm² |
| | Host | @10 mA/cm² | | | | LT₉₈ |
| | material | V | cd/A | CIE-x | CIE-y | (h) |
| Example 10 | Compound 10 | 4.75 | 24.51 | 0.660 | 0.337 | 101 |
| Example 11 | Compound 11 | 4.66 | 24.33 | 0.661 | 0.336 | 103 |
| Example 12 | Compound 12 | 4.77 | 23.42 | 0.659 | 0.337 | 98 |
| Example 13 | Compound 13 | 4.61 | 24.16 | 0.661 | 0.337 | 110 |
| Example 14 | Compound 14 | 4.70 | 22.76 | 0.661 | 0.336 | 91 |
| Example 15 | Compound 15 | 4.73 | 24.73 | 0.658 | 0.336 | 81 |
| Example 16 | Compound 16 | 4.80 | 24.91 | 0.660 | 0.338 | 106 |
| Example 17 | Compound 17 | 4.81 | 23.76 | 0.661 | 0.337 | 95 |
| Comparative | RH-A | 5.23 | 20.93 | 0.658 | 0.339 | 56 |
| Example 9 | | | | | | |
| Comparative | RH-B | 5.12 | 17.51 | 0.658 | 0.339 | 54 |
| Example 10 | | | | | | |
| Comparative | RH-C | 5.72 | 24.36 | 0.651 | 0.340 | 20 |
| Example 11 | | | | | | |
| Comparative | RH-D | 5.37 | 19.87 | 0.655 | 0.337 | 31 |
| Example 12 | | | | | | |
| Comparative | RH-E | 6.27 | 13.57 | 0.653 | 0.342 | 15 |
| Example 13 | | | | | | |

### EXPLANATION OF SIGNS

| | |
|---|---|
| 1: substrate | 2: anode |
| 3: light emitting layer | 4: cathode |
| 5: hole injection layer | 6: hole transport layer |
| 7: light emitting layer | 8: electron transport layer |

1. A compound of Chemical Formula 1: 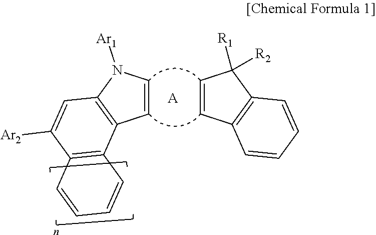
wherein, in Chemical Formula 1:
A is a benzene ring fused to two adjacent rings;
R₁ and R₂ are each independently a substituted or unsubstituted C₁₋₆₀ alkyl, or a substituted or unsubstituted C₆₋₆₀ aryl; and
Ar₁ is phenyl, biphenyl, naphthyl, or any one of the following Chemical Formulae 2-1 to 2-5: 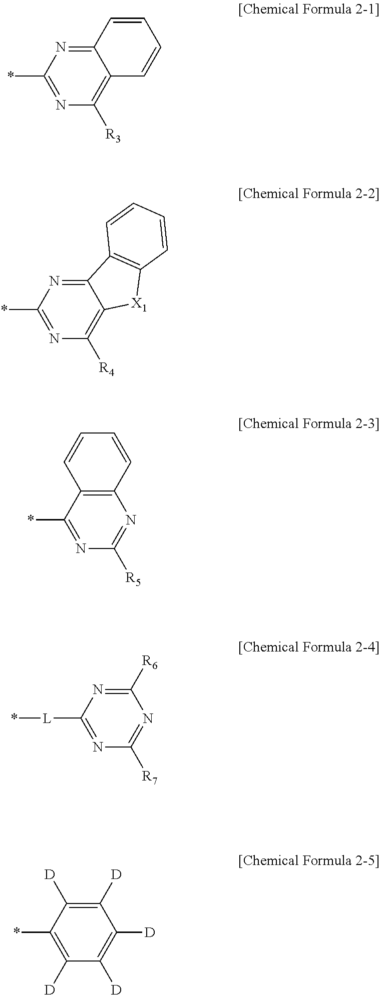
wherein, in Chemical Formulae 2-1, 2-2, 2-3, 2-4, and 2-5:
R₃, R₄, R₅, R₆, and R₇ are each independently phenyl, biphenyl, or naphthyl;
X₁ is O or S;
L is a single bond or phenylene;
Ar₂ is 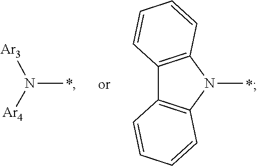
Ar₃ and Ar₄ are each independently a substituted or unsubstituted C₆₋₆₀ aryl; and
n is 0 or 1.

2. The compound according to claim 1, wherein the compound of Chemical Formula 1 is any one of the following Chemical Formulae 1-1, 1-2, 1-3, 1-4, 1-5, or 1-6: 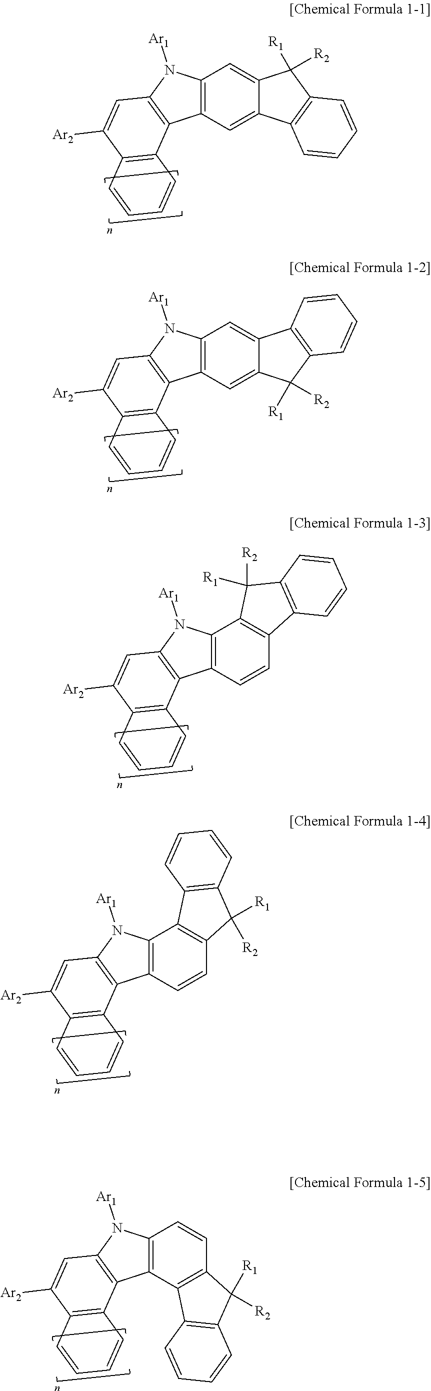 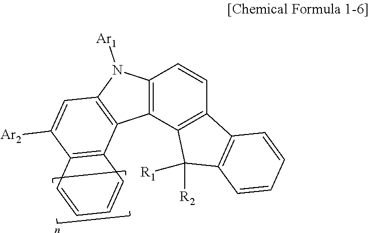
wherein, in Chemical Formulae 1-1, 1-2, 1-3, 1-4, 1-5, and 1-6;
R₁, R₂, Ar₁, Ar₂, and n are as defined in claim 1.

3. The compound according to claim 1, wherein R₁ and R₂ are methyl or phenyl.

4. The compound according to claim 1, wherein Ar₃ and Ar₄ are phenyl, biphenyl, terphenyl, or dimethylfluorenyl.

5. The compound according to claim 1, wherein the compound of Chemical Formula 1 is any one compound selected from the group consisting of the following compounds:

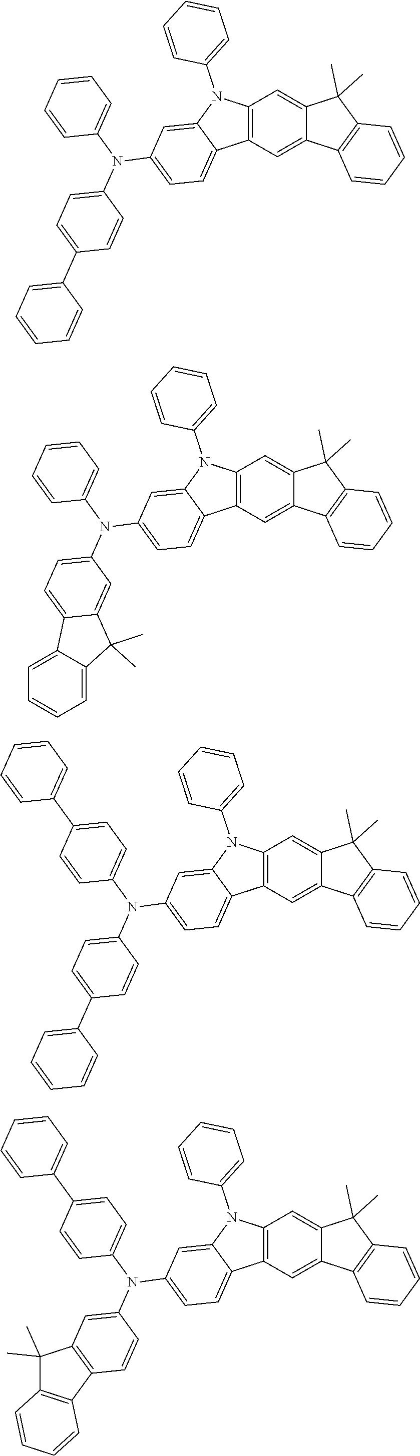

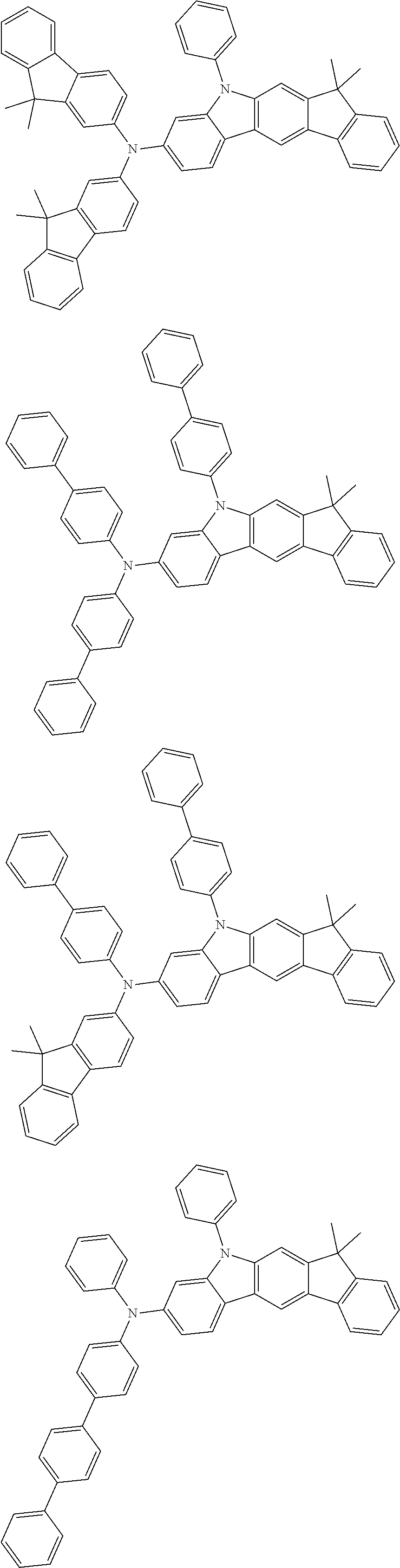 [SMILES: CC1(C)c2ccccc2-c2ccc(N(c3ccc4c(c3)C(C)(C)c3ccccc3-4)c3ccc4c5cc6c(cc5n(-c5ccccc5)c4c3)C(C)(C)c3ccccc3-6)cc21 ; CC1(C)c2ccccc2-c2cc3c4ccc(N(c5ccc(-c6ccccc6)cc5)c5ccc(-c6ccccc6)cc5)cc4n(-c4ccc(-c5ccccc5)cc4)c3cc21 ; CC1(C)c2ccccc2-c2ccc(N(c3ccc(-c4ccccc4)cc3)c3ccc4c5cc6c(cc5n(-c5ccc(-c7ccccc7)cc5)c4c3)C(C)(C)c3ccccc3-6)cc21 ; CC1(C)c2ccccc2-c2cc3c4ccc(N(c5ccccc5)c5ccc(-c6ccc(-c7ccccc7)cc6)cc5)cc4n(-c4ccccc4)c3cc21]

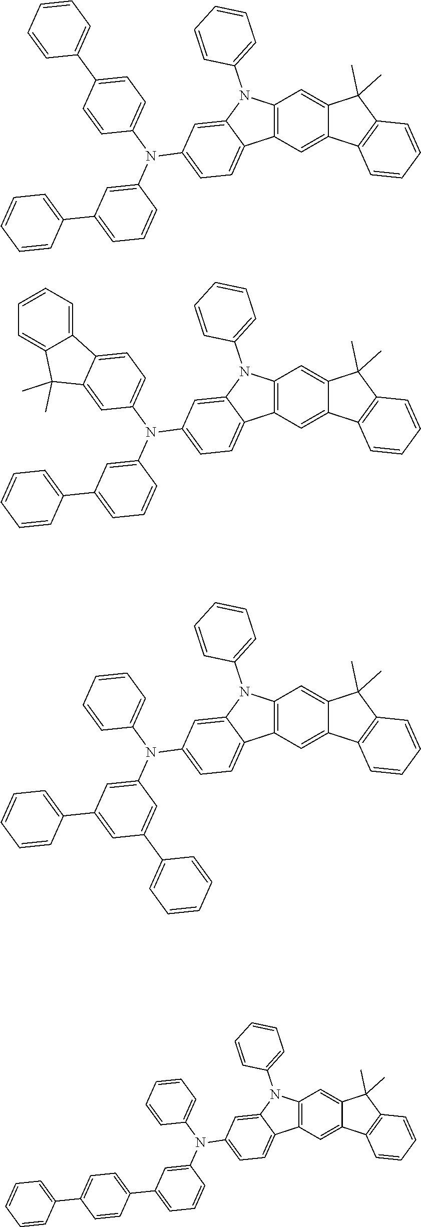

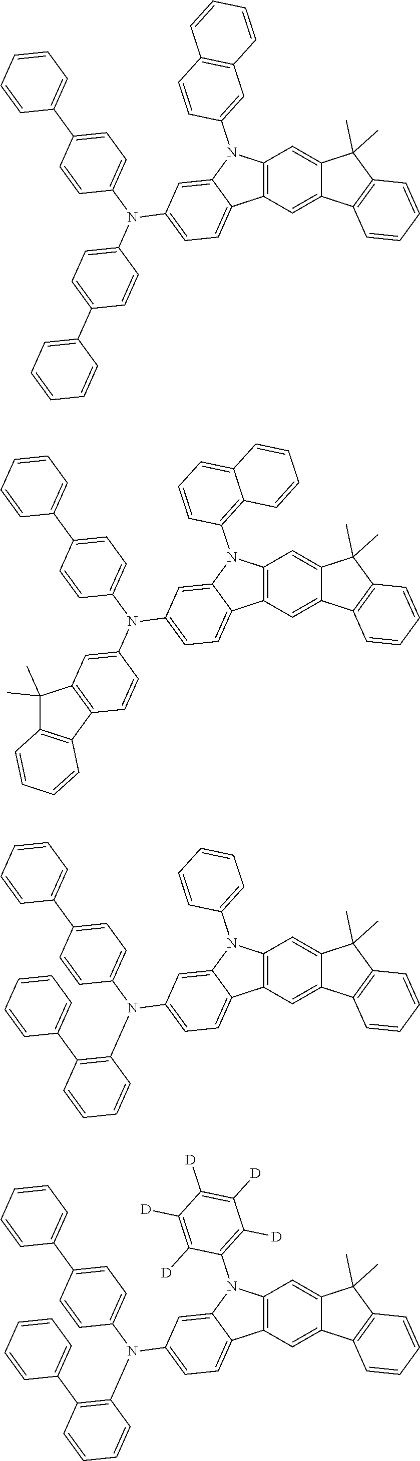 [SMILES: CC1(C)c2ccccc2-c2cc3c4ccc(N(c5ccc(-c6ccccc6)cc5)c5ccc(-c6ccccc6)cc5)cc4n(-c4ccc5ccccc5c4)c3cc21 ; CC1(C)c2ccccc2-c2ccc(N(c3ccc(-c4ccccc4)cc3)c3ccc4c5cc6c(cc5n(-c5cccc7ccccc57)c4c3)C(C)(C)c3ccccc3-6)cc21 ; CC1(C)c2ccccc2-c2cc3c4ccc(N(c5ccc(-c6ccccc6)cc5)c5ccccc5-c5ccccc5)cc4n(-c4ccccc4)c3cc21 ; [2H]c1c([2H])c([2H])c(-n2c3cc(N(c4ccc(-c5ccccc5)cc4)c4ccccc4-c4ccccc4)ccc3c3cc4c(cc32)C(C)(C)c2ccccc2-4)c([2H])c1[2H]]

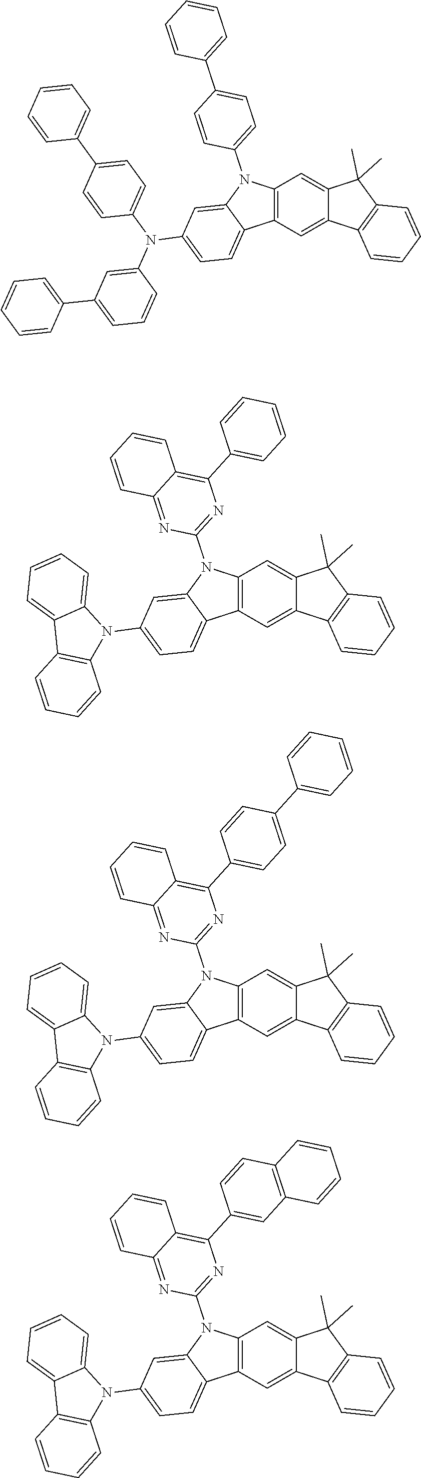 [SMILES: CC1(C)c2ccccc2-c2cc3c4ccc(N(c5ccc(-c6ccccc6)cc5)c5cccc(-c6ccccc6)c5)cc4n(-c4ccc(-c5ccccc5)cc4)c3cc21 ; CC1(C)c2ccccc2-c2cc3c4ccc(-n5c6ccccc6c6ccccc65)cc4n(-c4nc(-c5ccccc5)c5ccccc5n4)c3cc21 ; CC1(C)c2ccccc2-c2cc3c4ccc(-n5c6ccccc6c6ccccc65)cc4n(-c4nc(-c5ccc(-c6ccccc6)cc5)c5ccccc5n4)c3cc21 ; CC1(C)c2ccccc2-c2cc3c4ccc(-n5c6ccccc6c6ccccc65)cc4n(-c4nc(-c5ccc6ccccc6c5)c5ccccc5n4)c3cc21]

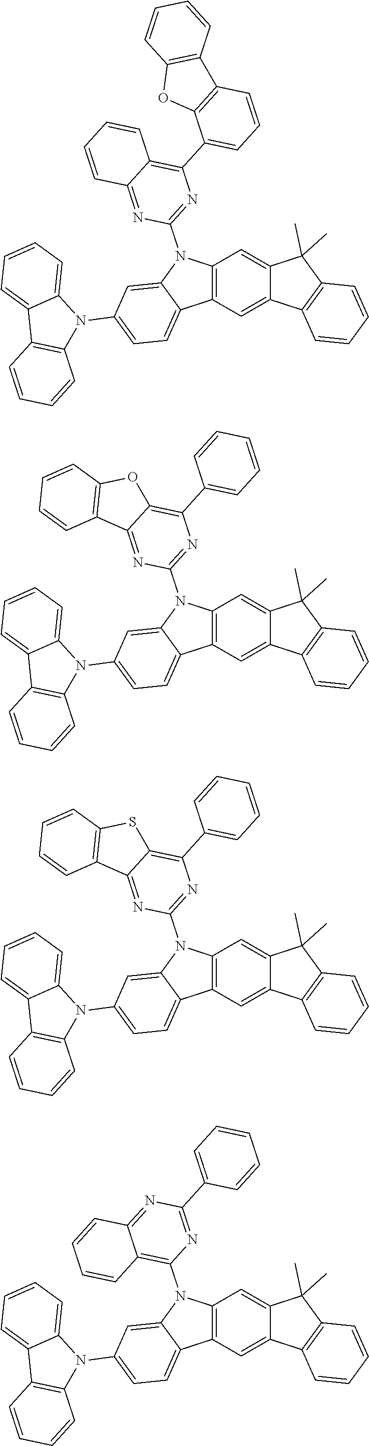 [SMILES: CC1(C)c2ccccc2-c2cc3c4ccc(-n5c6ccccc6c6ccccc65)cc4n(-c4nc(-c5cccc6c5oc5ccccc56)c5ccccc5n4)c3cc21 ; CC1(C)c2ccccc2-c2cc3c4ccc(-n5c6ccccc6c6ccccc65)cc4n(-c4nc(-c5ccccc5)c5oc6ccccc6c5n4)c3cc21 ; CC1(C)c2ccccc2-c2cc3c4ccc(-n5c6ccccc6c6ccccc65)cc4n(-c4nc(-c5ccccc5)c5sc6ccccc6c5n4)c3cc21 ; CC1(C)c2ccccc2-c2cc3c4ccc(-n5c6ccccc6c6ccccc65)cc4n(-c4nc(-c5ccccc5)nc5ccccc45)c3cc21]

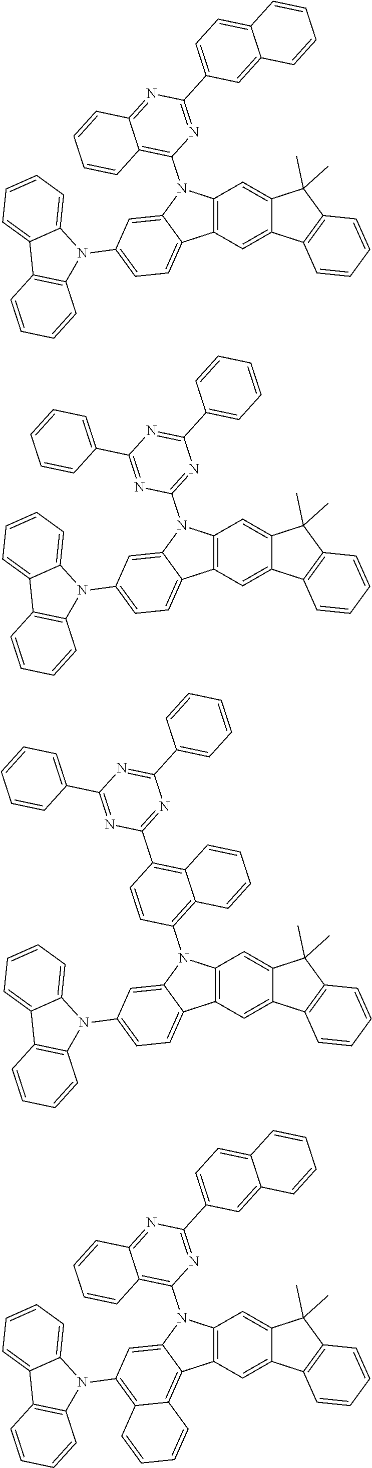 [SMILES: CC1(C)c2ccccc2-c2cc3c4ccc(-n5c6ccccc6c6ccccc65)cc4n(-c4nc(-c5ccc6ccccc6c5)nc5ccccc45)c3cc21 ; CC1(C)c2ccccc2-c2cc3c4ccc(-n5c6ccccc6c6ccccc65)cc4n(-c4nc(-c5ccccc5)nc(-c5ccccc5)n4)c3cc21 ; CC1(C)c2ccccc2-c2cc3c4ccc(-n5c6ccccc6c6ccccc65)cc4n(-c4ccc(-c5nc(-c6ccccc6)nc(-c6ccccc6)n5)c5ccccc45)c3cc21 ; CC1(C)c2ccccc2-c2cc3c4c5ccccc5c(-n5c6ccccc6c6ccccc65)cc4n(-c4nc(-c5ccc6ccccc6c5)nc5ccccc45)c3cc21]

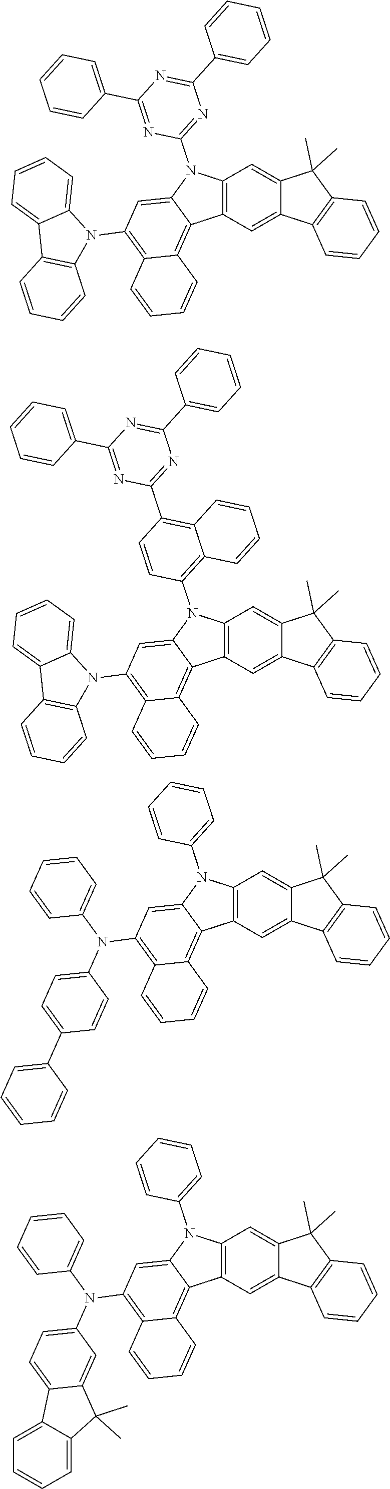

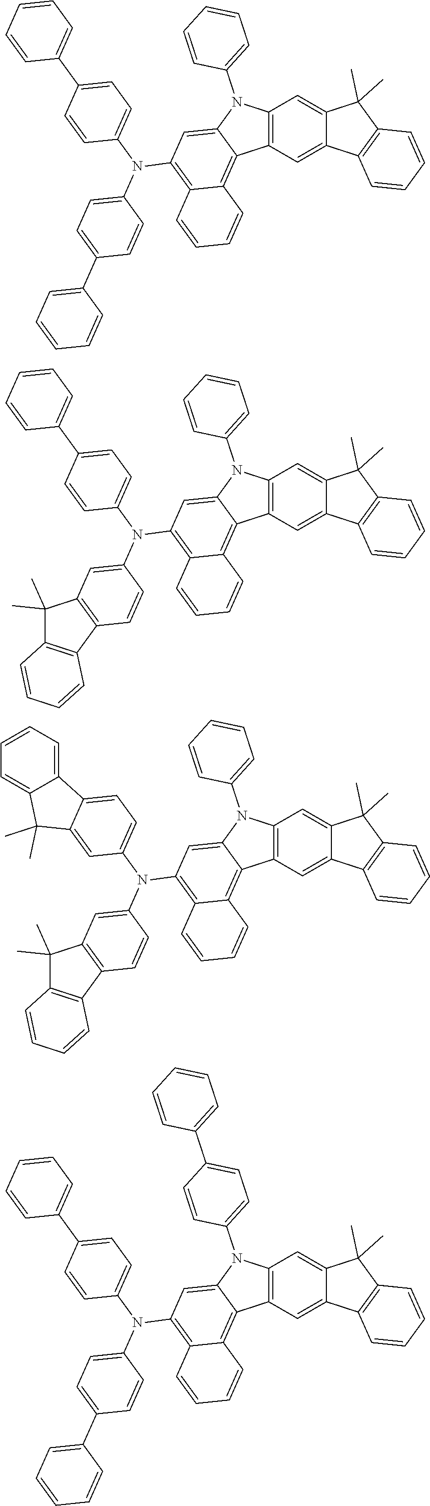

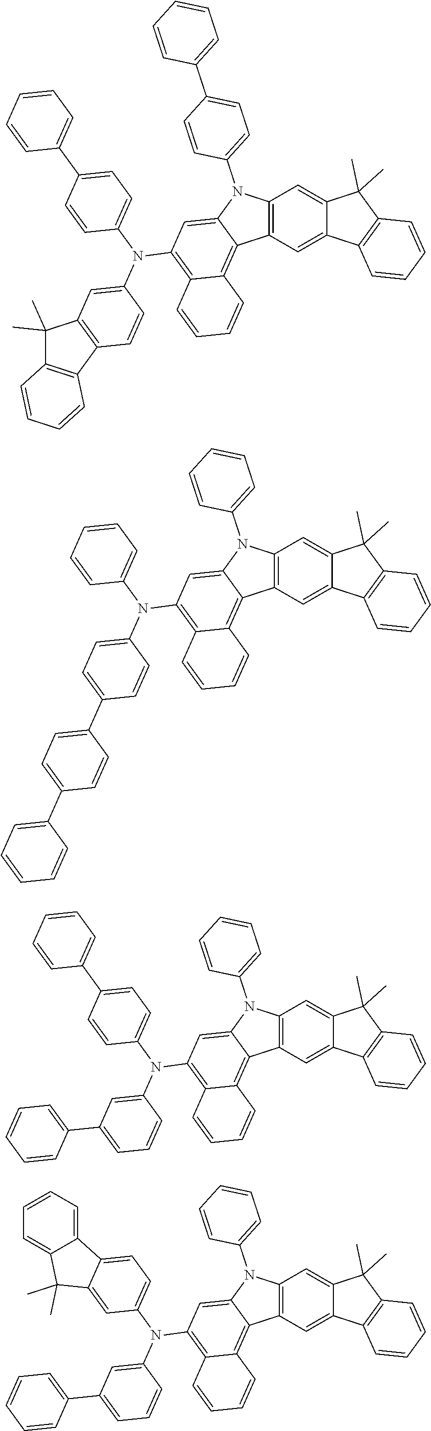

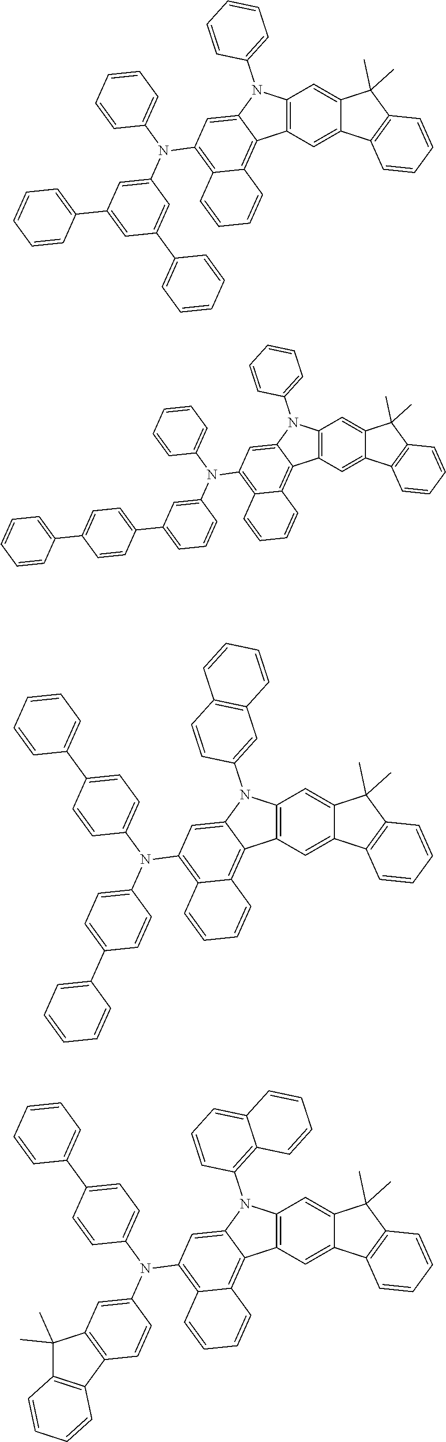 [SMILES: CC1(C)c2ccccc2-c2cc3c4c5ccccc5c(N(c5ccccc5)c5cc(-c6ccccc6)cc(-c6ccccc6)c5)cc4n(-c4ccccc4)c3cc21 ; CC1(C)c2ccccc2-c2cc3c4c5ccccc5c(N(c5ccccc5)c5cccc(-c6ccc(-c7ccccc7)cc6)c5)cc4n(-c4ccccc4)c3cc21 ; CC1(C)c2ccccc2-c2cc3c4c5ccccc5c(N(c5ccc(-c6ccccc6)cc5)c5ccc(-c6ccccc6)cc5)cc4n(-c4ccc5ccccc5c4)c3cc21 ; CC1(C)c2ccccc2-c2ccc(N(c3ccc(-c4ccccc4)cc3)c3cc4c(c5ccccc35)c3cc5c(cc3n4-c3cccc4ccccc34)C(C)(C)c3ccccc3-5)cc21]

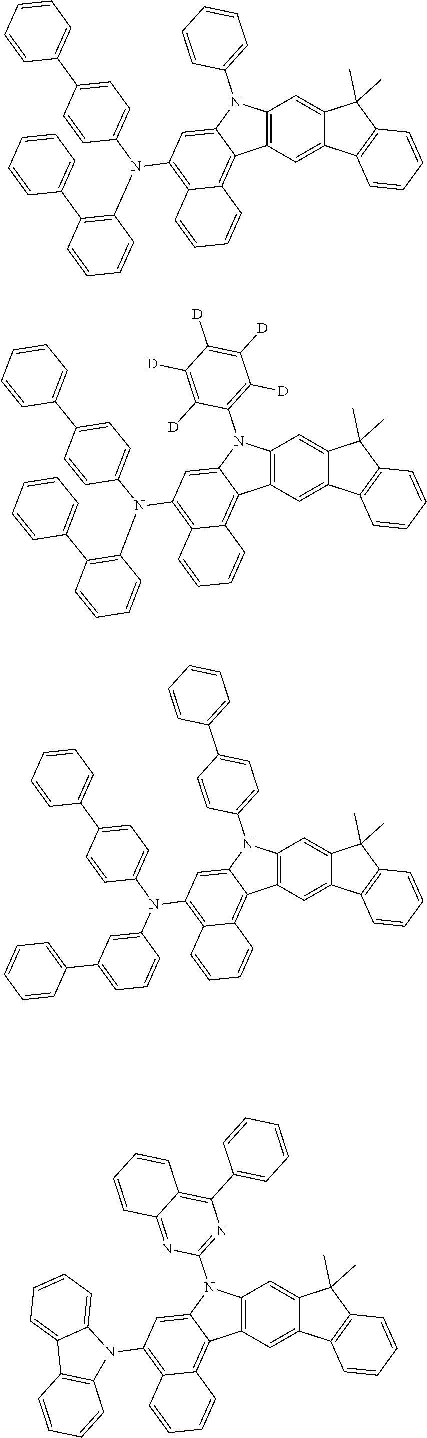 [SMILES: CC1(C)c2ccccc2-c2cc3c4c5ccccc5c(N(c5ccc(-c6ccccc6)cc5)c5ccccc5-c5ccccc5)cc4n(-c4ccccc4)c3cc21 ; [2H]c1c([2H])c([2H])c(-n2c3cc4c(cc3c3c5ccccc5c(N(c5ccc(-c6ccccc6)cc5)c5ccccc5-c5ccccc5)cc32)-c2ccccc2C4(C)C)c([2H])c1[2H] ; CC1(C)c2ccccc2-c2cc3c4c5ccccc5c(N(c5ccc(-c6ccccc6)cc5)c5cccc(-c6ccccc6)c5)cc4n(-c4ccc(-c5ccccc5)cc4)c3cc21 ; CC1(C)c2ccccc2-c2cc3c4c5ccccc5c(-n5c6ccccc6c6ccccc65)cc4n(-c4nc(-c5ccccc5)c5ccccc5n4)c3cc21]

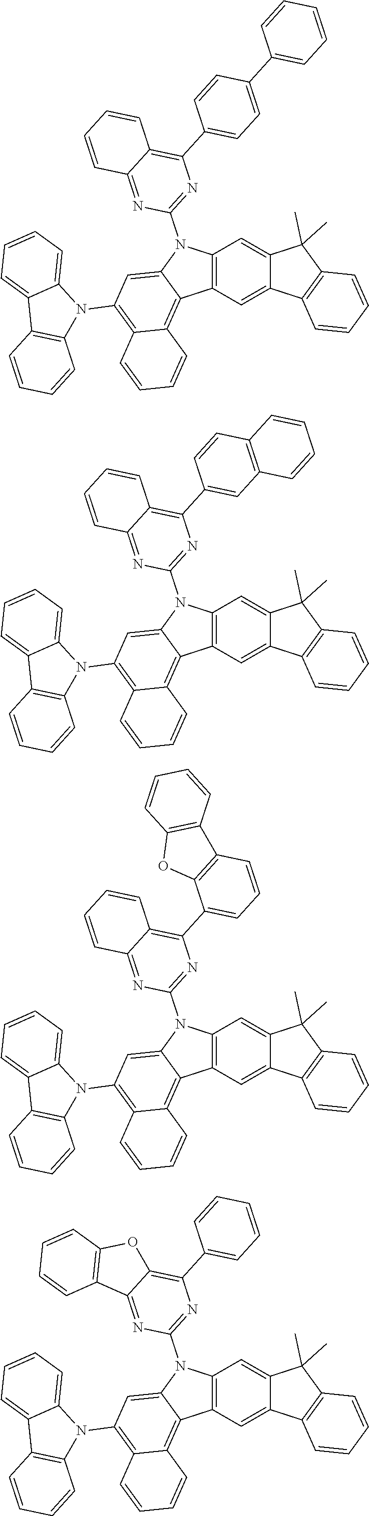

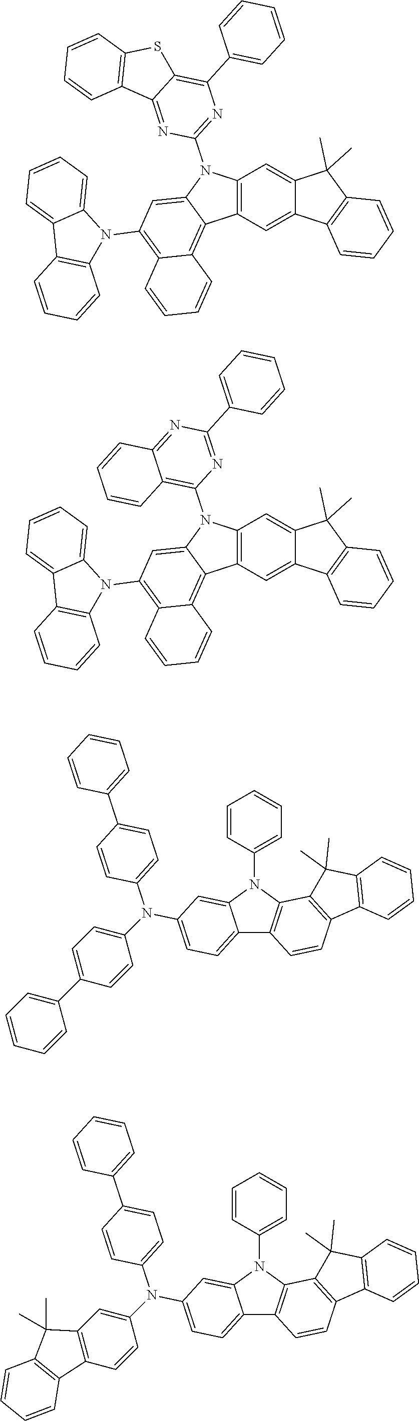 [SMILES: CC1(C)c2ccccc2-c2cc3c4c5ccccc5c(-n5c6ccccc6c6ccccc65)cc4n(-c4nc(-c5ccccc5)c5sc6ccccc6c5n4)c3cc21 ; CC1(C)c2ccccc2-c2cc3c4c5ccccc5c(-n5c6ccccc6c6ccccc65)cc4n(-c4nc(-c5ccccc5)nc5ccccc45)c3cc21 ; CC1(C)c2ccccc2-c2ccc3c4ccc(N(c5ccc(-c6ccccc6)cc5)c5ccc(-c6ccccc6)cc5)cc4n(-c4ccccc4)c3c21 ; CC1(C)c2ccccc2-c2ccc(N(c3ccc(-c4ccccc4)cc3)c3ccc4c5ccc6c(c5n(-c5ccccc5)c4c3)C(C)(C)c3ccccc3-6)cc21]

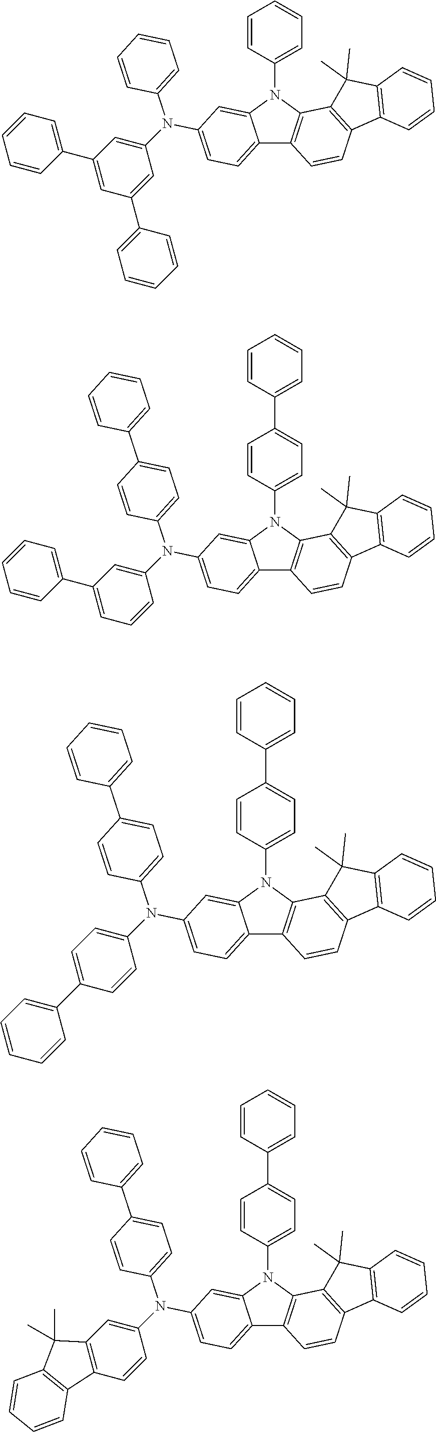 [SMILES: CC1(C)c2ccccc2-c2ccc3c4ccc(N(c5ccccc5)c5cc(-c6ccccc6)cc(-c6ccccc6)c5)cc4n(-c4ccccc4)c3c21 ; CC1(C)c2ccccc2-c2ccc3c4ccc(N(c5ccc(-c6ccccc6)cc5)c5cccc(-c6ccccc6)c5)cc4n(-c4ccc(-c5ccccc5)cc4)c3c21 ; CC1(C)c2ccccc2-c2ccc3c4ccc(N(c5ccc(-c6ccccc6)cc5)c5ccc(-c6ccccc6)cc5)cc4n(-c4ccc(-c5ccccc5)cc4)c3c21 ; CC1(C)c2ccccc2-c2ccc(N(c3ccc(-c4ccccc4)cc3)c3ccc4c5ccc6c(c5n(-c5ccc(-c7ccccc7)cc5)c4c3)C(C)(C)c3ccccc3-6)cc21]

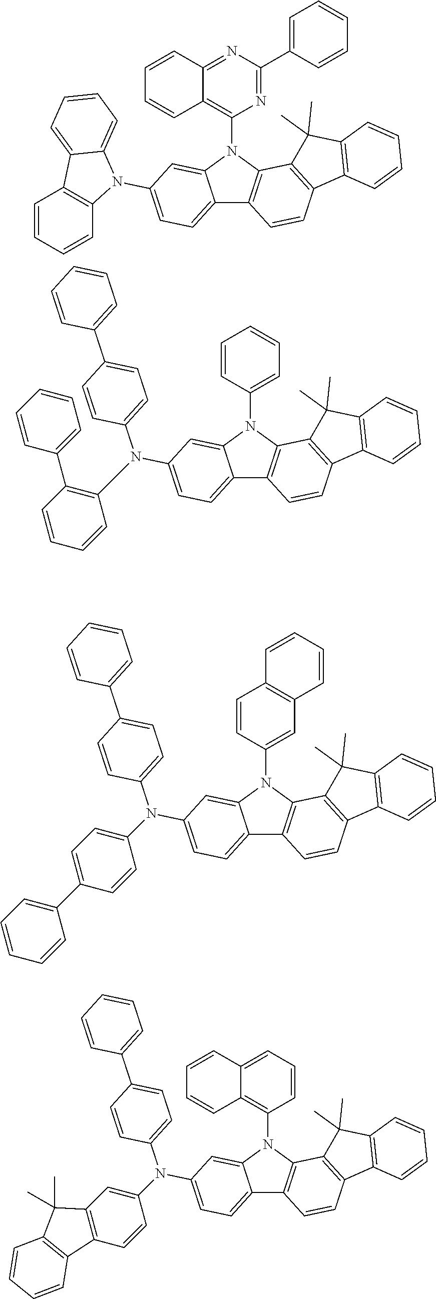

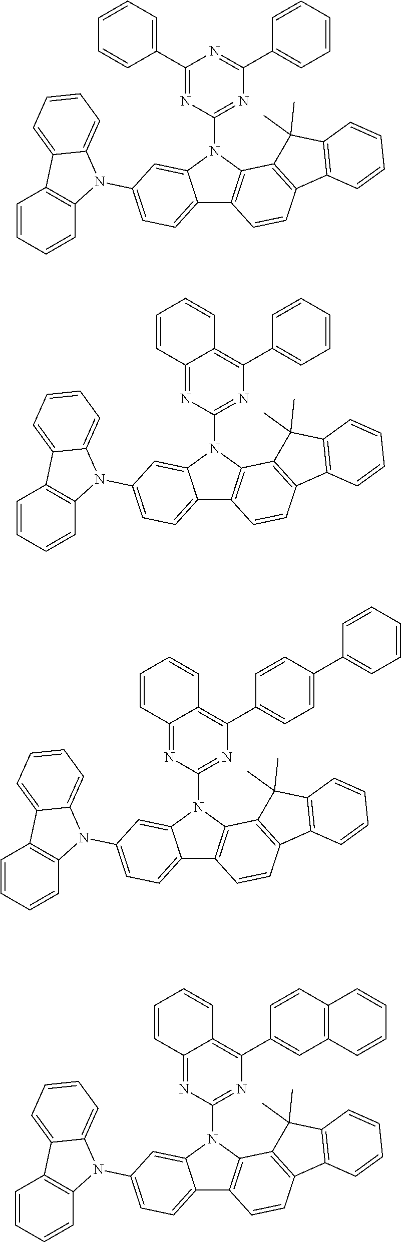 [SMILES: CC1(C)c2ccccc2-c2ccc3c4ccc(-n5c6ccccc6c6ccccc65)cc4n(-c4nc(-c5ccccc5)nc(-c5ccccc5)n4)c3c21 ; CC1(C)c2ccccc2-c2ccc3c4ccc(-n5c6ccccc6c6ccccc65)cc4n(-c4nc(-c5ccccc5)c5ccccc5n4)c3c21 ; CC1(C)c2ccccc2-c2ccc3c4ccc(-n5c6ccccc6c6ccccc65)cc4n(-c4nc(-c5ccc(-c6ccccc6)cc5)c5ccccc5n4)c3c21 ; CC1(C)c2ccccc2-c2ccc3c4ccc(-n5c6ccccc6c6ccccc65)cc4n(-c4nc(-c5ccc6ccccc6c5)c5ccccc5n4)c3c21]

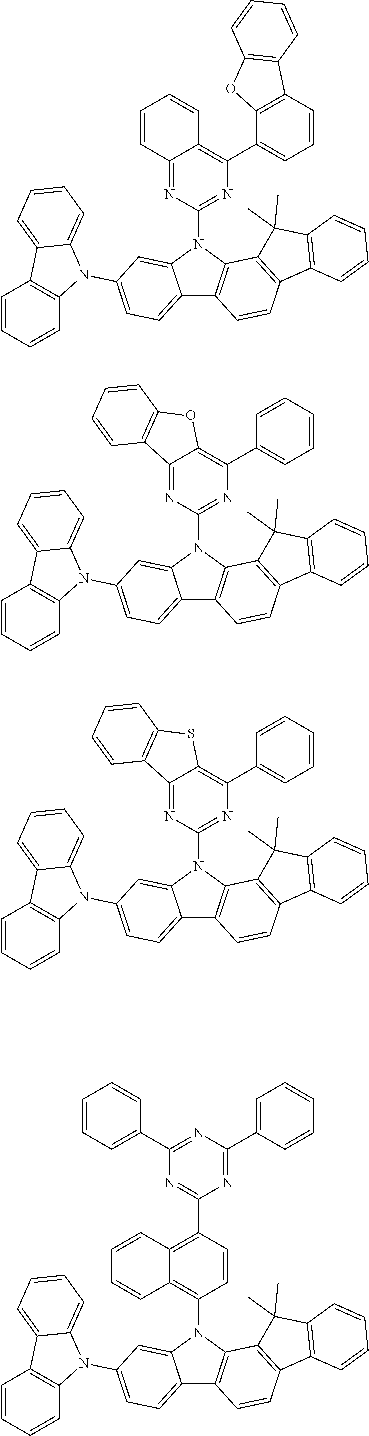 [SMILES: CC1(C)c2ccccc2-c2ccc3c4ccc(-n5c6ccccc6c6ccccc65)cc4n(-c4nc(-c5cccc6c5oc5ccccc56)c5ccccc5n4)c3c21 ; CC1(C)c2ccccc2-c2ccc3c4ccc(-n5c6ccccc6c6ccccc65)cc4n(-c4nc(-c5ccccc5)c5oc6ccccc6c5n4)c3c21 ; CC1(C)c2ccccc2-c2ccc3c4ccc(-n5c6ccccc6c6ccccc65)cc4n(-c4nc(-c5ccccc5)c5sc6ccccc6c5n4)c3c21 ; CC1(C)c2ccccc2-c2ccc3c4ccc(-n5c6ccccc6c6ccccc65)cc4n(-c4ccc(-c5nc(-c6ccccc6)nc(-c6ccccc6)n5)c5ccccc45)c3c21]

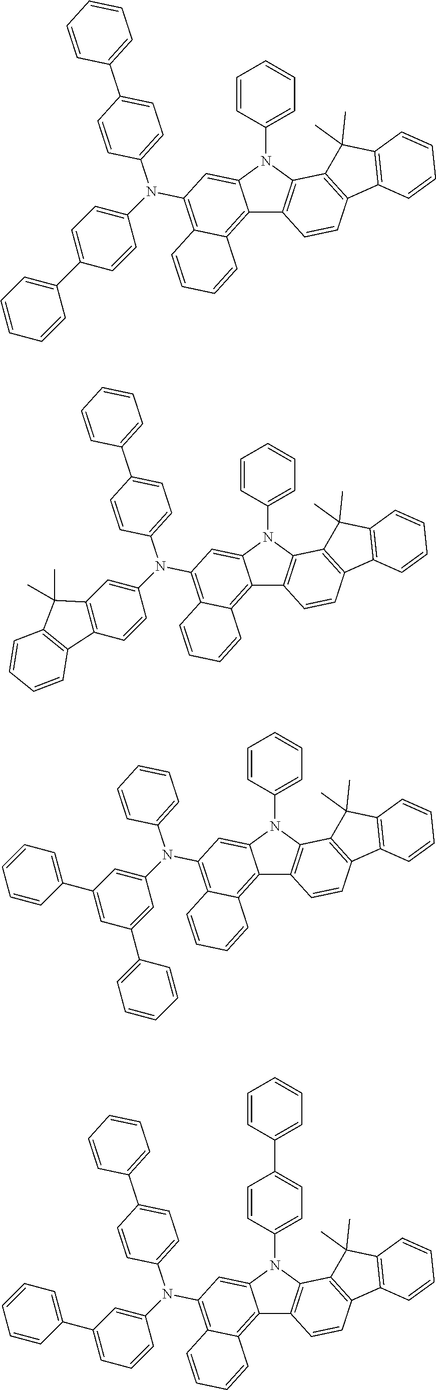 [SMILES: CC1(C)c2ccccc2-c2ccc3c4c5ccccc5c(N(c5ccc(-c6ccccc6)cc5)c5ccc(-c6ccccc6)cc5)cc4n(-c4ccccc4)c3c21 ; CC1(C)c2ccccc2-c2ccc(N(c3ccc(-c4ccccc4)cc3)c3cc4c(c5ccccc35)c3ccc5c(c3n4-c3ccccc3)C(C)(C)c3ccccc3-5)cc21 ; CC1(C)c2ccccc2-c2ccc3c4c5ccccc5c(N(c5ccccc5)c5cc(-c6ccccc6)cc(-c6ccccc6)c5)cc4n(-c4ccccc4)c3c21 ; CC1(C)c2ccccc2-c2ccc3c4c5ccccc5c(N(c5ccc(-c6ccccc6)cc5)c5cccc(-c6ccccc6)c5)cc4n(-c4ccc(-c5ccccc5)cc4)c3c21]

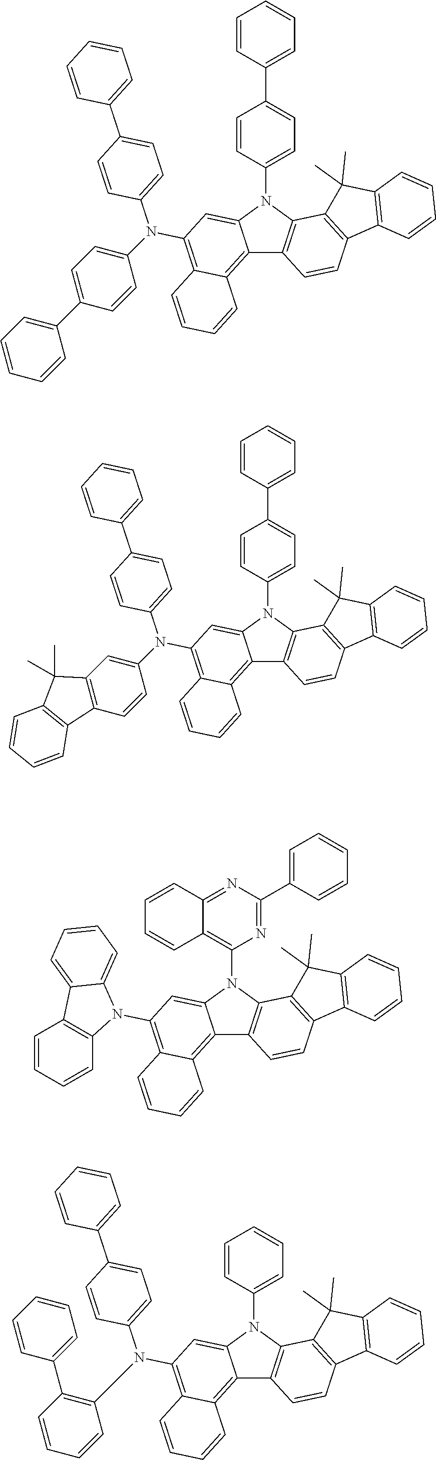 [SMILES: CC1(C)c2ccccc2-c2ccc3c4c5ccccc5c(N(c5ccc(-c6ccccc6)cc5)c5ccc(-c6ccccc6)cc5)cc4n(-c4ccc(-c5ccccc5)cc4)c3c21 ; CC1(C)c2ccccc2-c2ccc(N(c3ccc(-c4ccccc4)cc3)c3cc4c(c5ccccc35)c3ccc5c(c3n4-c3ccc(-c4ccccc4)cc3)C(C)(C)c3ccccc3-5)cc21 ; CC1(C)c2ccccc2-c2ccc3c4c5ccccc5c(-n5c6ccccc6c6ccccc65)cc4n(-c4nc(-c5ccccc5)nc5ccccc45)c3c21 ; CC1(C)c2ccccc2-c2ccc3c4c5ccccc5c(N(c5ccc(-c6ccccc6)cc5)c5ccccc5-c5ccccc5)cc4n(-c4ccccc4)c3c21]

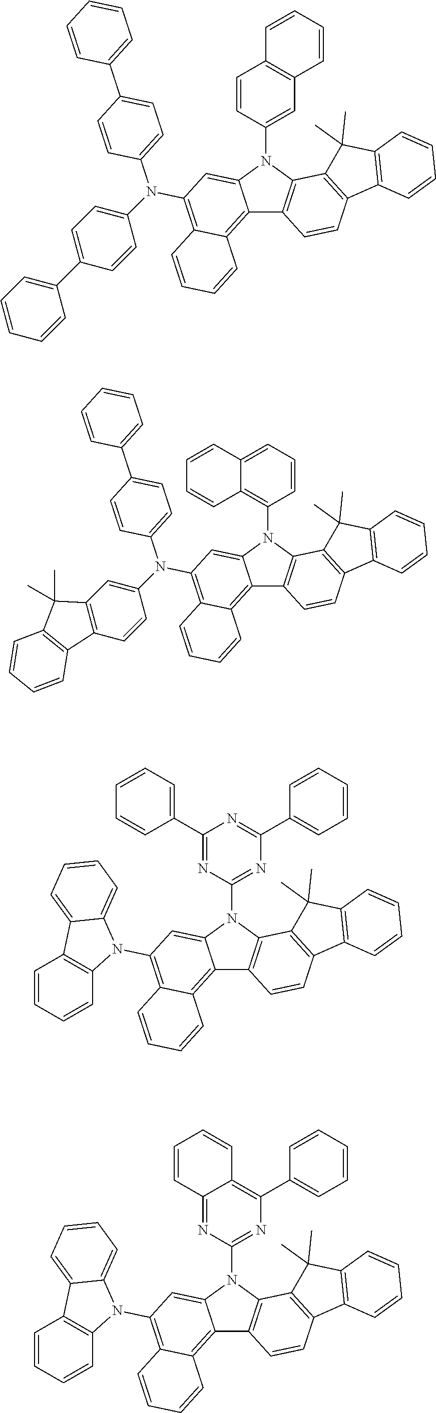 [SMILES: CC1(C)c2ccccc2-c2ccc3c4c5ccccc5c(N(c5ccc(-c6ccccc6)cc5)c5ccc(-c6ccccc6)cc5)cc4n(-c4ccc5ccccc5c4)c3c21 ; CC1(C)c2ccccc2-c2ccc(N(c3ccc(-c4ccccc4)cc3)c3cc4c(c5ccccc35)c3ccc5c(c3n4-c3cccc4ccccc34)C(C)(C)c3ccccc3-5)cc21 ; CC1(C)c2ccccc2-c2ccc3c4c5ccccc5c(-n5c6ccccc6c6ccccc65)cc4n(-c4nc(-c5ccccc5)nc(-c5ccccc5)n4)c3c21 ; CC1(C)c2ccccc2-c2ccc3c4c5ccccc5c(-n5c6ccccc6c6ccccc65)cc4n(-c4nc(-c5ccccc5)c5ccccc5n4)c3c21]

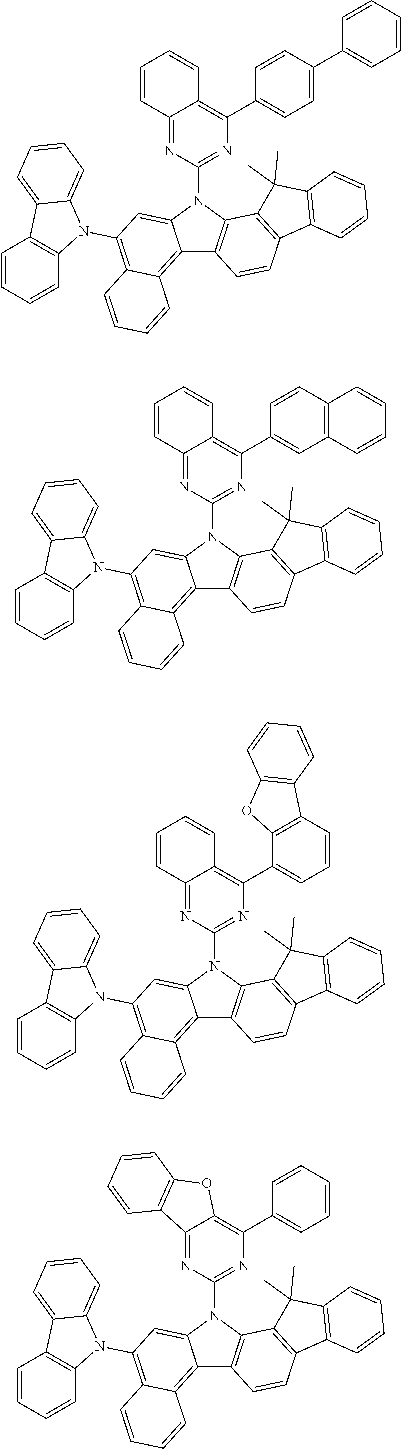 [SMILES: CC1(C)c2ccccc2-c2ccc3c4c5ccccc5c(-n5c6ccccc6c6ccccc65)cc4n(-c4nc(-c5ccc(-c6ccccc6)cc5)c5ccccc5n4)c3c21 ; CC1(C)c2ccccc2-c2ccc3c4c5ccccc5c(-n5c6ccccc6c6ccccc65)cc4n(-c4nc(-c5ccc6ccccc6c5)c5ccccc5n4)c3c21 ; CC1(C)c2ccccc2-c2ccc3c4c5ccccc5c(-n5c6ccccc6c6ccccc65)cc4n(-c4nc(-c5cccc6c5oc5ccccc56)c5ccccc5n4)c3c21 ; CC1(C)c2ccccc2-c2ccc3c4c5ccccc5c(-n5c6ccccc6c6ccccc65)cc4n(-c4nc(-c5ccccc5)c5oc6ccccc6c5n4)c3c21]

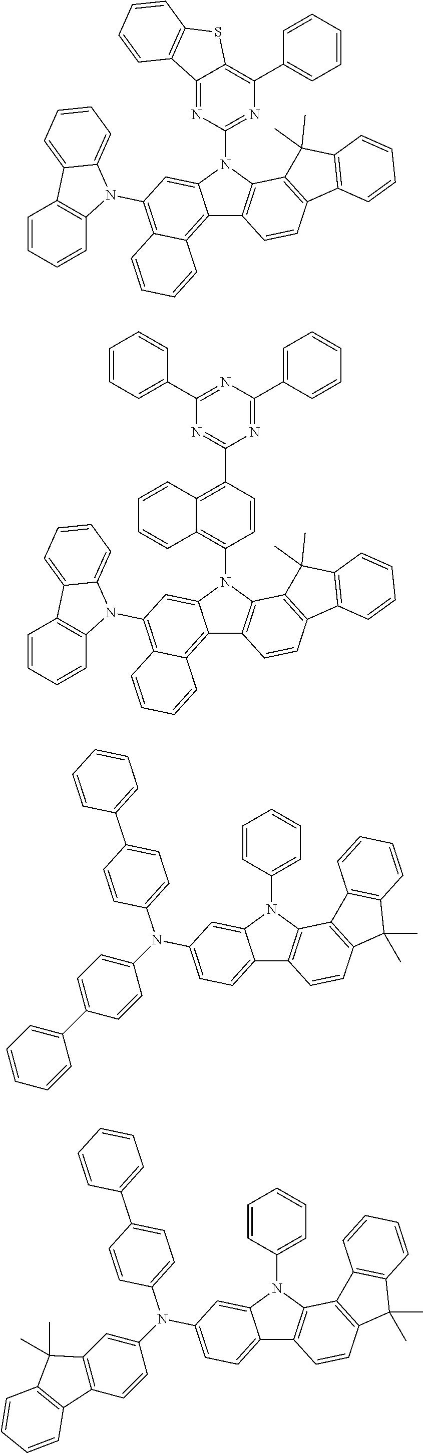

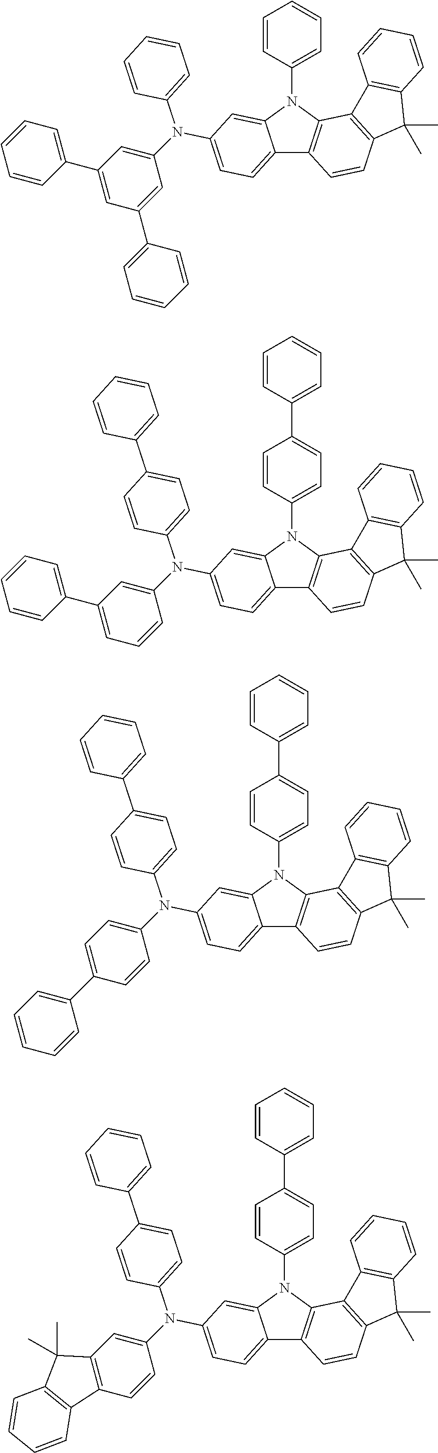 [SMILES: CC1(C)c2ccccc2-c2c1ccc1c3ccc(N(c4ccccc4)c4cc(-c5ccccc5)cc(-c5ccccc5)c4)cc3n(-c3ccccc3)c21 ; CC1(C)c2ccccc2-c2c1ccc1c3ccc(N(c4ccc(-c5ccccc5)cc4)c4cccc(-c5ccccc5)c4)cc3n(-c3ccc(-c4ccccc4)cc3)c21 ; CC1(C)c2ccccc2-c2c1ccc1c3ccc(N(c4ccc(-c5ccccc5)cc4)c4ccc(-c5ccccc5)cc4)cc3n(-c3ccc(-c4ccccc4)cc3)c21 ; CC1(C)c2ccccc2-c2ccc(N(c3ccc(-c4ccccc4)cc3)c3ccc4c5ccc6c(c5n(-c5ccc(-c7ccccc7)cc5)c4c3)-c3ccccc3C6(C)C)cc21]

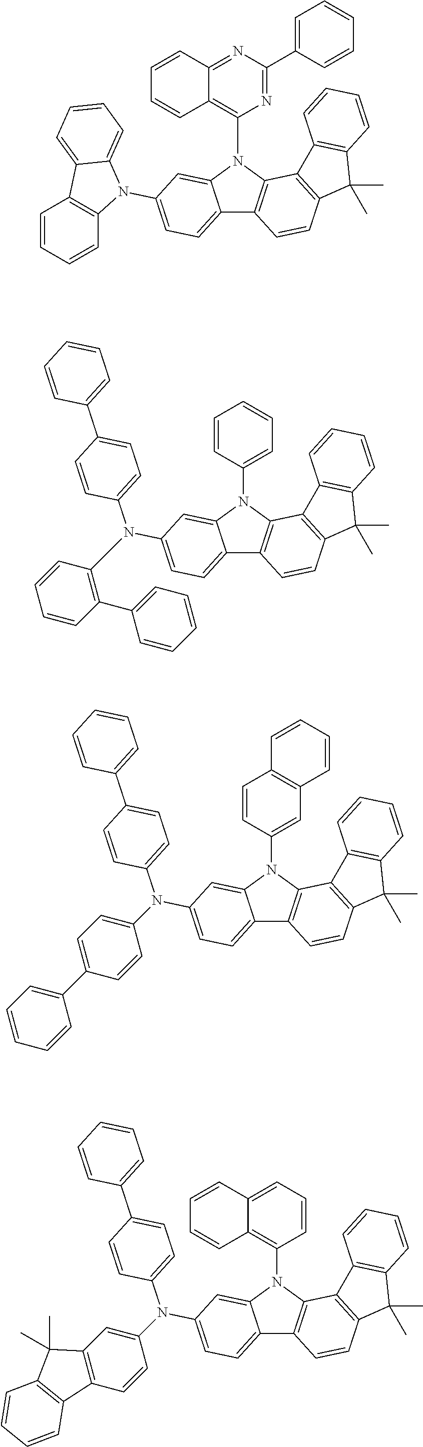 [SMILES: CC1(C)c2ccccc2-c2c1ccc1c3ccc(-n4c5ccccc5c5ccccc54)cc3n(-c3nc(-c4ccccc4)nc4ccccc34)c21 ; CC1(C)c2ccccc2-c2c1ccc1c3ccc(N(c4ccc(-c5ccccc5)cc4)c4ccccc4-c4ccccc4)cc3n(-c3ccccc3)c21 ; CC1(C)c2ccccc2-c2c1ccc1c3ccc(N(c4ccc(-c5ccccc5)cc4)c4ccc(-c5ccccc5)cc4)cc3n(-c3ccc4ccccc4c3)c21 ; CC1(C)c2ccccc2-c2ccc(N(c3ccc(-c4ccccc4)cc3)c3ccc4c5ccc6c(c5n(-c5cccc7ccccc57)c4c3)-c3ccccc3C6(C)C)cc21]

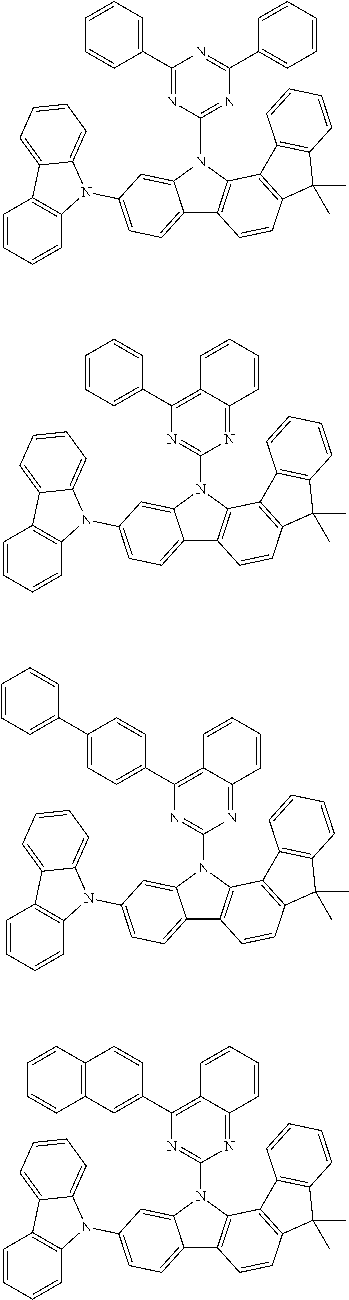 [SMILES: CC1(C)c2ccccc2-c2c1ccc1c3ccc(-n4c5ccccc5c5ccccc54)cc3n(-c3nc(-c4ccccc4)nc(-c4ccccc4)n3)c21 ; CC1(C)c2ccccc2-c2c1ccc1c3ccc(-n4c5ccccc5c5ccccc54)cc3n(-c3nc(-c4ccccc4)c4ccccc4n3)c21 ; CC1(C)c2ccccc2-c2c1ccc1c3ccc(-n4c5ccccc5c5ccccc54)cc3n(-c3nc(-c4ccc(-c5ccccc5)cc4)c4ccccc4n3)c21 ; CC1(C)c2ccccc2-c2c1ccc1c3ccc(-n4c5ccccc5c5ccccc54)cc3n(-c3nc(-c4ccc5ccccc5c4)c4ccccc4n3)c21]

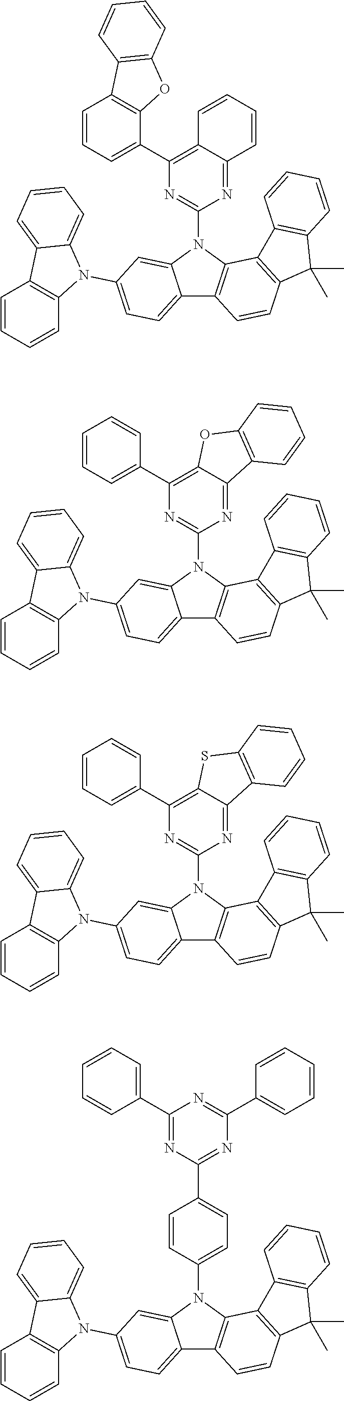 [SMILES: CC1(C)c2ccccc2-c2c1ccc1c3ccc(-n4c5ccccc5c5ccccc54)cc3n(-c3nc(-c4cccc5c4oc4ccccc45)c4ccccc4n3)c21 ; CC1(C)c2ccccc2-c2c1ccc1c3ccc(-n4c5ccccc5c5ccccc54)cc3n(-c3nc(-c4ccccc4)c4oc5ccccc5c4n3)c21 ; CC1(C)c2ccccc2-c2c1ccc1c3ccc(-n4c5ccccc5c5ccccc54)cc3n(-c3nc(-c4ccccc4)c4sc5ccccc5c4n3)c21 ; CC1(C)c2ccccc2-c2c1ccc1c3ccc(-n4c5ccccc5c5ccccc54)cc3n(-c3ccc(-c4nc(-c5ccccc5)nc(-c5ccccc5)n4)cc3)c21]

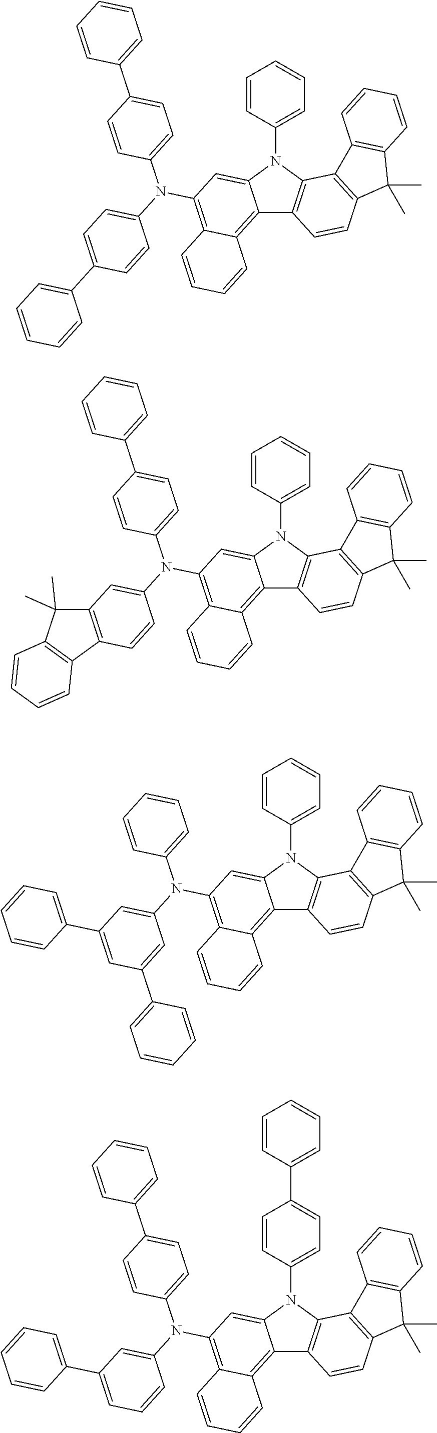 [SMILES: CC1(C)c2ccccc2-c2c1ccc1c3c4ccccc4c(N(c4ccc(-c5ccccc5)cc4)c4ccc(-c5ccccc5)cc4)cc3n(-c3ccccc3)c21 ; CC1(C)c2ccccc2-c2ccc(N(c3ccc(-c4ccccc4)cc3)c3cc4c(c5ccccc35)c3ccc5c(c3n4-c3ccccc3)-c3ccccc3C5(C)C)cc21 ; CC1(C)c2ccccc2-c2c1ccc1c3c4ccccc4c(N(c4ccccc4)c4cc(-c5ccccc5)cc(-c5ccccc5)c4)cc3n(-c3ccccc3)c21 ; CC1(C)c2ccccc2-c2c1ccc1c3c4ccccc4c(N(c4ccc(-c5ccccc5)cc4)c4cccc(-c5ccccc5)c4)cc3n(-c3ccc(-c4ccccc4)cc3)c21]

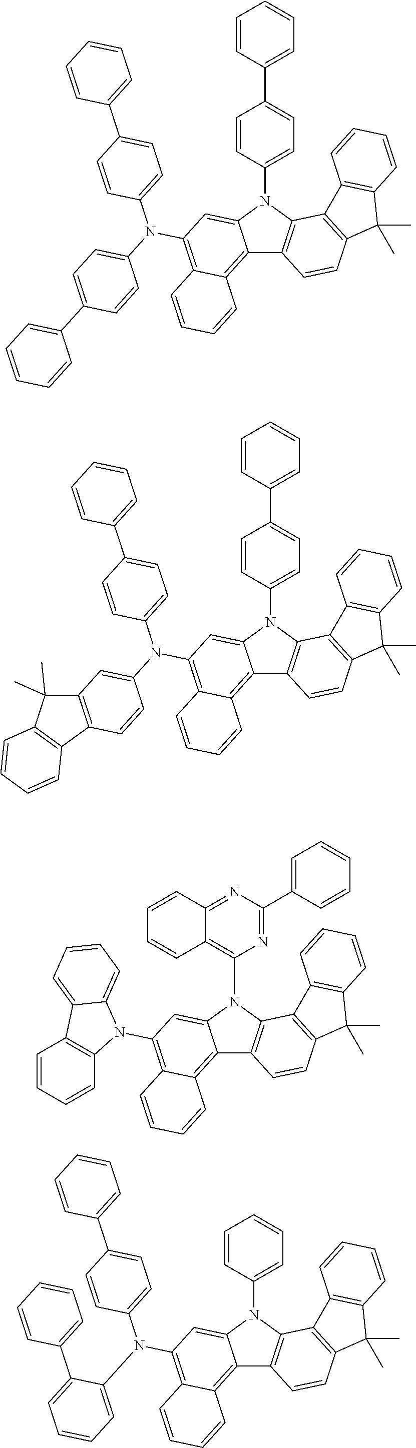 [SMILES: CC1(C)c2ccccc2-c2c1ccc1c3c4ccccc4c(N(c4ccc(-c5ccccc5)cc4)c4ccc(-c5ccccc5)cc4)cc3n(-c3ccc(-c4ccccc4)cc3)c21 ; CC1(C)c2ccccc2-c2ccc(N(c3ccc(-c4ccccc4)cc3)c3cc4c(c5ccccc35)c3ccc5c(c3n4-c3ccc(-c4ccccc4)cc3)-c3ccccc3C5(C)C)cc21 ; CC1(C)c2ccccc2-c2c1ccc1c3c4ccccc4c(-n4c5ccccc5c5ccccc54)cc3n(-c3nc(-c4ccccc4)nc4ccccc34)c21 ; CC1(C)c2ccccc2-c2c1ccc1c3c4ccccc4c(N(c4ccc(-c5ccccc5)cc4)c4ccccc4-c4ccccc4)cc3n(-c3ccccc3)c21]

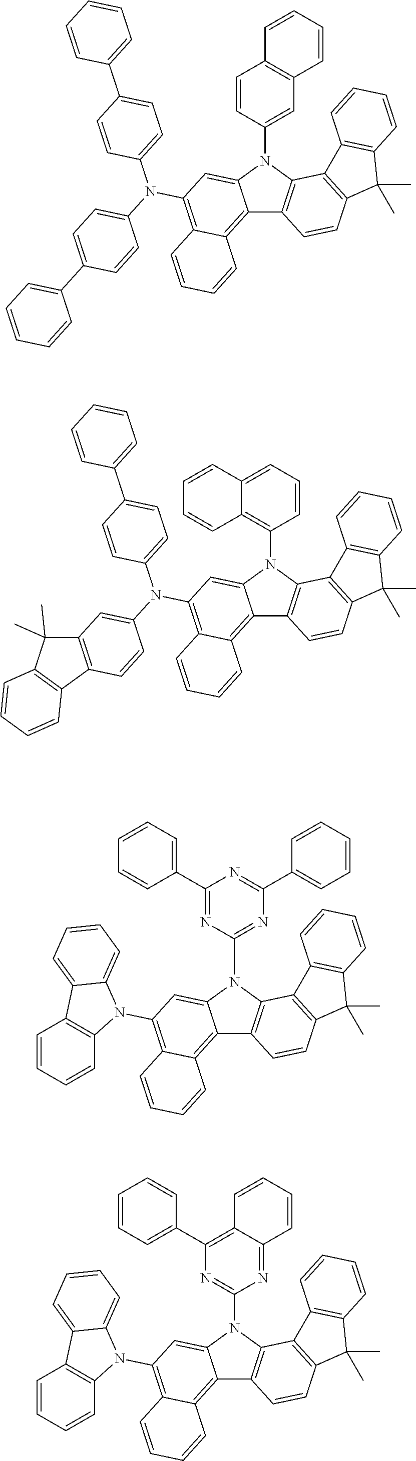 [SMILES: CC1(C)c2ccccc2-c2c1ccc1c3c4ccccc4c(N(c4ccc(-c5ccccc5)cc4)c4ccc(-c5ccccc5)cc4)cc3n(-c3ccc4ccccc4c3)c21 ; CC1(C)c2ccccc2-c2ccc(N(c3ccc(-c4ccccc4)cc3)c3cc4c(c5ccccc35)c3ccc5c(c3n4-c3cccc4ccccc34)-c3ccccc3C5(C)C)cc21 ; CC1(C)c2ccccc2-c2c1ccc1c3c4ccccc4c(-n4c5ccccc5c5ccccc54)cc3n(-c3nc(-c4ccccc4)nc(-c4ccccc4)n3)c21 ; CC1(C)c2ccccc2-c2c1ccc1c3c4ccccc4c(-n4c5ccccc5c5ccccc54)cc3n(-c3nc(-c4ccccc4)c4ccccc4n3)c21]

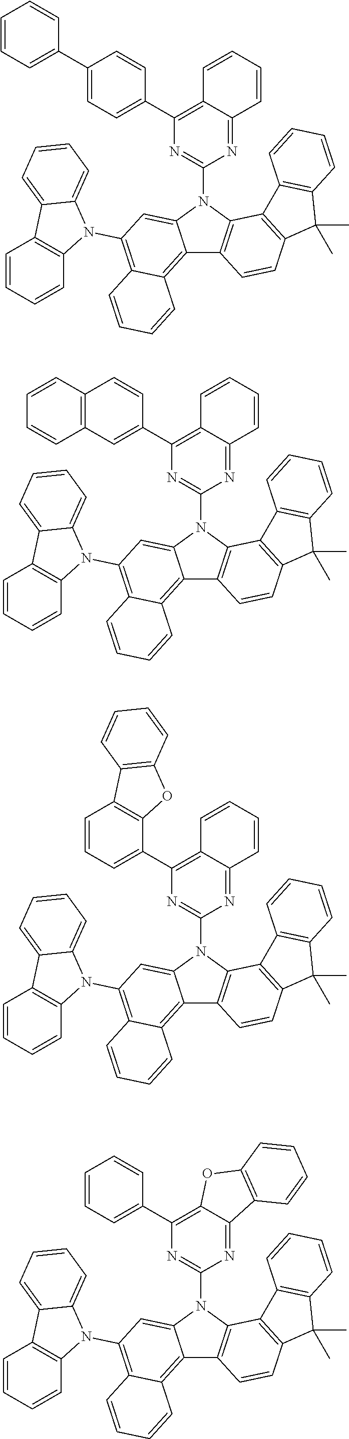 [SMILES: CC1(C)c2ccccc2-c2c1ccc1c3c4ccccc4c(-n4c5ccccc5c5ccccc54)cc3n(-c3nc(-c4ccc(-c5ccccc5)cc4)c4ccccc4n3)c21 ; CC1(C)c2ccccc2-c2c1ccc1c3c4ccccc4c(-n4c5ccccc5c5ccccc54)cc3n(-c3nc(-c4ccc5ccccc5c4)c4ccccc4n3)c21 ; CC1(C)c2ccccc2-c2c1ccc1c3c4ccccc4c(-n4c5ccccc5c5ccccc54)cc3n(-c3nc(-c4cccc5c4oc4ccccc45)c4ccccc4n3)c21 ; CC1(C)c2ccccc2-c2c1ccc1c3c4ccccc4c(-n4c5ccccc5c5ccccc54)cc3n(-c3nc(-c4ccccc4)c4oc5ccccc5c4n3)c21]

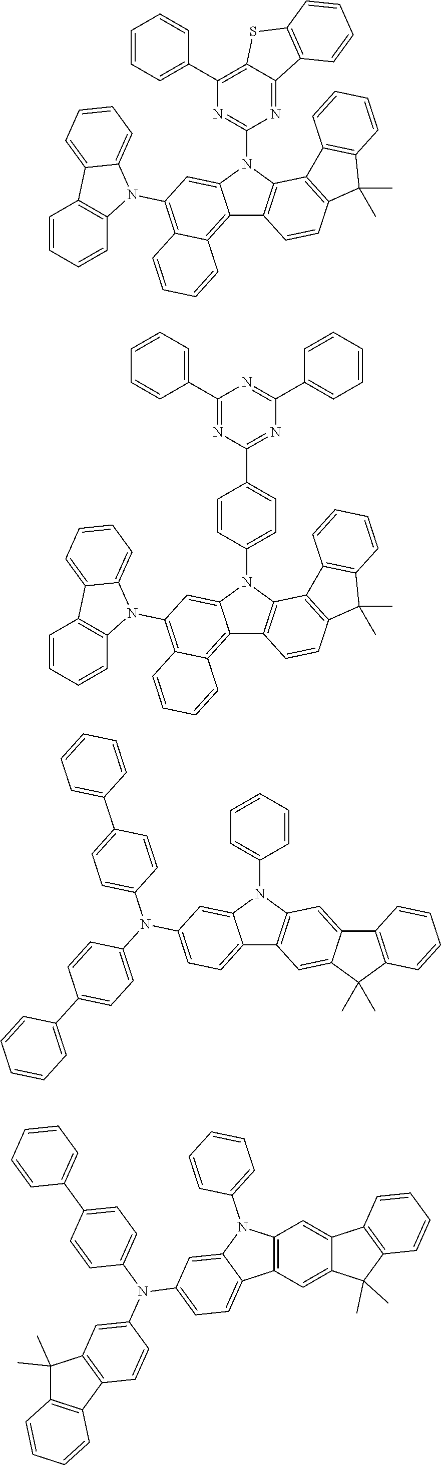

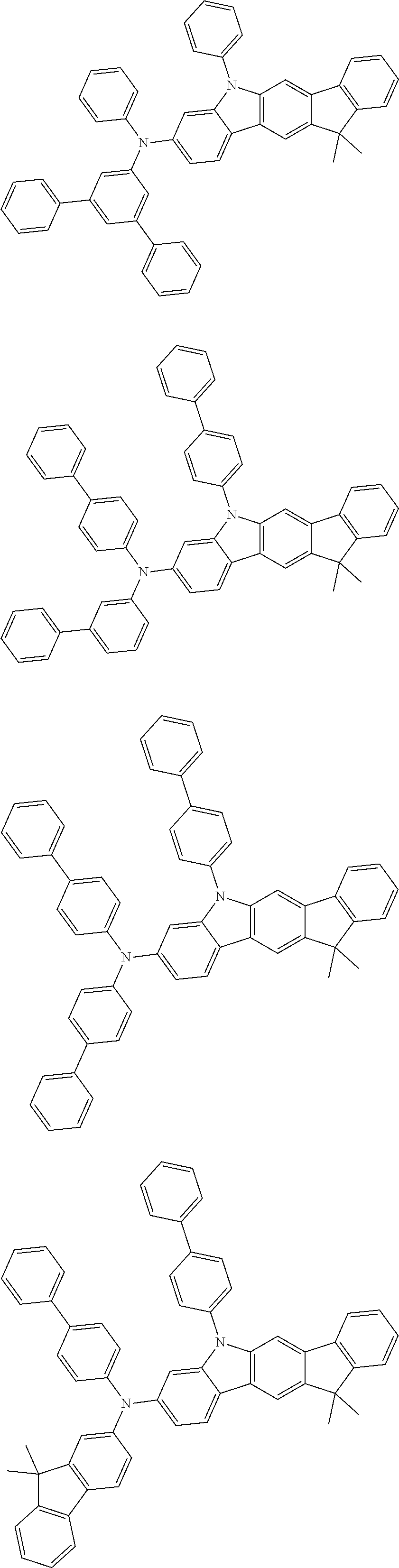 [SMILES: CC1(C)c2ccccc2-c2cc3c(cc21)c1ccc(N(c2ccccc2)c2cc(-c4ccccc4)cc(-c4ccccc4)c2)cc1n3-c1ccccc1 ; CC1(C)c2ccccc2-c2cc3c(cc21)c1ccc(N(c2ccc(-c4ccccc4)cc2)c2cccc(-c4ccccc4)c2)cc1n3-c1ccc(-c2ccccc2)cc1 ; CC1(C)c2ccccc2-c2cc3c(cc21)c1ccc(N(c2ccc(-c4ccccc4)cc2)c2ccc(-c4ccccc4)cc2)cc1n3-c1ccc(-c2ccccc2)cc1 ; CC1(C)c2ccccc2-c2ccc(N(c3ccc(-c4ccccc4)cc3)c3ccc4c5cc6c(cc5n(-c5ccc(-c7ccccc7)cc5)c4c3)-c3ccccc3C6(C)C)cc21]

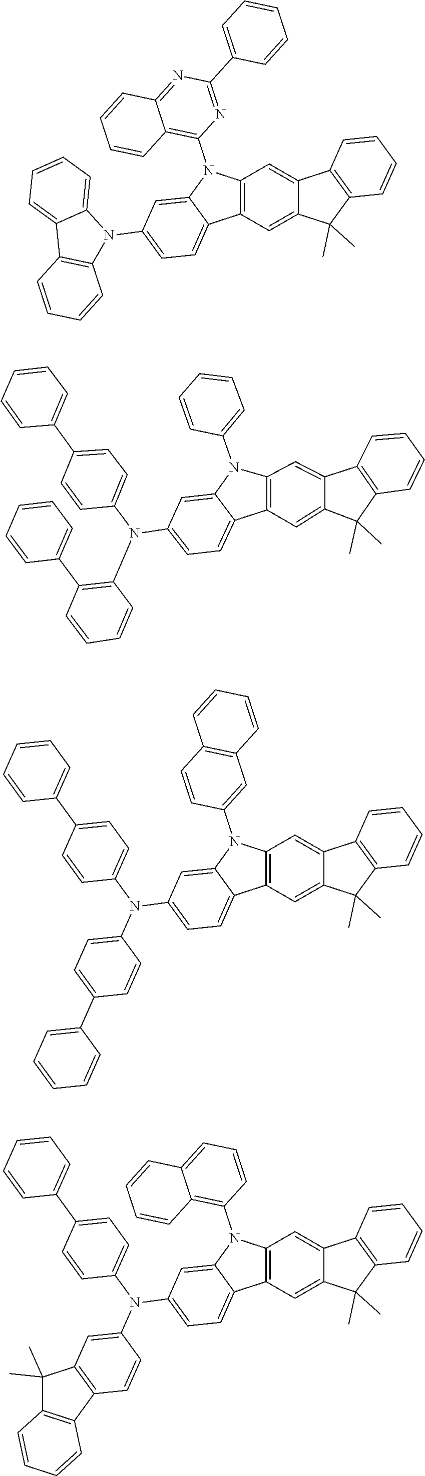 [SMILES: CC1(C)c2ccccc2-c2cc3c(cc21)c1ccc(-n2c4ccccc4c4ccccc42)cc1n3-c1nc(-c2ccccc2)nc2ccccc12 ; CC1(C)c2ccccc2-c2cc3c(cc21)c1ccc(N(c2ccc(-c4ccccc4)cc2)c2ccccc2-c2ccccc2)cc1n3-c1ccccc1 ; CC1(C)c2ccccc2-c2cc3c(cc21)c1ccc(N(c2ccc(-c4ccccc4)cc2)c2ccc(-c4ccccc4)cc2)cc1n3-c1ccc2ccccc2c1 ; CC1(C)c2ccccc2-c2ccc(N(c3ccc(-c4ccccc4)cc3)c3ccc4c5cc6c(cc5n(-c5cccc7ccccc57)c4c3)-c3ccccc3C6(C)C)cc21]

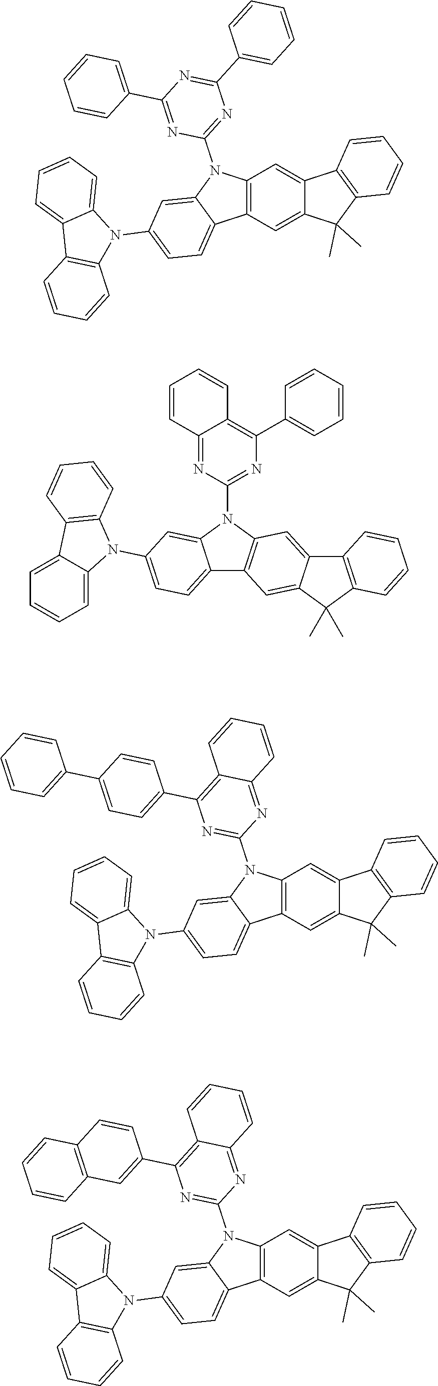 [SMILES: CC1(C)c2ccccc2-c2cc3c(cc21)c1ccc(-n2c4ccccc4c4ccccc42)cc1n3-c1nc(-c2ccccc2)nc(-c2ccccc2)n1 ; CC1(C)c2ccccc2-c2cc3c(cc21)c1ccc(-n2c4ccccc4c4ccccc42)cc1n3-c1nc(-c2ccccc2)c2ccccc2n1 ; CC1(C)c2ccccc2-c2cc3c(cc21)c1ccc(-n2c4ccccc4c4ccccc42)cc1n3-c1nc(-c2ccc(-c3ccccc3)cc2)c2ccccc2n1 ; CC1(C)c2ccccc2-c2cc3c(cc21)c1ccc(-n2c4ccccc4c4ccccc42)cc1n3-c1nc(-c2ccc3ccccc3c2)c2ccccc2n1]

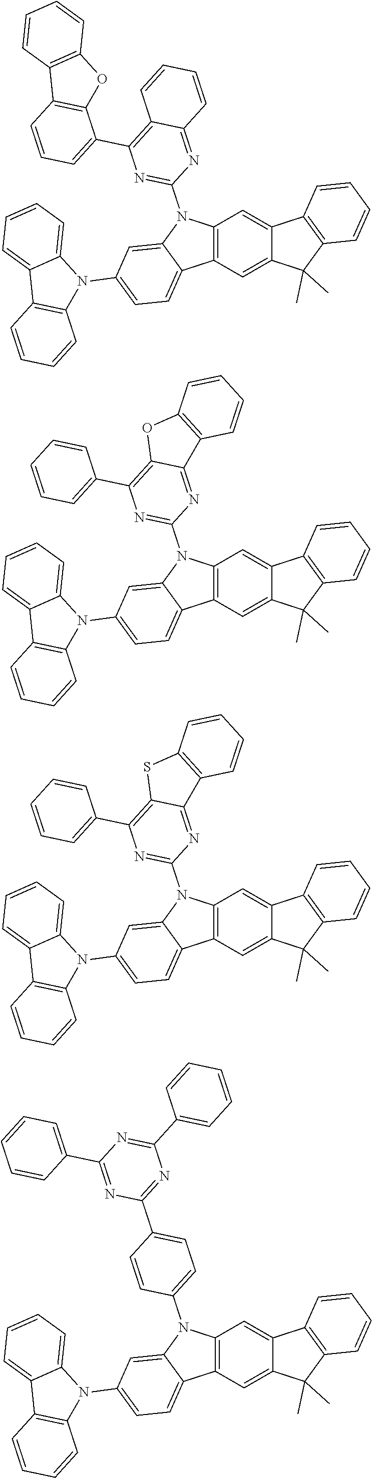 [SMILES: CC1(C)c2ccccc2-c2cc3c(cc21)c1ccc(-n2c4ccccc4c4ccccc42)cc1n3-c1nc(-c2cccc3c2oc2ccccc23)c2ccccc2n1 ; CC1(C)c2ccccc2-c2cc3c(cc21)c1ccc(-n2c4ccccc4c4ccccc42)cc1n3-c1nc(-c2ccccc2)c2oc3ccccc3c2n1 ; CC1(C)c2ccccc2-c2cc3c(cc21)c1ccc(-n2c4ccccc4c4ccccc42)cc1n3-c1nc(-c2ccccc2)c2sc3ccccc3c2n1 ; CC1(C)c2ccccc2-c2cc3c(cc21)c1ccc(-n2c4ccccc4c4ccccc42)cc1n3-c1ccc(-c2nc(-c3ccccc3)nc(-c3ccccc3)n2)cc1]

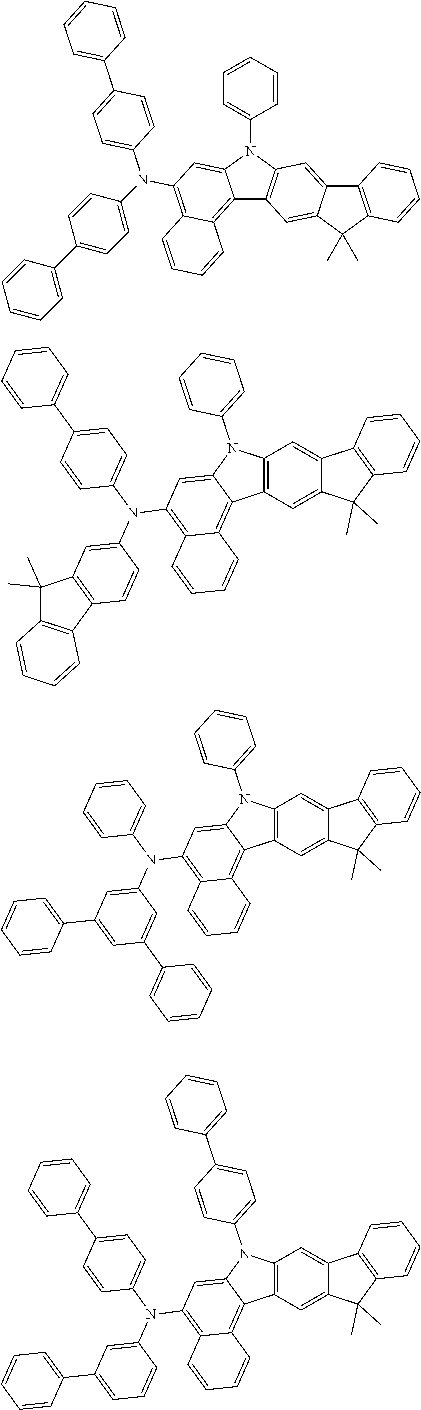

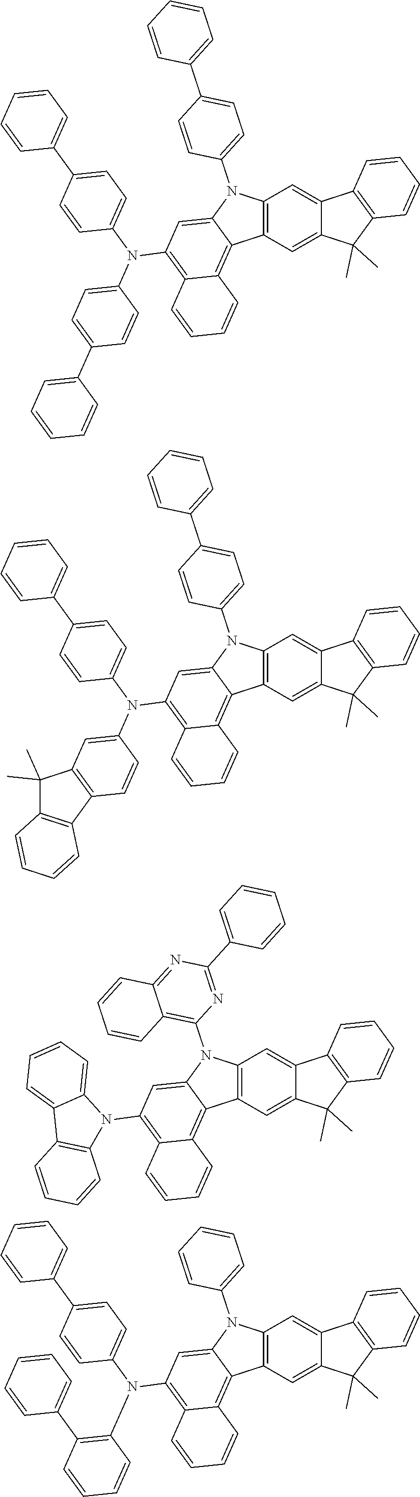

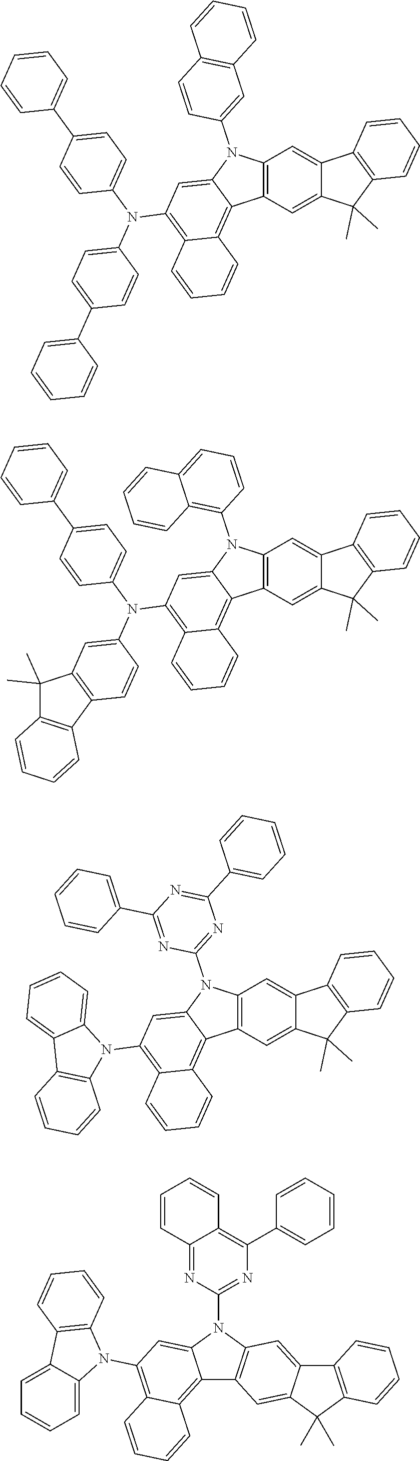

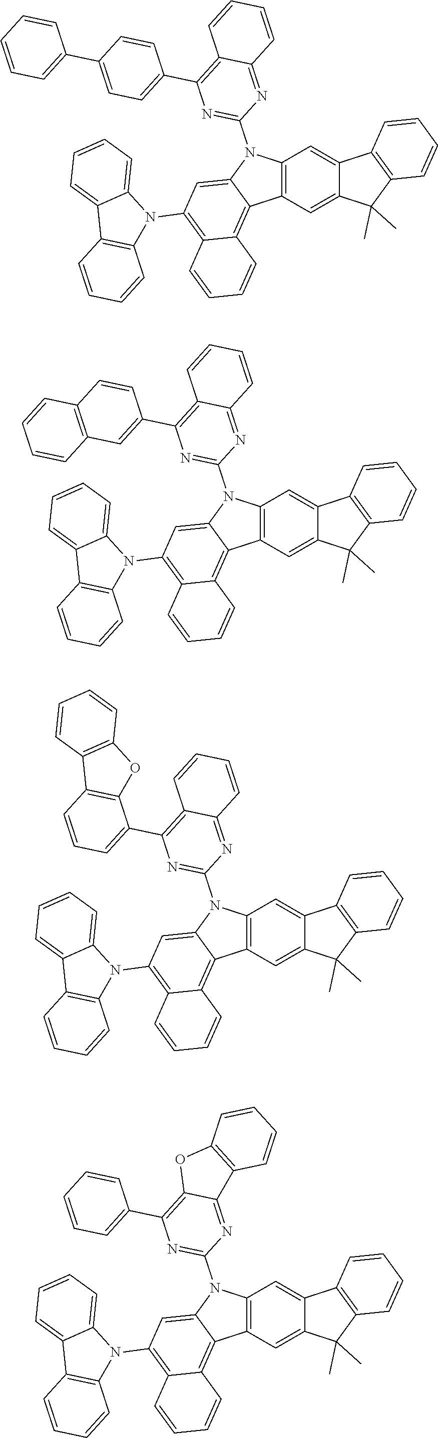

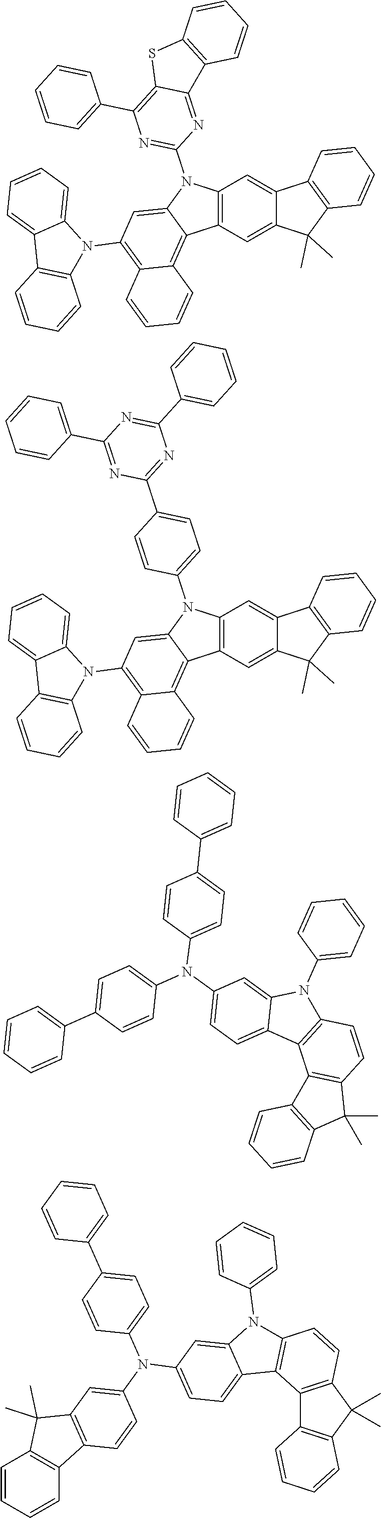

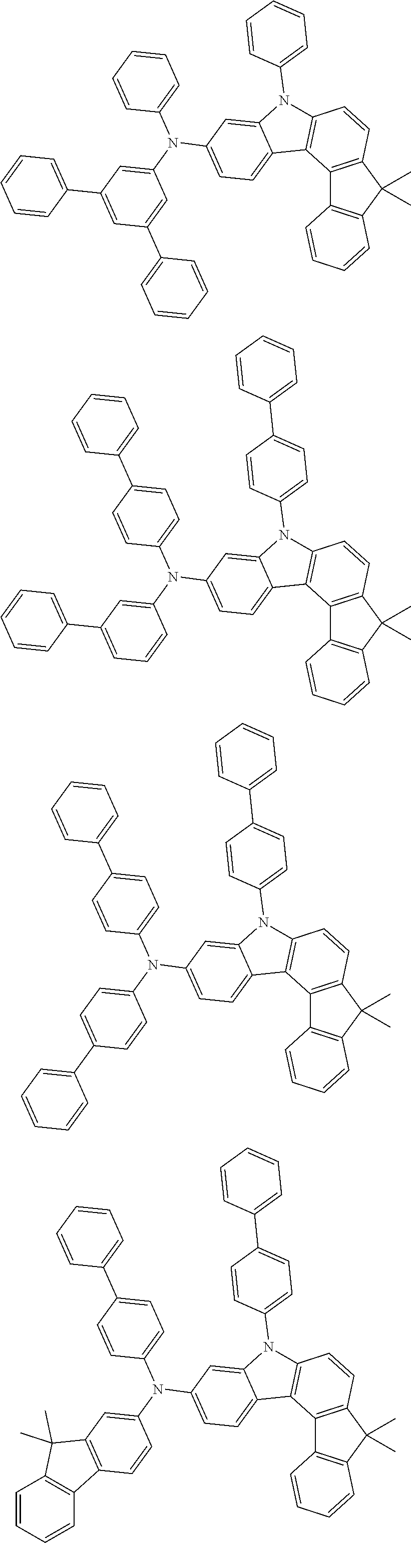 [SMILES: CC1(C)c2ccccc2-c2c1ccc1c2c2ccc(N(c3ccccc3)c3cc(-c4ccccc4)cc(-c4ccccc4)c3)cc2n1-c1ccccc1 ; CC1(C)c2ccccc2-c2c1ccc1c2c2ccc(N(c3ccc(-c4ccccc4)cc3)c3cccc(-c4ccccc4)c3)cc2n1-c1ccc(-c2ccccc2)cc1 ; CC1(C)c2ccccc2-c2c1ccc1c2c2ccc(N(c3ccc(-c4ccccc4)cc3)c3ccc(-c4ccccc4)cc3)cc2n1-c1ccc(-c2ccccc2)cc1 ; CC1(C)c2ccccc2-c2ccc(N(c3ccc(-c4ccccc4)cc3)c3ccc4c5c6c(ccc5n(-c5ccc(-c7ccccc7)cc5)c4c3)C(C)(C)c3ccccc3-6)cc21]

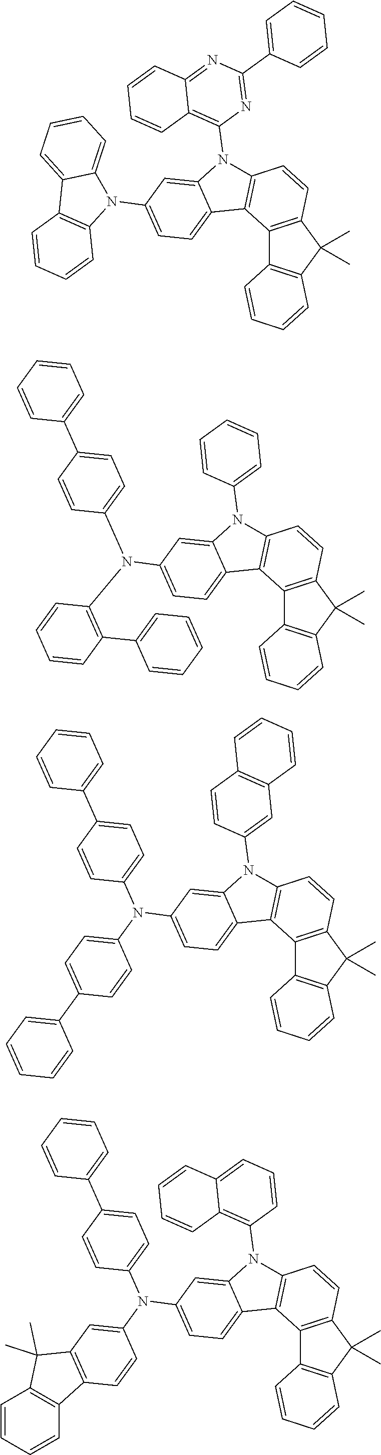

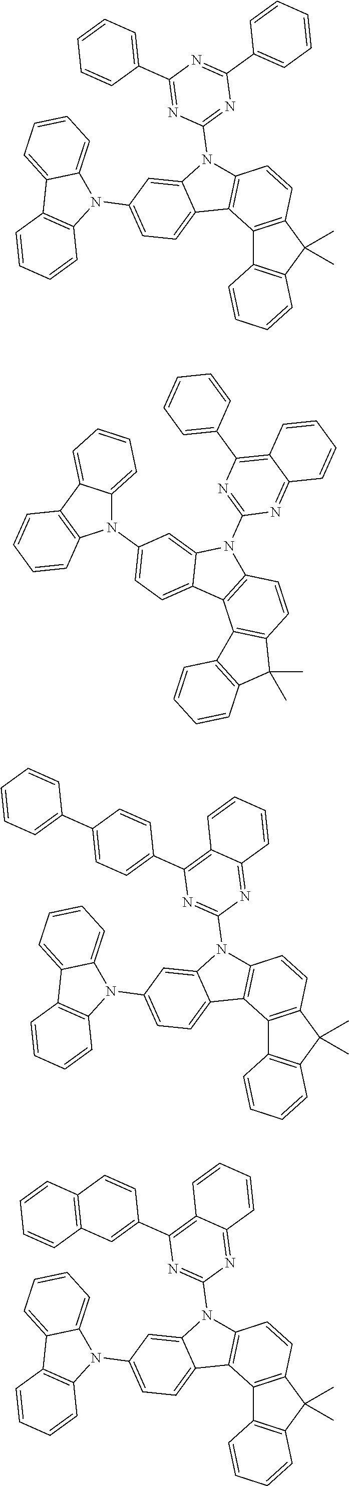 [SMILES: CC1(C)c2ccccc2-c2c1ccc1c2c2ccc(-n3c4ccccc4c4ccccc43)cc2n1-c1nc(-c2ccccc2)nc(-c2ccccc2)n1 ; CC1(C)c2ccccc2-c2c1ccc1c2c2ccc(-n3c4ccccc4c4ccccc43)cc2n1-c1nc(-c2ccccc2)c2ccccc2n1 ; CC1(C)c2ccccc2-c2c1ccc1c2c2ccc(-n3c4ccccc4c4ccccc43)cc2n1-c1nc(-c2ccc(-c3ccccc3)cc2)c2ccccc2n1 ; CC1(C)c2ccccc2-c2c1ccc1c2c2ccc(-n3c4ccccc4c4ccccc43)cc2n1-c1nc(-c2ccc3ccccc3c2)c2ccccc2n1]

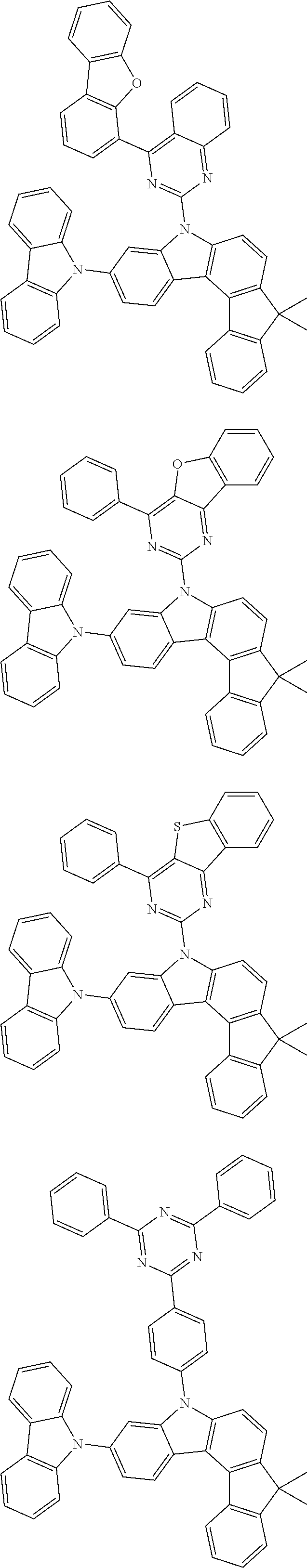

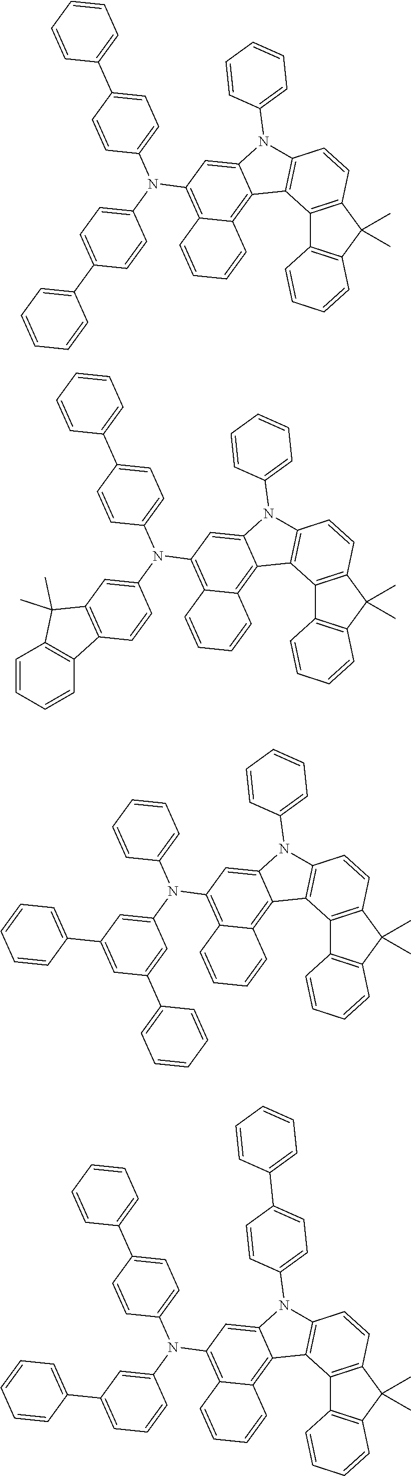

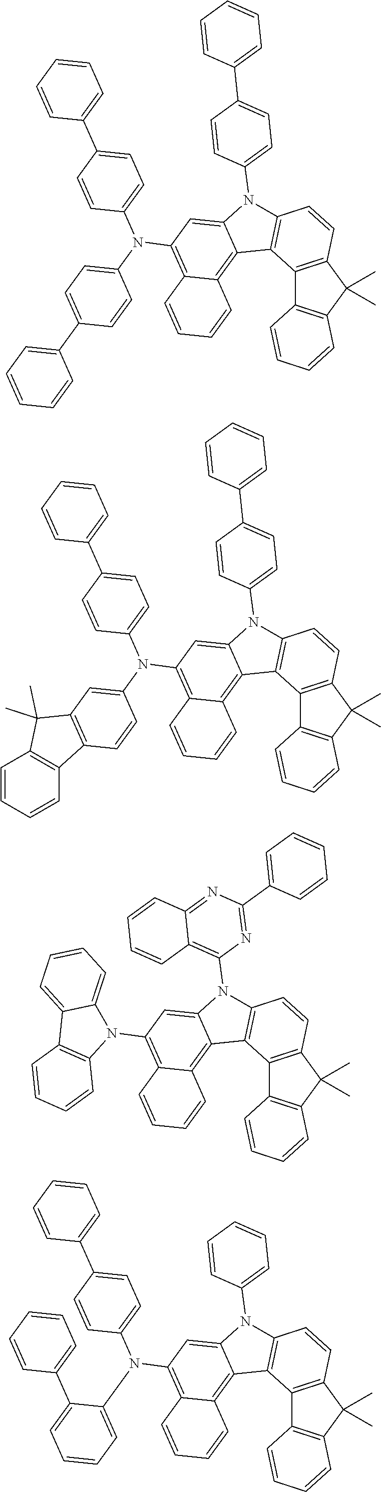

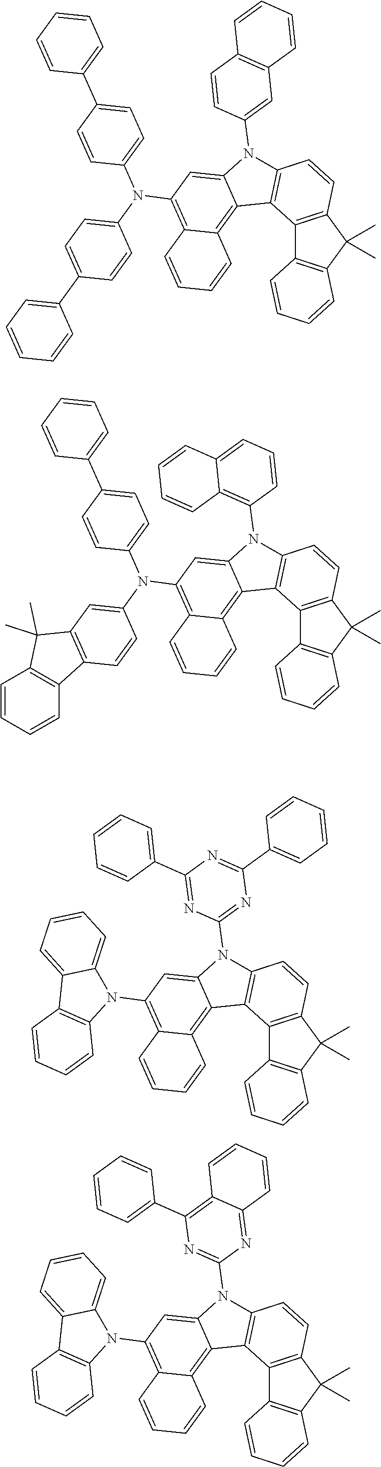

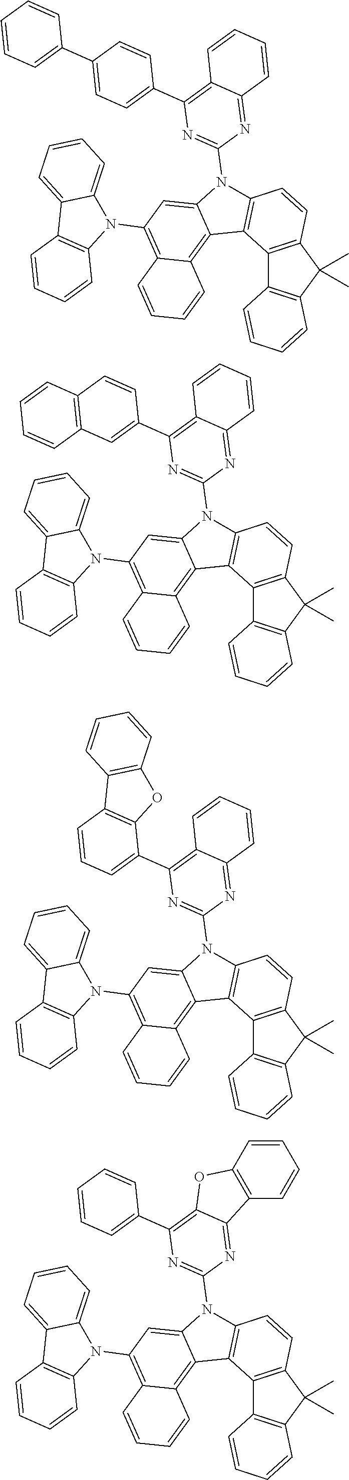

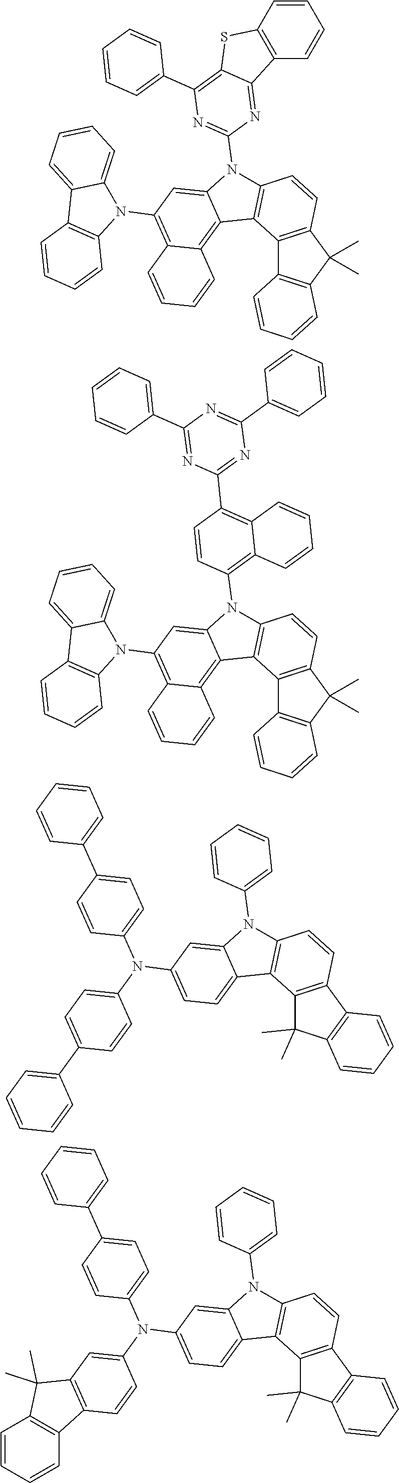

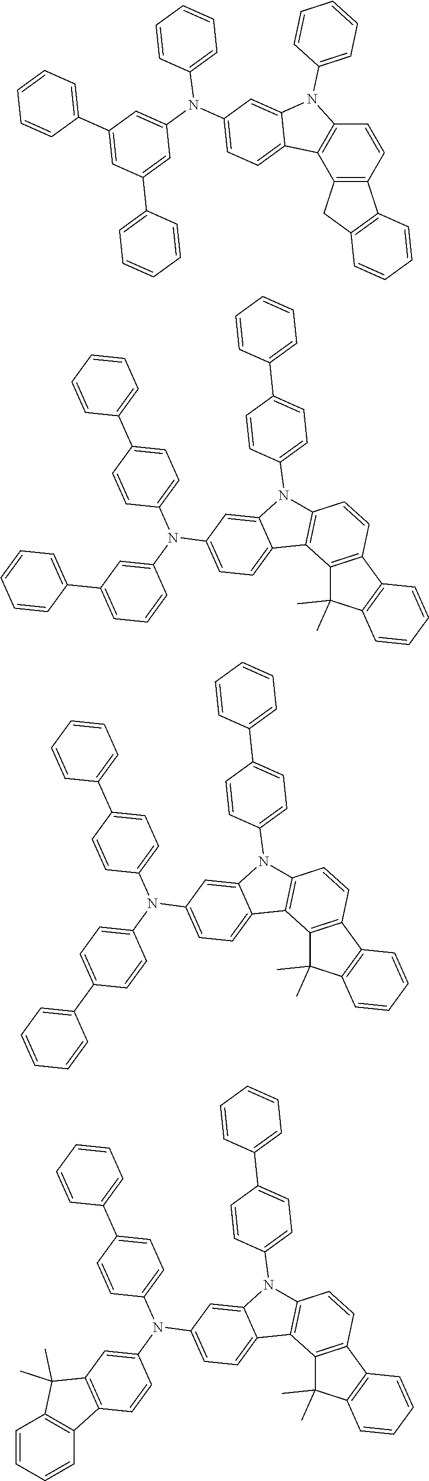 [SMILES: c1ccc(-c2cc(-c3ccccc3)cc(N(c3ccccc3)c3ccc4c5c6c(ccc5n(-c5ccccc5)c4c3)-c3ccccc3C6)c2)cc1 ; CC1(C)c2ccccc2-c2ccc3c(c21)c1ccc(N(c2ccc(-c4ccccc4)cc2)c2cccc(-c4ccccc4)c2)cc1n3-c1ccc(-c2ccccc2)cc1 ; CC1(C)c2ccccc2-c2ccc3c(c21)c1ccc(N(c2ccc(-c4ccccc4)cc2)c2ccc(-c4ccccc4)cc2)cc1n3-c1ccc(-c2ccccc2)cc1 ; CC1(C)c2ccccc2-c2ccc(N(c3ccc(-c4ccccc4)cc3)c3ccc4c5c6c(ccc5n(-c5ccc(-c7ccccc7)cc5)c4c3)-c3ccccc3C6(C)C)cc21]

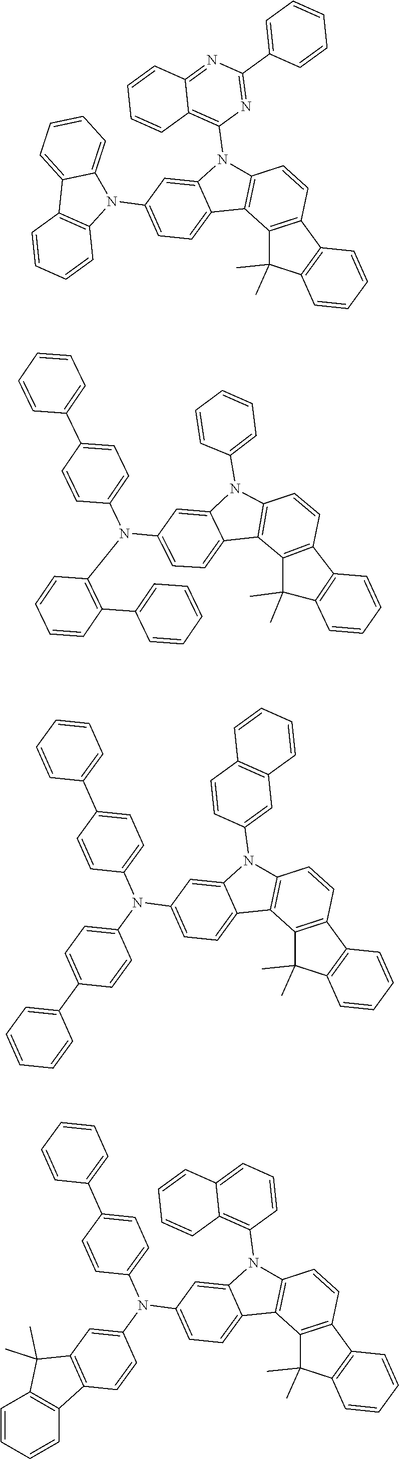 [SMILES: CC1(C)c2ccccc2-c2ccc3c(c21)c1ccc(-n2c4ccccc4c4ccccc42)cc1n3-c1nc(-c2ccccc2)nc2ccccc12 ; CC1(C)c2ccccc2-c2ccc3c(c21)c1ccc(N(c2ccc(-c4ccccc4)cc2)c2ccccc2-c2ccccc2)cc1n3-c1ccccc1 ; CC1(C)c2ccccc2-c2ccc3c(c21)c1ccc(N(c2ccc(-c4ccccc4)cc2)c2ccc(-c4ccccc4)cc2)cc1n3-c1ccc2ccccc2c1 ; CC1(C)c2ccccc2-c2ccc(N(c3ccc(-c4ccccc4)cc3)c3ccc4c5c6c(ccc5n(-c5cccc7ccccc57)c4c3)-c3ccccc3C6(C)C)cc21]

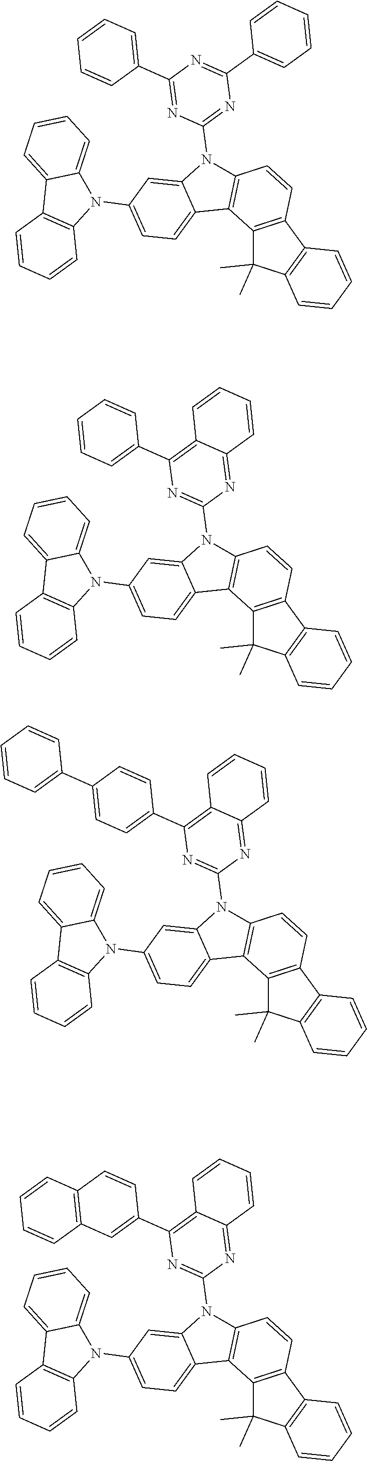 [SMILES: CC1(C)c2ccccc2-c2ccc3c(c21)c1ccc(-n2c4ccccc4c4ccccc42)cc1n3-c1nc(-c2ccccc2)nc(-c2ccccc2)n1 ; CC1(C)c2ccccc2-c2ccc3c(c21)c1ccc(-n2c4ccccc4c4ccccc42)cc1n3-c1nc(-c2ccccc2)c2ccccc2n1 ; CC1(C)c2ccccc2-c2ccc3c(c21)c1ccc(-n2c4ccccc4c4ccccc42)cc1n3-c1nc(-c2ccc(-c3ccccc3)cc2)c2ccccc2n1 ; CC1(C)c2ccccc2-c2ccc3c(c21)c1ccc(-n2c4ccccc4c4ccccc42)cc1n3-c1nc(-c2ccc3ccccc3c2)c2ccccc2n1]

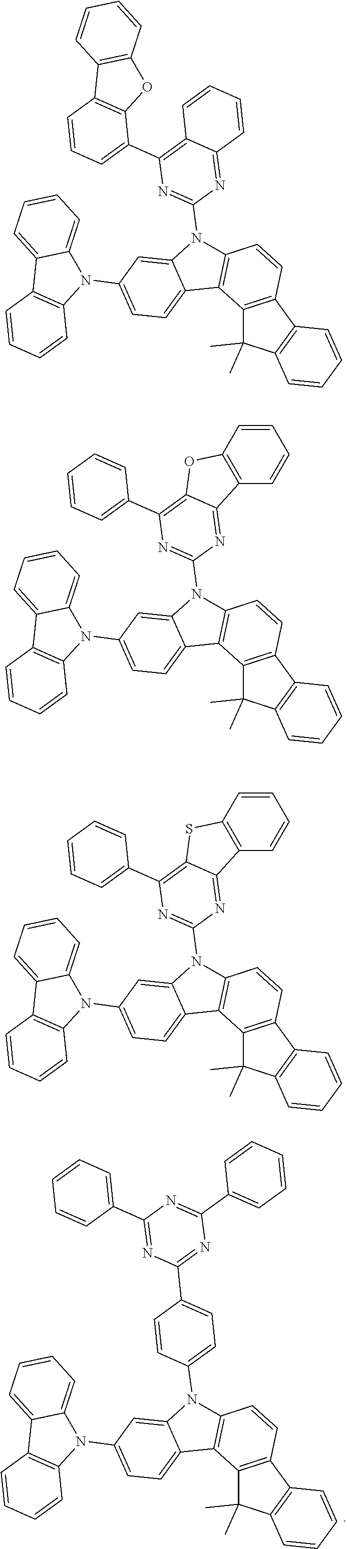

6. An organic light emitting device, comprising:
a first electrode;
a second electrode provided opposite to the first electrode; and
one or more organic material layers provided between the first electrode and the second electrode, wherein one or more one layers of the organic material layers comprises the compound of claim 1.

7. An organic light emitting device, comprising:
a first electrode;
a second electrode provided opposite to the first electrode; and
one or more organic material layers provided between the first electrode and the second electrode, wherein one or more one layers of the organic material layers comprises the compound of claim 2.

8. An organic light emitting device, comprising:
a first electrode;
a second electrode provided opposite to the first electrode; and
one or more organic material layers provided between the first electrode and the second electrode, wherein one or more one layers of the organic material layers comprises the compound of claim 3.

9. An organic light emitting device, comprising:
a first electrode;
a second electrode provided opposite to the first electrode; and
one or more organic material layers provided between the first electrode and the second electrode, wherein one or more one layers of the organic material layers comprises the compound of claim 4.

10. An organic light emitting device, comprising:
a first electrode;
a second electrode provided opposite to the first electrode; and
one or more organic material layers provided between the first electrode and the second electrode, wherein one or more one layers of the organic material layers comprises the compound of claim 5.
